(19) United States
(12) Patent Application Publication
(10) Pub. No.: US 2011/0064650 A1
(43) Pub. Date: Mar. 17, 2011

(54) MICROORGANISMS FOR PREVENTING AND TREATING NEOPLASMS ACCOMPANYING CELLULAR THERAPY

(76) Inventor: Aladar A. Szalay, Highland, CA (US)

(21) Appl. No.: 12/736,826

(22) PCT Filed: May 15, 2009
(86) PCT No.: PCT/US2009/003061

§ 371 Date: Nov. 10, 2010

Related U.S. Application Data
(60) Provisional application No. 61/054,025, filed on May 16, 2008.

(51) Int. Cl. A61K 51/00; A61K 35/76; A61K 49/00; A61B 5/055; A61P 35/00

(52) U.S. Cl. 424/1.11; 424/93.6; 424/9.1; 424/9.6; 424/9.3

(57) ABSTRACT

Provided are methods for using cellular compositions in combination with oncolytic viruses. The methods include administering oncolytic viruses for the inhibition and treatment of tumors caused by administration of cellular therapies, such as stem cell therapies. The methods also include contacting cellular compositions with oncolytic viruses for the removal of neoplastic cells prior to administration of the cellular composition for therapy. Diagnostic methods for monitoring treatment also are provided.

### RELATED APPLICATIONS

Benefit of priority is claimed to U.S. Provisional Application Ser. No. 61/054,025, to Aladar A. Szalay, filed on May 16, 2008, entitled “MICROORGANISMS FOR STEM CELL TREATMENTS.” Where permitted, the subject matter of this application is incorporated by reference in its entirety.

This application is related to U.S. application Ser. No. 12/218,953, filed on Jul. 18, 2008, entitled “USE OF MODIFIED VACCINIA VIRUS STRAINS IN COMBINATION WITH A CHEMOTHERAPEUTIC AGENT FOR USE IN THERAPEUTIC METHODS,” to International Application No. PCT/US2008/008832, filed on Jul. 18, 2008, entitled “USE OF MODIFIED VACCINIA VIRUS STRAINS IN COMBINATION WITH A CHEMOTHERAPEUTIC AGENT FOR USE IN THERAPEUTIC METHODS.”

This application also is related to U.S. application Ser. No. 11/975,088, filed on Oct. 16, 2007, entitled “METHODS FOR ATTENUATING VIRUS STRAINS FOR DIAGNOSTIC AND THERAPEUTIC USES,” to U.S. application Ser. No. 11/975,090, filed on Oct. 16, 2007, entitled “MODIFIED VACCINIA VIRUS STRAINS FOR USE IN DIAGNOSTIC AND THERAPEUTIC METHODS,” to U.S. application Ser. No. 12/080,766, filed on Apr. 4, 2008, entitled “MODIFIED VACCINIA VIRUS STRAINS FOR USE IN DIAGNOSTIC AND THERAPEUTIC METHODS,” and to International Application No. PCT/US2007/022172, filed on Oct. 16, 2007, entitled “MODIFIED VACCINIA VIRUS STRAINS FOR USE IN DIAGNOSTIC AND THERAPEUTIC METHODS.”

This application also is related to U.S. application Ser. No. 12/157,960, filed on Jun. 13, 2008, entitled “MICROORGANISMS FOR IMAGING AND/OR TREATMENT OF TUMORS” and to International Application No. PCT/US2008/007377, filed on Jun. 13, 2008, entitled “MICROORGANISMS FOR IMAGING AND/OR TREATMENT OF TUMORS.”

This application is related to U.S. application Ser. No. 10/872,156, filed on Jun. 18, 2004, entitled “MICROORGANISMS FOR THERAPY,” which claims the benefit of priority under 35 U.S.C. §119(a) to each of EP Application No. 03 013 826.7, filed 18 Jun. 2003, entitled “Recombinant vaccinia viruses useful as tumor-specific delivery vehicle for cancer gene therapy and vaccination,” EP Application No. 03 018 478.2, filed 14 Aug. 2003, entitled “Method for the production of a polypeptide, RNA or other compound in tumor tissue,” and EP Application No. 03 024 283.8, filed 22 Oct. 2003, entitled “Use of a Microorganism or Cell to Induce Autoimmunization of an Organism Against a Tumor.” This application also is related to International Application No. PCT/US04/19866, filed on Jun. 18, 2004, entitled “MICROORGANISMS FOR THERAPY.”

This application also is related to U.S. application Ser. No. 10/866,606, filed Jun. 10, 2004, entitled “LIGHT EMITTING MICROORGANISMS AND CELLS FOR DIAGNOSIS AND THERAPY OF TUMORS,” which is a continuation of U.S. application Ser. No. 10/189,918, filed Jul. 3, 2002, entitled “LIGHT EMITTING MICROORGANISMS AND CELLS FOR DIAGNOSIS AND THERAPY OF TUMORS.” This application also is related to International PCT Application PCT/IB02/04767, filed Jul. 31, 2002, entitled “MICROORGANISMS AND CELLS FOR DIAGNOSIS AND THERAPY OF TUMORS,” EP Application No. 01 118 417.3, filed Jul. 31, 2001, entitled “Light-emitting microorganisms and cells for tumor diagnosis/therapy,” EP Application No. 01 125 911.6, filed Oct. 30, 2001, entitled “LIGHT EMITTING MICROORGANISMS AND CELLS FOR DIAGNOSIS AND THERAPY OF TUMORS” and EP Application No. 02 0794 632.6, filed Jan. 28, 2004, entitled “Microorganisms and Cells for Diagnosis and Therapy of Tumors.”

Where permitted, the subject matter of each of the above applications is incorporated by reference in its entirety.

### FIELD OF INVENTION

Methods for using cellular compositions in combination with oncolytic viruses are provided. Methods for using cellular compositions in combination with oncolytic viruses for the inhibition and treatment of tumors are provided. Diagnostic and therapeutic methods also are provided.

### BACKGROUND

Cell compositions that are administered to a subject in cell therapy protocols have the potential to result in the formation of a tumor, insofar as the cell composition can contain neoplastic cells or neoplastic progenitor cells. For example, cell therapies, such as administration of stem cells, have been associated with formation of malignant cancers in subjects receiving stem cell therapies. While the potential of cellular therapy in the treatment of diseases, disorders and injury is significant, the formation of tumors, such as teratomas, as a result of such treatment is an unacceptable outcome. Accordingly, there exists a need for methods of safely administering cellular compositions that reduces the risk of tumor formation in subjects receiving cellular therapy.

### SUMMARY

Provided herein are methods for inhibiting the development of a tumor or inhibiting tumor cells in a subject receiving a cell therapy, where the method involves administering a cellular composition to a subject in combination with an oncolytic virus to the subject. In such methods, the oncolytic virus is selected from those that infect neoplastic cells of the cellular composition. The cellular composition can contain cells, such as, but not limited to, stem cells, bone marrow cells, immune cells, or cells comprising a gene therapy vector. In some examples, the tumor that is inhibited is a teratoma.

In examples where the cells for the cellular therapy are immune cells, the cells can be T lymphocytes, antigen presenting cells or natural killer cells. T lymphocytes include, for example, tumor-infiltrating lymphocytes (TIL) or cytotoxic lymphocytes (CTL). The TIL can be harvested from a subject and treated with an immunostimulatory agent, such as interleukin-2.

In some examples, the oncolytic virus and the cellular composition are administered simultaneously, sequentially or intermittently. In some examples, the cellular composition is administered in a single administration or multiple administrations. In some examples, the cellular composition is administered locally or systemically. For example, the cellular composition can be administered to the subject intravenously, intraarterially, intratumorally, endoscopically, intralesionally, intramuscularly, intradermally, intraperitoneally, intravesicularly, intraarticularly, intrapleurally, percutaneously, subcutaneously, orally, parenterally, intranasally, intratracheally, by inhalation, intracranially, intraprostaticaly, intravitreally, ocularly, vaginally, intracoronary, intramyocardially, transendocardially, trans-epicardially, intraspinally, intra-striatumly, transdermally, rectally or sub-epidermally. In particular examples, the cellular composition is administered by injection at a site of tissue or cell damage.

In some examples, the oncolytic virus is administered in a single administration or multiple administrations. In some examples, the oncolytic virus is administered locally or systemically. For example, the virus can be administered intravenously, intraarterially, intratumorally, endoscopically, intralesionally, intramuscularly, intradermally, intraperitoneally, intravesicularly, intraarticularly, intrapleurally, percutaneously, subcutaneously, orally, parenterally, intranasally, intratracheally, by inhalation, intracranially, intraprostaticaly, intravitreally, topically, ocularly, vaginally, or rectally.

Provided herein are methods for removing neoplastic cells or neoplastic progenitors from a cellular composition, comprising contacting a cellular composition with a virus that infects and kills neoplastic ells, wherein the virus is an LIVP virus. Also, provided herein are methods for inhibiting the formation of a tumor in a subject receiving a cellular therapy, where the method involves contacting a cellular composition with a virus that infects and kills neoplastic cells and subsequently administering the treated cellular composition to a subject for therapy. In some examples, the cellular composition can be contacted with the virus, for example, for 1, 2, 3, 4, 5, 6, 7, 8, 9, 10, 11, 12, 18, 24, 30, 36, 42, 48, or 72 hours. In some examples, the cellular composition is contacted with the virus at multiplicity of infection of about or 0.001, 0.01, 0.1, 1, 10, or 100. In exemplary methods, the cellular composition can contain cells, such as, but not limited to, stem cells, bone marrow cells, immune cells, or cells comprising a gene therapy vector. In some examples where the cells for the cellular therapy are immune cells, the cells can be T lymphocytes, antigen presenting cells or natural killer cells. T lymphocytes include, for example, tumor-infiltrating lymphocytes (TIL) or cytotoxic lymphocytes (CTL). The TIL can be harvested from a subject and treated with an immunostimulatory agent, such as interleukin-2.

In some examples of the methods provided, the cellular therapy is administered for treatment of a disease or disorder, such as, but not limited to, cardiovascular disease, cancer, diabetes, spinal cord injury, neurodegenerative disease, Alzheimer's disease, Parkinson's disease, multiple sclerosis, Amyotrophic lateral sclerosis, Duchenne Muscular Dystrophy, muscle damage or dystrophy, stroke, burns, lung disease, retinal disease, kidney disease, osteoarthritis, and rheumatoid arthritis.

In some examples, the cellular composition for use in the methods provided is a stem cell composition. For example, the stem cell composition can contain embryonic stem cells, germinal stem cells, or adult stem cells. In some examples, the stem cells are mammalian embryonic stem cells. In some examples, the embryonic stem cells are human embryonic stem cells. In other examples, the embryonic stem cells are non-human stem cells, such as, for example, mouse, rat or non-human primate embryonic stem cells. In some examples, the embryonic stem cell composition for use in the methods provided contain ACT-14, AS034, AS034.1, AS034.2, AS038, AS079, AS094, BG01, BG02, BG03, BG04, CH01, CH02, CLS1, CLS2, CLS3, CLS4, ES01, ES02, ES03, ES04, ES05, ES06, ESM01, ESM02, ESM03, FC018, FES 21, FES 22, FES 29, FES 30, GE01, GE07, GE09, GE13, GE14, GE91, GE92, hES-NCL1, HS181, HS207, HUES1, HUES10, HUES11, HUES12, HUES13, HUES14, HUES15, HUES16, HUES17, HUES2, HUES3, HUES4, HUES5, HUES6, HUES7, HUES8, HUES9, KhES-1, KhES-2, KhES-3, MB01, MB02, MB03, Miz-hES1, Miz-hES10, Miz-hES11, Miz-hES12, Miz-hES13, Miz-hES14, Miz-hES15, Miz-hES2, Miz-hES3, Miz-hES4, Miz-hES5, Miz-hES6, Miz-hES7, Miz-hES8, NC01, NC02, NC03, ReliCellhES1, RH1, RH3, RH4, RH5, RH6, RH7, RL05, RL07, RL10, RL13, RL15, RL20, RL21, Royan H1, SA001, SA002, SA002.5, SA046, SA085, SA111, SA121, SA142, SA167, SA181, SA191, SA196, SA202, SA203, SA211, SA218, SA240, SA279, SA348, SA352, SA399, SA611, SI-100, SI-101, SI-102, SI-103, SI-104, SI-105, SI-106, SI-107, SI-108, SI-109, SI-110, SI-111, SI-114, SI-115, SI-122, SI-123, SI-124, SI-125, SI-126, SI-128, SI-130, SI-131, SI-132, SI-133, SI-134, SI-135, SI-137, SI-138, SI-139, SI-140, SI-141, SI-144, SI-145, SI-146, SI-148, SI-149, SI-15, SI-150, SI-151, SI-153, SI-154, SI-155, SI-156, SI-157, SI-158, SI-159, SI-160, SI-161, SI-162, SI-163, SI-164, SI-165, SI-167, SI-168, SI-169, SI-170, SI-171, SI-172, SI-174, SI-175, SI-176, SI-177, SI-178, SI-179, SI-18, SI-180, SI-182, SI-183, SI-184, SI-185, SI-186, SI-187, SI-188, SI-189, SI-191, SI-192, SI-193, SI-194, SI-195, SI-196, SI-197, SI-198, SI-199, SI-200, SI-201, SI-202, SI-203, SI-204, SI-205, SI-206, SI-208, SI-209, SI-21, SI-210, SI-211, SI-213, SI-214, SI-215, SI-216, SI-217, SI-221, SI-24, SI-27, SI-28, SI-31, SI-33, SI-53, SI-60, SI-62, SI-63, SI-79, SI-80, SI-81, SI-93, SI-94, SI-95, SI-96, SI-97, SI-98, SI-99, SNUhES1, SNUhES2, SNUhES3, TE-03, TE-04, TE-06, TE-07, TE-32, TE-33, TE-62, TE-72, UC01, UC06, VAL-1, VAL-2, VAL-3, VAL-4, WA01, WA07, WA09, WA13 or WA14 cells.

In some examples where the stem cell composition is an adult stem cell composition, the composition can contain hematopoietic stem cells, mesenchymal stem cells, or multipotent adult progenitor cells. In some examples, the hematopoietic stem cells are harvested from bone marrow or blood.

In some examples, the stem cells in the stem cell compositions have been partially differentiated in vitro prior to administration to the subject or prior to contact with the virus. For example, the stem cells can be differentiated in vitro for 1, 2, 3, 4, 5, 6, 7, 8, 9, 10, 11, 12, 13, 14, 15, 16, 17, 18, 19, 20, 25 or 30 days.

In any of the exemplary methods provided, the virus can be a vaccinia virus. For example, the virus can be a Lister strain virus, such as, for example, LIVP. In some examples, virus is selected from among GLV-1h68, GLV-1i69, GLV-1h70, GLV-1h71, GLV-1h72, GLV-1h73, GLV-1h74, GLV-1h81, GLV-1h82, GLV-1h83, GLV-1h84, GLV-1h85, GLV-1h86, GLV-1j87, GLV-1j88, GLV-1j89, GLV-1h90, GLV-1h91, GLV-1h92, GLV-1h96, GLV-1h97, GLV-1h98, GLV-1h100, GLV-1h101, GLV-1h139, GLV-1h104, GLV-1h105, GLV-1h106, GLV-1h107, GLV-1h108, GLV-1h109, GLV-1h146, GLV-1h150, GLV-1h151, GLV-1h152 and GLV-1h153.

In some examples of the methods where the virus is administered directly to a subject, the amount of virus administered is 1×10⁵ or about 1×10⁵ plaque forming units (PFU), 5×10⁵ or about 5×10⁵ PFU, at least 1×10⁶ or about 1×10⁶ PFU, 5×10⁶ or about 5×10⁶ PFU, 1×10⁷ or about 1×10⁷ PFU, 5×10⁷ or about 5×10⁷ PFU, 1×10⁸ or about 1×10⁸ PFU, 5×10⁸ or about 5×10⁸ PFU, 1×10⁹ or about 1×10⁹ PFU, 5×10⁹ or about 5×10⁹ PFU, 1×10¹⁰ or about 1×10¹⁰ PFU or 5×10¹⁰ or about 5×10¹⁰ PFU.

Provided herein are methods for inhibiting the development of a tumor or inhibiting tumor cells in a subject receiving a cell therapy, where the method involves administering a cellular composition to a subject in combination with an oncolytic virus to the subject and detecting the virus in the subject. In some examples, the virus is detected by fluorescence imaging, magnetic resonance imaging (MRI), single-photon emission computed tomography (SPECT), positron emission tomography (PET), scintigraphy, gamma camera, a β+ detector, a γ detector or a combination of such methods. In some examples, the virus encodes a detectable protein or a protein that induces a detectable signal. For example, the detectable protein can be a luciferase or a fluorescent protein.

The virus for use in any of the methods provided can encode a therapeutic gene product. In some examples, the therapeutic gene product is an anti-cancer agent or anti-angiogenic agent. For example, the therapeutic gene product can be selected from among a cytokine, a chemokine, an immunomodulatory molecule, an antigen, an antibody or fragment thereof, antisense RNA, prodrug converting enzyme, siRNA, angiogenesis inhibitor, a toxin, an antitumor oligopeptide, a mitosis inhibitor protein, an antimitotic oligopeptide, an anti-cancer polypeptide antibiotic, a transporter protein, and tissue factor.

In methods provided herein for administering a cellular composition to a subject, the method can also include administering an anticancer agent. Exemplary anticancer agents included, but are not limited to, a cytokine, a chemokine, a growth factor, a photosensitizing agent, a toxin, an anti-cancer antibiotic, a chemotherapeutic compound, a radionuclide, an angiogenesis inhibitor, a signaling modulator, an anti-metabolite, an anti-cancer vaccine, an anti-cancer oligopeptide, a mitosis inhibitor protein, an antimitotic oligopeptide, an anti-cancer antibody, an anti-cancer antibiotic, an immunotherapeutic agent, hyperthermia or hyperthermia therapy, a bacterium, radiation therapy and a combination of such agents. In some examples, the anticancer agent is cisplatin, carboplatin, gemcitabine, irinotecan, an anti-EGFR antibody, or an anti-VEGF antibody. In some examples, the anticancer agent is administered simultaneously, sequentially, or intermittently with the virus.

In methods provided herein for administering an oncolytic virus to a subject, the method can also include administering an anti-viral agent to attenuate replication of or eliminate the virus from the subject during or following therapy. Exemplary antiviral agents, include, but are not limited to cidofovir, alkoxyalkyl esters of cidofovir, Gleevec, gancyclovir, acyclovir and ST-26.

Provided herein are uses of a virus for formulation of a medicament for preventing tumor formation in a subject receiving a cellular therapy. Also, provided herein are viruses for use in preventing tumor formation in a subject receiving a cellular therapy. In some examples, the therapy is a stem cell therapy, including, but not limited to, treatment of cardiovascular disease, cancer, diabetes, spinal cord injury, neurodegenerative disease, Alzheimer's disease, Parkinson's disease, multiple sclerosis, Amyotrophic lateral sclerosis, Duchenne Muscular Dystrophy, muscle damage or dystrophy, stroke, burns, lung disease, retinal disease, kidney disease, osteoarthritis or rheumatoid arthritis. In some examples, the virus is a vaccinia virus. For example, the virus can be a Lister strain virus, such as, for example, LIVP. In some examples, virus is selected from among GLV-1h68, GLV-1i69, GLV-1h70, GLV-1h71, GLV-1h72, GLV-1h73, GLV-1h74, GLV-1h81, GLV-1h82, GLV-1h83, GLV-1h84, GLV-1h85, GLV-1h86, GLV-1j87, GLV-1j88, GLV-1j89, GLV-1h90, GLV-1h91, GLV-1h92, GLV-1h96, GLV-1h97, GLV-1h98, GLV-1h100, GLV-1h101, GLV-1h139, GLV-1h104, GLV-1h105, GLV-1h106, GLV-1h107, GLV-1h108, GLV-1h109, GLV-1h146, GLV-1h150, GLV-1h151, GLV-1h152 and GLV-1h153. In some examples, the virus encodes a detectable protein or a protein that induces a detectable signal, such as, for example, a luciferase or a fluorescent protein. In some examples, the virus encodes a therapeutic gene product, such as, for example, an anti-cancer agent or anti-angiogenic agent. Exemplary anti-cancer agents include, but are not limited to, a cytokine, a chemokine, an immunomodulatory molecule, an antigen, an antibody or fragment thereof, antisense RNA, prodrug converting enzyme, siRNA, angiogenesis inhibitor, a toxin, an antitumor oligopeptide, a mitosis inhibitor protein, an antimitotic oligopeptide, an anti-cancer polypeptide antibiotic, a transporter protein, and tissue factor.

### DETAILED DESCRIPTION

A. Definitions
B. Overview
C. Cellular therapy

1. Bone marrow transplant
2. T cell therapy
3. Stem cell therapy

a. Stem cells

Embryonic stem cells

b. Conditions amenable to stem cell therapy
c. Stem cell-associated tumors

D. Therapeutic and Diagnostic Methods for the Treatment and

Prevention of Cell therapy-associated Tumors

1. Viruses For Use in the Methods Provided

a. Exemplary viruses

i. Poxviruses

(1) Vaccinia viruses
(2) Modification of Vaccinia Viruses
(3) Exemplary Modified Vaccinia Viruses

b. Other cytoplasmic viruses
c. Adenovirus, Herpes, Retroviruses

2. Exemplary cullular compositions
3. Methods of Treatment

a. Administration of Virus for Prevention of Tumor
Formation

i. Direct Administration of Virus

(1) Mode of administration

ii. Pre-treatment of Stem Cells for Administration
iii. Administration of virus for treatment of a stem

cell-derived tumor
b. Virus Dosages
c. Number of administrations
d. Steps prior to administration of the virus

4. Co-administrations

a. Administering a plurality of viruses
b. Therapeutic Compounds
c. Immunotherapies and biological therapies

5. Monitoring

a. Monitoring viral gene expression
b. Monitoring tumor size
c. Monitoring antibody titer
d. Monitoring general health diagnostics
e. Monitoring coordinated with treatment

E. Therapeutic and Diagnostic Methods for the Treatment and

Prevention of Tumors Associated with Other Cell Therapies

F. Pharmaceutical Compositions, Combinations and Kits

1. Pharmaceutical Compositions
2. Combinations
3. Kits

G. Examples

### A. DEFINITIONS

Unless defined otherwise, all technical and scientific terms used herein have the same meaning as is commonly understood by one of skill in the art to which the invention(s) belong. All patents, patent applications, published applications and publications, websites and other published materials referred to throughout the entire disclosure herein, unless noted otherwise, are incorporated by reference in their entirety. In the event that there are pluralities of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information.

As used herein, “virus” refers to any of a large group of entities referred to as viruses, which typically contain a protein coat surrounding an RNA or DNA core of genetic material, and are capable of growth and multiplication only in living cells. Viruses for use in the methods provided herein include, but are not limited, to a poxvirus, including a vaccinia virus (e.g. a Lister strain vaccinia virus, such as LIVP). Other exemplary viruses include, but are not limited to, adenovirus, adeno-associated virus, herpes simplex virus, Newcastle disease virus, vesicular stomatitis virus, mumps virus, influenza virus, measles virus, reovirus, human immunodeficiency virus (HIV), hanta virus, myxoma virus, cytomegalovirus (CMV), lentivirus, Sindbis virus, and any plant or insect virus.

As used herein, an “oncolytic virus” is a replication competent virus that preferentially replicates in, and kills, neoplastic or cancer cells. The virus can be a naturally-occurring virus or an engineered virus. In some examples provide herein, the oncolytic virus is a modified vaccinia virus.

As used herein, “neoplastic cells” refer to cells which exhibit relatively autonomous growth, so that they exhibit an aberrant growth phenotype characterized by a significant loss of control of cell proliferation. Neoplastic cells comprise cells which may be actively replicating or in a temporary non-replicative resting state (G 1 or G 0); similarly, neoplastic cells may comprise cells which have a well-differentiated phenotype, a poorly-differentiated phenotype, or a mixture of both type of cells. Thus, not all neoplastic cells are necessarily replicating cells at a given timepoint. Neoplastic cells encompasses such cells in benign neoplasms and cells in malignant neoplasms. Malignant neoplastic cells are frequently referred to as cancer, typically termed carcinoma if originating from cells of endodermal or ectodermal histological origin, or sarcoma if originating from cell types derived from mesoderm.

As used herein, “neoplastic progenitor cells” refers to cells of a cellular composition that possess the ability to become neoplastic.

As used herein, the term “neoplasm” or “neoplasia” refers to abnormal new cell growth, and thus means the same as tumor, which can be benign or malignant. Unlike hyperplasia, neoplastic proliferation persists even in the absence of the original stimulus.

As used herein, a “stem cell-derived tumor” is any tumor that originates from cells or progeny cells of an exogenous stem cell composition that is administered to a subject. The stem cell-derived tumor can be benign or malignant.

As used herein, a “cellular composition” or “cell composition” is any composition that contains cells. The cell composition may be a mixed cellular composition with two or more types of cells. The cell compositions described herein typically contain normal and neoplastic cells. As described herein, oncolytic viruses can be administered to eliminate neoplastic cells from a cellular composition in vivo or in vitro.

As used herein, a “stem cell composition” is any cellular composition containing a population of cells, some of which are stem cells. The composition also can contain cells that are not stem cells or non-cellular matter, such as for example, hormones, peptides or other extracellular material.

As used herein, a “stem cell” is any totipotent, pluripotent or multipotent cell that has the ability to differentiate into multiple different types of cells (e.g., terminally differentiated cells). For example, stem cells include those that can differentiate into any of the three main germ layers: endoderm, ectoderm, and mesoderm. Stem cells include any type of stem cell, such as embryonic stem cells, post natal stem cells (e.g. from the umbilical cord and placenta) adult stem cells, fetal stem cells or artificially engineered stem cells.

As used herein, “embryonic stem cells” are stem cells obtained from an embryo that is typically six weeks old or less. Totipotent human embryonic stem cells (hESC) generally can be obtained from embryos that are 5 to 7 days old. Pluripotent human primordial germ cells (hEG) typically can be obtained from embryos that are six weeks old or less.

As use herein, “fetal stem cells” refer to any stem cell that is obtained prenatally from a fetus that is typically greater that 6 weeks old. Both pluripotent and multipotent human stem cells (hSC) are typically.

As used herein, “adult stem cells” refers to any stem cell that is obtained from a post-natal subject. Typically, the subject is a full grown adult. Exemplary adult stem include, but are not limited to cells harvested from organs such as fat, muscle or bone marrow.

As used herein, an “exogenous stem cell composition” is any stem cell composition that is administered to a subject for stem cell therapy. The exogenous stem cell composition can contain stem cells harvested from the subject or can contain stem cell obtained from other sources, such as for example, embryonic stem cells or adult stem cells from another donor.

As used herein, the term “exogenous” is used interchangeably with the term “heterologous” refer to a substance coming from some source other than its native source. For example, the terms “exogenous protein,” or “exogenous cell” refer to a protein or cell from a non-native source or location, and that have been artificially supplied to a biological system. In contrast, the terms “endogenous protein,” or “endogenous cell” refer to a protein or cell that are native to the biological system, species or individual.

As used herein, “inhibition of the formation of a tumor” or “inhibition of the development of a tumor” includes prevention of tumor development or formation, or lessoning in the risk of development of a tumor in a subject as the result of administering cellular therapy or, in the event a tumor has formed as a result of the therapy, it refers to treatment of the tumor, including eradication thereof.

As used herein, “stem cell therapy” refers to any use of a stem cell composition to treat a disease or disorder.

As used herein, the phrase “contacting a stem cell composition with a virus” refers to the addition of a virus to a stem cell culture in order to infect cells of the stem cell culture with the virus.

As used herein, a “pretreated stem cell composition” is a stem cell composition that has been infected with an oncolytic virus for a predetermined period of time.

As used herein, neoplastic disease refers to any disorder involving cancer, including tumor development, growth, metastasis and progression.

As used herein, the term “viral vector” is used according to its art-recognized meaning. It refers to a nucleic acid vector construct that includes at least one element of viral origin and can be packaged into a viral vector particle. The viral vector particles can be used for the purpose of transferring DNA, RNA or other nucleic acids into cells either in vitro or in vivo. Viral vectors include, but are not limited to, retroviral vectors, vaccinia vectors, lentiviral vectors, herpes virus vectors (e.g., HSV), baculoviral vectors, cytomegalovirus (CMV) vectors, papillomavirus vectors, simian virus (SV40) vectors, semliki forest virus vectors, phage vectors, adenoviral vectors, and adeno-associated viral (AAV) vectors.

As used herein, the term “modified” with reference to a gene refers to a deleted gene, a gene encoding a gene product having one or more truncations, mutations, insertions or deletions, or a gene that is inserted (into the chromosome or on a plasmid, phagemid, cosmid, and phage) encoding a gene product, typically accompanied by at least a change in function of the modified gene product or virus.

As used herein, the term “modified virus” refers to a virus that is altered with respect to a parental strain of the virus. Typically modified viruses have one or more truncations, mutations, insertions or deletions in the genome of virus. A modified virus can have one or more endogenous viral genes modified and/or one or more intergenic regions modified. Exemplary modified viruses can have one or more heterologous nucleic acid sequences inserted into the genome of the virus. Modified viruses can contain one more heterologous nucleic acid sequences in the form of a gene expression cassette for the expression of a heterologous gene. As used herein, modification of a heterologous nucleic acid molecule with respect to a virus containing a heterologous nucleic acid molecule refers to any alteration of the heterologous nucleic acid molecule including truncations, mutations, insertions, or deletions of the nucleic acid molecule. Modification of a heterologous nucleic acid molecule can also include alteration of the viral genome, which can be, for example, a deletion of all or a portion heterologous nucleic from the viral genome or insertion of an additional heterologous nucleic acid molecule into the viral genome.

As used herein, the term “therapeutic virus” refers to a virus that is administered for the treatment of a disease or disorder. A therapeutic virus is typically a modified virus. Such modifications include one or more insertions, deletions, or mutations in the genome of the virus. Therapeutic viruses typically possess modifications in one or more endogenous viral genes or one or more intergenic regions, which attenuate the toxicity of the virus, and can optionally express a heterologous therapeutic gene product and/or detectable protein. Therapeutic viruses can contain heterologous nucleic acid molecules, including one or more gene expression cassettes for the expression of the therapeutic gene product and/or detectable protein. Therapeutic viruses can be replication competent viruses (e.g., oncolytic viruses) including conditional replicating viruses, or replication-defective viruses. As used herein, the term, “therapeutic gene product” refers to any heterologous protein expressed by the therapeutic virus that ameliorates the symptoms of a disease or disorder or ameliorates the disease or disorder.

As used herein, attenuation of a virus means to a reduction or elimination of deleterious or toxic effects to a host upon administration of the virus compared to an un-attenuated virus. As used herein, a virus with low toxicity means that upon administration a virus does not accumulate in organs and tissues in the host to an extent that results in damage or harm to organs, or that impacts survival of the host to a greater extent than the disease being treated does. For the purposes herein, attenuation of toxicity is used interchangeably with attenuation of virulence and attenuation of pathogenicity.

As used herein, the term “viral load” is the amount of virus present in the blood of a patient. Viral load is also referred to as viral titer or viremia. Viral load can be measured in variety of standard ways, including immunochemistry methods or by plaque assay.

As used herein, the term “toxicity” with reference to a virus refers to the ability of the virus to cause harm to the subject to which the virus has been administered.

As used herein virulence and pathogenicity with reference to a virus refers to the ability of the virus to cause disease or harm in the subject to which the virus has been administered. Hence, for the purposes herein the terms toxicity, virulence, and pathogenicity with reference to a virus are used interchangeably.

As used herein, a disease or disorder refers to a pathological condition in an organism resulting from, for example, infection or genetic defect, and characterized by identifiable symptoms.

As used herein, treatment means any manner in which the symptoms of a condition, disorder or disease are ameliorated or otherwise beneficially altered. Treatment also encompasses any pharmaceutical use of the viruses described and provided herein.

As used herein, amelioration or alleviation of symptoms associated with a disease refers to any lessening, whether permanent or temporary, lasting or transient of symptoms that can be attributed to or associated with a disease. Similarly, amelioration or alleviation of symptoms associated with administration of a virus refers to any lessening, whether permanent or temporary, lasting or transient of symptoms that can be attributed to or associated with an administration of the virus for treatment of a disease.

As used herein, an effective amount of a virus or compound for treating a particular disease is an amount that is sufficient to ameliorate, or in some manner reduce the symptoms associated with the disease. Such an amount can be administered as a single dosage or can be administered according to a regimen, whereby it is effective. The amount can cure the disease but, typically, is administered in order to ameliorate the symptoms of the disease. Repeated administration can be required to achieve the desired amelioration of symptoms.

As used herein, an effective amount of a therapeutic agent for control of viral titer in a patient is an amount that is sufficient to prevent a virus introduced to a patient for treatment of a disease from overwhelming the patient's immune system such that the patient suffers adverse side effects due to virus toxicity or pathogenicity. Such side effects can include, but are not limited to fever, abdominal pain, aches or pains in muscles, cough, diarrhea, or general feeling of discomfort or illness that are associated with virus toxicity and are related to the subject's immune and inflammatory responses to the virus. Side effects or symptoms can also include escalation of symptoms due to a systemic inflammatory response to the virus, such as, but not limited to, jaundice, blood-clotting disorders and multiple-organ system failure. Such an amount can be administered as a single dosage or can be administered according to a regimen, whereby it is effective. The amount can prevent the appearance of side effects but, typically, is administered in order to ameliorate the symptoms of the side effects associated with the virus and virus toxicity. Repeated administration can be required to achieve the desired amelioration of symptoms.

As used herein, an in vivo method refers to a method performed within the living body of a subject.

As used herein, a subject includes any animal for whom diagnosis, screening, monitoring or treatment is contemplated. Animals include mammals such as primates and domesticated animals. An exemplary primate is human. A patient refers to a subject such as a mammal, primate, human, or livestock subject afflicted with a disease condition or for which a disease condition is to be determined or risk of a disease condition is to be determined.

As used herein, cancer is a term for diseases caused by or characterized by any type of malignant tumor, including metastatic cancers, lymphatic tumors, and blood cancers. Exemplary cancers include, but are not limited to: leukemia, lymphoma, pancreatic cancer, lung cancer, ovarian cancer, breast cancer, cervical cancer, bladder cancer, prostate cancer, glioma tumors, adenocarcinomas, liver cancer and skin cancer. Exemplary cancers in humans include a bladder tumor, breast tumor, prostate tumor, basal cell carcinoma, biliary tract cancer, bladder cancer, bone cancer, brain and CNS cancer (e.g., glioma tumor), cervical cancer, choriocarcinoma, colon and rectum cancer, connective tissue cancer, cancer of the digestive system; endometrial cancer, esophageal cancer; eye cancer; cancer of the head and neck; gastric cancer; intra-epithelial neoplasm; kidney cancer; larynx cancer; leukemia; liver cancer; lung cancer (e.g. small cell and non-small cell); lymphoma including Hodgkin's and Non-Hodgkin's lymphoma; melanoma; myeloma, neuroblastoma, oral cavity cancer (e.g., lip, tongue, mouth, and pharynx); ovarian cancer; pancreatic cancer, retinoblastoma; rhabdomyosarcoma; rectal cancer, renal cancer, cancer of the respiratory system; sarcoma, skin cancer; stomach cancer, testicular cancer, thyroid cancer; uterine cancer, cancer of the urinary system, as well as other carcinomas and sarcomas.

As used herein, the term “malignant,” as it applies to tumors, refers to primary tumors that have the capacity of metastasis with loss of growth control and positional control.

As used herein, metastasis refers to a growth of abnormal or neoplastic cells distant from the site primarily involved by the morbid process.

As used herein, proliferative disorders include any disorders involving abnormal proliferation of cells, such as, but not limited to, neoplastic diseases. As used herein, a method for treating or preventing neoplastic disease means that any of the symptoms, such as the tumor, metastasis thereof, the vascularization of the tumors or other parameters by which the disease is characterized are reduced, ameliorated, prevented, placed in a state of remission, or maintained in a state of remission. It also means that the indications of neoplastic disease and metastasis can be eliminated, reduced or prevented by the treatment. Non-limiting examples of the indications include uncontrolled degradation of the basement membrane and proximal extracellular matrix, migration, division, and organization of the endothelial cells into new functioning capillaries, and the persistence of such functioning capillaries.

As used herein, a prodrug is a compound that, upon in vivo administration, is metabolized or otherwise converted to the biologically, pharmaceutically or therapeutically active form of the compound. To produce a prodrug, the pharmaceutically active compound is modified such that the active compound is regenerated by metabolic processes. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

As used herein, an anti-cancer agent or compound (used interchangeably with “anti-tumor or anti-neoplastic agent”) refers to any agents, or compounds, used in anti-cancer treatment. These include any agents, when used alone or in combination with other compounds, that can alleviate, reduce, ameliorate, prevent, or place or maintain in a state of remission of clinical symptoms or diagnostic markers associated with neoplastic disease, tumors and cancer, and can be used in methods, combinations and compositions provided herein. Exemplary anti-cancer agent agents include, but are not limited to, the viruses provided herein used singly or in combination and/or in combination with other anti-cancer agents, such as cytokines, growth factors, hormones, photosensitizing agents, radionuclides, toxins, anti-metabolites, signaling modulators, anti-cancer antibiotics, anti-cancer antibodies, anti-cancer oligopeptides, angiogenesis inhibitors, radiation therapy, hypothermia therapy, hyperthermia therapy, laser therapy, chemotherapeutic compounds, or a combination thereof.

Chemotherapeutic compounds include, but are not limited to platinum; platinum analogs anthracenediones; vinblastine; alkylating agents; alkyl sulfonates; aziridines; ethylenimines and methylamelamines; nitrosureas; antibiotics; anti-metabolites; folic acid analogues; androgens; anti-adrenals; folic acid replenisher; aminolevulinic acid; amsacrine; bestrabucil; bisantrene; edatraxate; defofamine; demecolcine; diaziquone; elformithine; elliptinium acetate; etoglucid; gallium nitrate; substituted ureas; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; anti-cancer polysaccharides; polysaccharide-K; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; cytosine arabinoside; cyclophosphamide; thiotepa; taxoids, such as paclitaxel and doxetaxel; chlorambucil; gemcitabine; 6-thioguanine; mercaptopurine; methotrexate; etoposide (VP-16); ifosfamide; mitomycin C; vincristine; vinorelbine; navelbine; novantrone; teniposide; daunomycin; aminopterin; xeloda; ibandronate; CPT11; topoisomerase inhibitor RFS 2000; difluoromethylornithine (DMFO); retinoic acid; esperamicins; capecitabine; methylhydrazine derivatives; and pharmaceutically acceptable salts, acids or derivatives of any of the above. Chemotherapeutic compounds also include, but are not limited to, adriamycin, non-sugar containing chloroethylnitrosoureas, 5-fluorouracil, bleomycin, doxorubicin, taxol, fragyline, Meglamine GLA, valrubicin, carmustaine and poliferposan, MM1270, BAY 12-9566, RAS farnesyl transferase inhibitor, farnesyl transferase inhibitor, MMP, MTA/LY231514, LY264618/Lometexol, Glamolec, CI-994, TNP-470, Hycamtin/Topotecan, PKC412, Valspodar/PSC833, Novantrone/Mitroxantrone, Metaret/Suramin, Batimastat, E7070, BCH-4556, CS-682, 9-AC, AG3340, AG3433, Incel/VX-710, VX-853, ZD0101, IS1641, ODN 698, TA 2516/Marmistat, BB2516/Marmistat, CDP 845, D2163, PD183805, DX8951f, Lemonal DP 2202, FK 317, Picibanil/OK-432, AD 32/Valrubicin, Metastron/strontium derivative, Temodal/Temozolomide, Evacet/liposomal doxorubicin, Yewtaxan/Placlitaxel, Taxol®/Paclitaxel, Xeload/Capecitabine, Furtulon/Doxifluridine, Cyclopax/oral paclitaxel, Oral Taxoid, SPU-077/Cisplatin, HMR 1275/Flavopiridol, CP-358 (774)/EGFR, CP-609 (754)/RAS oncogene inhibitor, BMS-182751/oral platinum, UFT(Tegafur/Uracil), Ergamisol/Levamisole, Eniluracil/776C85/5FU enhancer, Campto/Levamisole, Camptosar/Irinotecan, Tumodex/Ralitrexed, Leustatin/Cladribine, Paxex/Paclitaxel, Doxil/liposomal doxorubicin, Caelyx/liposomal doxorubicin, Fludara/Fludarabine, Pharmarubicin/Epirubicin, DepoCyt, ZD1839, LU 79553/Bis-Naphtalimide, LU 103793/Dolastain, Caetyx/liposomal doxorubicin, Gemzar/Gemcitabine, ZD 0473/Anormed, YM 116, Iodine seeds, CDK4 and CDK2 inhibitors, PARP inhibitors, D4809/Dexifosamide, Ifes/Mesnex/Ifosamide, Vumon®/Teniposide, Paraplatin/Carboplatin, Plantinol/cisplatin, Vepeside/Etoposide, ZD 9331, Taxotere/Docetaxel, prodrug of guanine arabinoside, Taxane Analog, nitrosoureas, alkylating agents such as melphelan and cyclophosphamide, Aminoglutethimide, Anastrozole, Asparaginase, Busulfan, Carboplatin, Chlorombucil, Cladribine, Cytarabine HCl, Dactinomycin, Daunorubicin HCl, Denileukin diftitox, Estramustine phosphate sodium, Etoposide (VP16-213), Exemestane, Floxuridine, Fluorouracil (5-FU®), Flutamide, Hydroxyurea (hydroxycarbamide), Ifosfamide, Interferon Alfa-2a, Interferon Alfa-2b, Interferon Gamma-1b, Letrozole, Leuprolide acetate (LHRH-releasing factor analogue), Lomustine (CCNU), Mechlorethamine HCl (nitrogen mustard), Megestrol, Mercaptopurine, Mesna, Mitotane (o.p′-DDD), Mitoxantrone HCl, Octreotide, Pegaspargase, Plicamycin, Procarbazine HCl, Streptozocin, Tamoxifen citrate, Thioguanine, Thiotepa, Tretinoin, Vinblastine sulfate, Amsacrine (m-AMSA), Azacitidine, Erythropoietin, Hexamethylmelamine (HMM), Interleukin 2, Mitoguazone (methyl-GAG; methyl glyoxal bis-guanylhydrazone; MGBG), Pentostatin (2′deoxycoformycin), Semustine (methyl-CCNU), Teniposide (VM-26®), Vindesine sulfate, Altretamine, Carmustine, Estramustine, Gemtuzumab ozogamicin, Idarubicin, Ifosphamide, Isotretinoin, Leuprolide, Melphalan, Testolactone, Uracil mustard, and the like. Also included in this definition are anti-hormonal agents that act to regulate or inhibit hormone action on tumors such as anti-estrogens, adrenocortical suppressants, antiandrogens and pharmaceutically acceptable salts, acids or derivatives of any of the above. Such chemotherapeutic compounds that can be used herein include compounds whose toxicities preclude use of the compound in general systemic chemotherapeutic methods.

As used herein the term assessing or determining is intended to include quantitative and qualitative determination in the sense of obtaining an absolute value for the activity of a product, and also of obtaining an index, ratio, percentage, visual or other value indicative of the level of the activity. Assessment can be direct or indirect. As used herein, activity refers to the in vivo activities of a compound or viruses on physiological responses that result following in vivo administration thereof (or of a composition or other mixture). Activity, thus, encompasses resulting therapeutic effects and pharmaceutical activity of such compounds, compositions and mixtures. Activities can be observed in in vitro and/or in vivo systems designed to test or use such activities.

As used herein, a vaccine refers to a composition which, upon administration to a subject, elicits an immune response in a subject to which it is administered and which protects the immunized subject against subsequent challenge by the immunizing agent or an immunologically cross-reactive agent. A vaccine can be used to enhance the immune response against a pathogen, such as a virus, that expresses the immunological agent and/or has already infected the subject. Protection can be complete or partial (i.e., a reduction in symptoms or infection as compared with an unvaccinated subject). Typically a vaccine is administered to a subject that is a mammal. An immunologically cross-reactive agent can be, for example, the whole protein (e.g., tumor antigen) from which a subunit peptide used as the immunogen is derived. Alternatively, an immunologically cross-reactive agent can be a different protein which is recognized in whole or in part by the antibodies elicited by the immunizing agent. Exemplary vaccines can be modified vaccinia viruses that express an immunologically cross-reactive agent.

As used herein, the phrase “immunoprivileged cells and tissues” refers to cells and tissues, such as solid tumors and wounded tissues, which are sequestered from the immune system.

As used herein, nanoparticle refers to a microscopic particle whose size is measured in nanometers. Often such particles in nanoscale are used in biomedical applications acting as drug carriers or imaging agents. Nanoparticles can be conjugated to other agents, including, but not limited to detectable/diagnostic agents or therapeutic agents.

As used herein, “a combination” refers to any association between two or among more items. Such combinations can be packaged as kits.

As used herein, a composition refers to any mixture. It can be a solution, a suspension, an emulsion, liquid, powder, a paste, aqueous, non-aqueous or any combination of such ingredients.

As used herein, fluid refers to any composition that can flow. Fluids thus encompass compositions that are in the form of semi-solids, pastes, solutions, aqueous mixtures, gels, lotions, creams and other such compositions.

As used herein, a kit is a packaged combination, optionally, including instructions for use of the combination and/or other reactions and components for such use.

### B. OVERVIEW

Cell compositions that are administered to a subject in cell therapy protocols have the potential to result in the formation a tumor, insofar as the cell composition can contain neoplastic cells or neoplastic progenitor cells. While the potential of cellular therapy in the treatment of diseases, disorders and injury is significant, the formation of tumors, such as teratomas, as a result of such treatment is an unacceptable outcome. Thus, provided herein are methods for administering cell compositions in cell therapy protocols, wherein development of cell therapy-associated tumors is inhibited. Such methods include the administration of an oncolytic virus before, with or after administration of the cell compositions, as described in detail below. The oncolytic virus also can be mixed with the cell composition prior to administration to the subject, to remove any neoplastic cells in the composition. Additionally, these oncolytic viruses also can be used in the treatment of existing cancers in a subject.

The cell compositions administered in the methods herein include any cell composition containing one or more types of cells. The cells contained in the cell compositions include, but are not limited to, stem cells (such as embryonic stem cells and adult stem cells), immune cells and non-immune cells. Exemplary immune cells that can be included in cell compositions for cell therapy are T lymphocytes (including Th1 cells, Th2 cells, tumor infusing lymphocytes (TIL) and cytotoxic T cells (CTL)) antigen presenting cells (APC) (including dendritic cells (DC) and macrophages, and natural killer (NK) cells). Non-immune cells include, but are not limited to, neuronal, skin, adrenal, keratinocyte, blood, endothelial, kidney, bone, muscle, heart, retinal, pancreas and liver cells. Typically, 1×10⁵, 1×10⁶, 2×10⁶, 3×10⁶, 4×10⁶, 5×10⁶, 1×10⁷, 2×10⁷, 3×10⁷, 4×10⁷, 5×10⁷, 6×10⁷, 7×10⁷, 8×10⁷, 9×10⁷, 1×10⁸, 5×10⁸, 1×10⁹, or 5×10⁹ cells or more are administered.

### C. CELLULAR THERAPY

Cell or cellular therapy is the process of administering a cell composition to a subject in order to treat a disease, disorder or injury. The therapy can include the transplantation of cell compositions containing autologous or allogenic cells, differentiated or undifferentiated cells, pure populations or mixed, genetically modified or unmodified cells, or any combination thereof. Additionally, the cell therapy can include other agents and factors, such as chemotherapeutic agents, chemokines, cytokines and growth factors.

Cellular therapies include cellular immunotherapies. The immune system is designed to eradicate a large number of pathogens, as well as tumors, with minimal immunopathology. When the immune system becomes defective, however, numerous disease states result. One of the aims of immunotherapy is to enhance the cellular immune response in diseases characterized by immunosuppression and suppress the cellular immune response in subjects with diseases characterized by an overactive cellular immune response. In other instances, cellular immunotherapy is used to augment a healthy immune system.

Cellular therapies also include cell replacement therapies of non-immune cells. Such therapies can be used in the treatment of degenerative diseases or disorders, and injury, in which one or more cell populations or tissues are damaged or dysfunctional. Such cellular therapies include the administration of undifferentiated cells, such as stem cells, including embryonic stem cells, adult stem cells, as well as differentiated cells, such as, for example, neuronal, skin, adrenal, keratinocyte, blood, endothelial, kidney, bone, muscle, heart, retinal, pancreas and liver cells.

Any type of mammalian cell can be included in the cell compositions administered to a subject in a cellular therapy protocol, including, but not limited to, stem cells, immune cells and non-immune cells. Exemplary immune cells that can be included in cell compositions for cell therapy are T lymphocytes (including Th1 cells, Th2 cells, tumor infusing lymphocytes (TIL) and cytotoxic T cells (CTL)) antigen presenting cells (APC) (including dendritic cells (DC) and macrophages, and natural killer (NK) cells). Non-immune cells include, but are not limited to, neuronal, skin, adrenal, keratinocyte, blood, endothelial, kidney, bone, muscle, heart, retinal, pancreas and liver cells. The cells can be activated prior to administration. For example, lymphokine-activated killer (LAK) cells are NK cells stimulated to kill tumor cells (U.S. Pat. No. 4,690,915; Clark et al., (1990) Cancer Res 50:7343-7350), and can be included in cell compositions for cell therapy.

Cells that have been genetically engineered or modified, such as to express one or more heterologous genes also are used in cell therapy. For example, cells can be engineered to express growth factors, cytokines, growth factor receptors, cytokine receptors, tumor antigen-specific receptors, suicide molecules, and detectable proteins (see e.g. June et al., (2007) 117:1466-1476). Cells also can be infected with replication competent or replication deficient viruses, including adenovirus, reovirus, vaccinia virus, herpes virus and parapox virus also can be included in the cell compositions for use in cell therapy. In some examples, the virus is a gene therapy vector.

1. Bone Marrow Transplant

Bone marrow transplant (BMT) is an example of cell therapy that had been widely used for many years for the treatment of several cancers, including leukemia, aplastic anemia, lymphomas such as Hodgkin's disease, multiple myeloma, neuroblastoma, immune deficiency disorders and some solid tumors such as breast and ovarian cancer. Bone marrow transplants can be autologous, syngeneic or allogeneic. Bone marrow contains three types of stem cells; hematopoietic stem cells (HSCs), mesenchymal stem cells (MSCs) and endothelial stem cells. Thus, BMT is a type of stem cell therapy. Bone marrow transplant typically involves treatment of patients with high dose (myeloablative) chemotherapy and/or radiation. This myeloablative conditioning results in destruction of the bone marrow leading to the loss of a functioning immune system. The subjects are then administered cells from the donor bone marrow intravenously to replace the destroyed bone marrow and restore immune function. In some examples, non-myeloablative bone marrow transplants are performed.

The ability of myeloablative conditioning followed by allogeneic BMT to cure certain hematological malignancies is widely recognized. The anti-tumor effect mediated by the allogeneic cell transplant is known as the graft vs. tumor (GVT) effect (also called the graft vs. leukemia effect and the graft vs. malignancy effect and the graft vs. myeloma effect). GVT activity after allogeneic cell therapy is known to be effective in treating several cancers, including lymphoid leukemias (Rondon et al. (1996) Bone Marrow Transplant 18:669-672), multiple myeloma (Tricot et al. (1996) Blood 87:1196-1198) and breast cancer (Eibl et al. (1996) Blood 88:1501-8).

2. T Cell Therapy

Cell therapy includes T cell therapy (also called T adoptive therapy), in which one or more populations of T cells are administered to a subject, typically for the treatment of cancer (June et al., (2007) J Clin Invest 117:1466-1476). Such methods often involve the ex vivo activation and expansion of T-cells. In some instances, cell therapy involves the removal of immune cells from a subject, ex vivo processing (i.e., activation, purification and/or expansion of the cells) and the subsequent infusion of the resulting cells back into the same subject. Lymphocytes can be isolated from tumor lesions, from lymph nodes draining the tumor or a tumor vaccine site, or from peripheral blood lymphocytes stimulated with tumor antigens in vitro.

T cell therapy includes therapy with compositions containing tumor-infiltrating lymphocytes (TIL) (U.S. Pat. No. 5,126,132, Kono et al. (2002) Clin. Cancer Res. 8:1767-1771, Dudley et al. (2005) J. Clin. Oncol. 23:2346-2357.), including CD8⁺ TIL cells (Figlin et al. (1997) Journal of Urology 158:740), cytotoxic T-cells (U.S. Pat. Nos. 6,255,073 and 5,846,827), CD4⁺ T-cells activated with anti-CD3 monoclonal antibody in the presence of IL-2 (Nishimura (1992) J. Immunol. 148:285), T-cells co-activated with anti-CD3 and anti-CD28 in the presence of IL-2 (Garlie et al. (1999) Journal of Immunotherapy 22:336), antigen-specific CD8⁺ CTL T-cells produced ex vivo and expanded with anti-CD3 and anti-CD28 monoclonal antibodies (mAb) in the presence of IL-2 (Oelke et al. (2000) Clinical Cancer Research 6:1997), and various other preparations of lymphocytes (U.S. Pat. Nos. 6,194,207, 5,443,983, 6,040,180, 5,766,920 and 6,204,058). In some examples, the effectiveness of T-cells in cell therapy protocols is enhanced when the T-cell population is in a state of maximal activation upon infusion. For example, highly activated allogenic T cells can be used in cell therapy to elicit a host vs. graft (HVG) effect, to stimulate the immune system (see, e.g. U.S. Pat. No. 7,435,592). Thus, included among the cell compositions containing T cells that are useful for cell therapy are compositions containing activated T cells.

3. Stem Cell Therapy

While stem cells traditionally have been used in the treatment of many cancers, such as in immuno-reconstitution following cancer development of cancer treatments, their role in the generation and/or recurrence of cancers is of increasing concern. For example, a body of evidence indicates that a small population of stem cells exists in a tumor. The cells are called cancer stem cells, and are thought to be important for cancer growth and recurrence. Thus, eradication of these neoplastic stem cells is a goal in any cancer treatment.

A further example of the potential tumorogenic nature of stem cells is seen in the development of stem-cell derived tumors following stem cell implantation or engraftment. Because of their self-renewing capacity and multilineage potential, stem cells are viewed as a valuable tool in cell-replacement therapy. However, this self-renewing capacity can become detrimental in instances where the stem cells rapidly differentiate in an uncontrolled manner to form benign or malignant tumors. For example, some embryonic stem cells form teratomas following implantation in both immunocompetent and immunocompromised animals.

a. Stem Cells

Stem cells can be divided into three main categories. Embryonic stem cells (ESCs) originate from the inner cell mass of the blastocyst and have unlimited self-renewing capacity and multilineage potential. Germinal stem cells are derived from the primary germinal layers of the embryo and differentiate into progenitor cells to produce specific organ cells. Somatic or adult stem cells (ASCs) are progenitor cells that exist in mature tissue and are less totipotent than ESCs. ASCs include, but are not limited to, hematopoietic stems cells (HSCs) from bone marrow, and other primitive progenitor cells such as mesenchymal stem cells (MSCs) and multipotent adult progenitor cells (PAPCs).

Because of their unique regenerative potential to differentiate into any cell type, ESCs are of particular interest for treating diseases and conditions in which damaged or destroyed cell populations or tissues need to be repaired or regenerated to restore function. Included among these diseases and conditions are, for example, degenerative diseases or conditions and acute or chronic injuries in which one or more cell populations are defective or have been depleted or destroyed. Engraftment of ESCs can provide a source of healthy cells of the desired phenotype to replace the defective or destroyed cells. Once the ESCs are implanted or engrafted into the patient, such as at the location of cell or tissue damage, the ESCs can differentiate into the desired cell or tissue type based on the physical and chemical signals in the local extracellular microenvironment. Engrafted stem cells also can have indirect therapeutic effects, such as the promotion of differentiation of endogenous cells, and immunomodulatory effects as a result of the production of soluble factors.

Other stem cells, including ASCs, also are used in stem cell therapy. ASCs can be isolated from, for example, bone marrow, adipose tissues, and peripheral blood. Because ASCs include HSCs, they can be of particular use in hematopoietic cell transplantation, such as in leukemias and following high dose chemotherapy to restore bone marrow and the immune system (see e.g., Edwards (2004) Reprod. Biomed. Online 9:541-583). ASCs also can be used in tissue regeneration, including non-hematopoietic tissue regeneration (see e.g., Serakinci et al., (2006) Eur J Cancer 42:1243-1246, Ting et al., (2008) Crit Rev Oncol Hematol 65:81-93).

Any stem cell can be used in stem cell therapy, where that stem cell has the potential to differentiate into the particular cell or tissue type of interest. In some instances, partial differentiation is induced in vitro prior to administration to a subject in a stem cell therapy protocol. This partial differentiation can be directed by, for example, the addition or removal of appropriate growth factors during stem cell culture, to produce cells of a particular lineage (see e.g. Murry, et al., (2008) Cell 132:661-680, Irion et al. (2009) Cold Spring Harb Symp Quant Biol. 2009 Mar. 27).

Embryonic Stem Cells

Embryonic stem cells (ESCs) are ideal candidates for therapeutic purposes due to their higher totipotency and indefinite life span compared to adult stem cells. ESCs can be kept undifferentiated in culture or be differentiated to tissues representing all three germ layers, both in vivo and in vitro. ESCs typically are derived from embryos cultured to the blastocyst stage using methods well known in the art (see e.g., Thomson et al., (1998) Science 282:1145-1147, U.S. Pat. Nos. 5,843,780, 5,914,268, 7,029,913), but also can be produced by other methods such as somatic cell nuclear transfer (Byrne et al. (2007) Nature 450:497-502). ESCs can be cultured in an undifferentiated state using methods well know in the art (see e.g. U.S. Pat. No. 7,432,104 and Hoffman et al., (2005) Nat Biotech 6:699-708). For example, feeder layers, such as fibroblast feeders, typically are used, although feeder free systems also have been developed (Rosler et al. (2004) Dev. Dynamics 229:259-274).

Human ESC express some of the classical markers of pluripotent cells such as OCT4, alkaline phosphatase and show high levels of telomerase activity (Thomson et al., (1998) Science 282:1145-1147, Reubinoff et al., (2000) Nature Biotechnology 18:399-404). Human ESC also can express a number of other markers, including CD9, Sox2, Thy1, major histocompatibility complex class 1, SSEA-4, TRA-1-60, TRA-1-81, AC133, c-kit and flt3 (Henderson et al. (2002) Stem Cells 20:329-337, Hoffman et al. (2005) Nat Biotech 6:699-708).

ESCs cells have the potential to differentiate into nearly all cell types of the body. In vitro they are able to generate embryoid bodies (structures with three germ layers formed by pluripotent hES cells grown in three-dimensional culture which express marker genes of all three germ layers and for different cell types. ESCs can differentiate into, for example, neuronal, skin, adrenal, keratinocyte, blood, endothelial, kidney, bone, muscle, heart, pancreas and liver cells (for review, see e.g. Stojkovic et al., (2004) Reproduction 128:259-267) and Hoffman et al. (2005 Nat Biotech 6:699-708).

Compositions containing ESCs can, therefore, be used to treat conditions amenable to ESC therapy, such as degenerative conditions or injuries in which replacement and regeneration of cells is required. ESC from humans (see e.g., Thomson et al., (1998) Science 282:1145-1147, Heins et al., (2004) Stem Cells 22:367-376, Sjögren et al., (2004) Reprod Biomed Online 9(3):326-9, Hoffman et al., Nature Biotechnology, 23: 669-708, U.S. Pat. No. 6,875,607), mice (se e.g. U.S. Pat. No. 6,190,910), rat (see e.g. Ping et al., (2008) Cell 135:1299-1310; Buehr et al., (2008) Cell 135:1287-1298) and non-human primates (Thomson et al. (1995) Proc Natl Acad Sci USA 92:7844-7848) are known in the art. Numerous human ESCs have been identified. Table 1 sets forth exemplary human ESCs, any one or more of which can be included in stem compositions for use in the treatment of conditions amenable to ESC therapy.

TABLE 1
Exemplary human ESCs

| ESC Name | Provider |
|---|---|
| ACT-14 | Advanced Cell |
| | Technology |
| AS034 | Cellartis AB |
| AS034.1 | Cellartis AB |
| AS034.2 | Cellartis AB |
| AS038 | Cellartis AB |
| AS079 | Cellartis AB |
| AS094 | Cellartis AB |
| BG01 | BresaGen, Inc. |
| BG02 | BresaGen, Inc. |
| BG03 | BresaGen, Inc. |
| BG04 | BresaGen, Inc. |
| CH01 | Cell & Gene Therapy |
| | Research Institute |
| CH02 | Cell & Gene Therapy |
| | Research Institute |
| CLS1 | Aalborg University, |
| | Denmark |
| CLS2 | Aalborg University, |
| | Denmark |
| CLS3 | Aalborg University, |
| | Denmark |
| CLS4 | Aalborg University, |
| | Denmark |
| ES01 | ES Cell International |
| ES02 | ES Cell International |
| ES03 | ES Cell International |
| ES04 | ES Cell International |
| ES05 | ES Cell International |
| ES06 | ES Cell International |
| ESM01 | Institute of Gene Biology; |
| | Russian Academy of |
| | Sciences |
| ESM02 | Institute of Gene Biology; |
| | Russian Academy of |
| | Sciences |
| ESM03 | Institute of Gene Biology; |
| | Russian Academy of |
| | Sciences |
| FC018 | Cellartis AB |
| FES 21 | University of Helsinki |
| FES 22 | University of Helsinki |
| FES 29 | University of Helsinki |
| FES 30 | University of Helsinki |
| GE01 | Geron Corporation |
| GE07 | Geron Corporation |
| GE09 | Geron Corporation |
| GE13 | Geron Corporation |
| GE14 | Geron Corporation |
| GE91 | Geron Corporation |
| GE92 | Geron Corporation |
| hES-NCL1 | University of Newcastle |
| | upon Tyne, UK |
| HS181 | Karolinska Institute |
| HS207 | Karolinska Institute |
| HUES1 | HUES Cell Facility |
| HUES10 | HUES Cell Facility |
| HUES11 | HUES Cell Facility |
| HUES12 | HUES Cell Facility |
| HUES13 | HUES Cell Facility |
| HUES14 | HUES Cell Facility |
| HUES15 | HUES Cell Facility |
| HUES16 | HUES Cell Facility |
| HUES17 | HUES Cell Facility |
| HUES2 | HUES Cell Facility |
| HUES3 | HUES Cell Facility |
| HUES4 | HUES Cell Facility |
| HUES5 | HUES Cell Facility |
| HUES6 | HUES Cell Facility |
| HUES7 | HUES Cell Facility |
| HUES8 | HUES Cell Facility |
| HUES9 | HUES Cell Facility |
| KhES-1 | Department of Stem Cell |
| | Biology, Institute for |
| | Frontier Medical Sciences, |
| | Kyoto University |
| KhES-2 | Department of Stem Cell |
| | Biology, Institute for |
| | Frontier Medical Sciences, |
| | Kyoto University |
| KhES-3 | Department of Stem Cell |
| | Biology, Institute for |
| | Frontier Medical Sciences, |
| | Kyoto University |
| MB01 | Maria Biotech Co. Ltd. |
| MB02 | Maria Biotech Co. Ltd. |
| MB03 | Maria Biotech Co. Ltd. |
| Miz-hES1 | MizMedi Hospital - Seoul |
| | National University |
| Miz-hES10 | MizMedi Hospital - Seoul |
| | National University |
| Miz-hES11 | MizMedi Hospital - Seoul |
| | National University |
| Miz-hES12 | MizMedi Hospital - Seoul |
| | National University |
| Miz-hES13 | MizMedi Hospital - Seoul |
| | National University |
| Miz-hES14 | MizMedi Hospital - Seoul |
| | National University |
| Miz-hES15 | MizMedi Hospital - Seoul |
| | National University |
| Miz-hES2 | MizMedi Hospital - Seoul |
| | National University |
| Miz-hES3 | MizMedi Hospital - Seoul |
| | National University |
| Miz-hES4 | MizMedi Hospital - Seoul |
| | National University |
| Miz-hES5 | MizMedi Hospital - Seoul |
| | National University |
| Miz-hES6 | MizMedi Hospital - Seoul |
| | National University |
| Miz-hES7 | MizMedi Hospital - Seoul |
| | National University |
| Miz-hES8 | MizMedi Hospital - Seoul |
| | National University |
| NC01 | National Centre For |
| | Biological Sciences/Tata |
| | Institute of Fundamental |
| | Research |
| NC02 | National Centre For |
| | Biological Sciences/Tata |
| | Institute of Fundamental |
| | Research |
| NC03 | National Centre For |
| | Biological Sciences/Tata |
| | Institute of Fundamental |
| | Research |
| ReliCellhES1 | Reliance Life Sciences |
| RH1 | Roslin Institute |
| RH3 | Roslin Institute |
| RH4 | Roslin Institute |
| RH5 | Roslin Institute |
| RH6 | Roslin Institute |
| RH7 | Roslin Institute |
| RL05 | Reliance Life Sciences |
| RL07 | Reliance Life Sciences |
| RL10 | Reliance Life Sciences |
| RL13 | Reliance Life Sciences |
| RL15 | Reliance Life Sciences |
| RL20 | Reliance Life Sciences |
| RL21 | Reliance Life Sciences |
| Royan H1 | Royan Institute |
| SA001 | Cellartis AB |
| SA002 | Cellartis AB |
| SA002.5 | Cellartis AB |
| SA046 | Cellartis AB |
| SA085 | Cellartis AB |
| SA111 | Cellartis AB |
| SA121 | Cellartis AB |
| SA142 | Cellartis AB |
| SA167 | Cellartis AB |
| SA181 | Cellartis AB |
| SA191 | Cellartis AB |
| SA196 | Cellartis AB |
| SA202 | Cellartis AB |
| SA203 | Cellartis AB |
| SA211 | Cellartis AB |
| SA218 | Cellartis AB |
| SA240 | Cellartis AB |
| SA279 | Cellartis AB |
| SA348 | Cellartis AB |
| SA352 | Cellartis AB |
| SA399 | Cellartis AB |
| SA611 | Cellartis AB |
| SI-100 | Stemride International |
| | Ltd. |
| SI-101 | Stemride International |
| | Ltd. |
| SI-102 | Stemride International |
| | Ltd. |
| SI-103 | Stemride International |
| | Ltd. |
| SI-104 | Stemride International |
| | Ltd. |
| SI-105 | Stemride International |
| | Ltd. |
| SI-106 | Stemride International |
| | Ltd. |
| SI-107 | Stemride International |
| | Ltd. |
| SI-108 | Stemride International |
| | Ltd. |
| SI-109 | Stemride International |
| | Ltd. |
| SI-110 | Stemride International |
| | Ltd. |
| SI-111 | Stemride International |
| | Ltd. |
| SI-114 | Stemride International |
| | Ltd. |
| SI-115 | Stemride International |
| | Ltd. |
| SI-122 | Stemride International |
| | Ltd. |
| SI-123 | Stemride International |
| | Ltd. |
| SI-124 | Stemride International |
| | Ltd. |
| SI-125 | Stemride International |
| | Ltd. |
| SI-126 | Stemride International |
| | Ltd. |
| SI-128 | Stemride International |
| | Ltd. |
| SI-130 | Stemride International |
| | Ltd. |
| SI-131 | Stemride International |
| | Ltd. |
| SI-132 | Stemride International |
| | Ltd. |
| SI-133 | Stemride International |
| | Ltd. |
| SI-134 | Stemride International |
| | Ltd. |
| SI-135 | Stemride International |
| | Ltd. |
| SI-137 | Stemride International |
| | Ltd. |
| SI-138 | Stemride International |
| | Ltd. |
| SI-139 | Stemride International |
| | Ltd. |
| SI-140 | Stemride International |
| | Ltd. |
| SI-141 | Stemride International |
| | Ltd. |
| SI-144 | Stemride International |
| | Ltd. |
| SI-145 | Stemride International |
| | Ltd. |
| SI-146 | Stemride International |
| | Ltd. |
| SI-148 | Stemride International |
| | Ltd. |
| SI-149 | Stemride International |
| | Ltd. |
| SI-15 | Stemride International |
| | Ltd. |
| SI-150 | Stemride International |
| | Ltd. |
| SI-151 | Stemride International |
| | Ltd. |
| SI-153 | Stemride International |
| | Ltd. |
| SI-154 | Stemride International |
| | Ltd. |
| SI-155 | Stemride International |
| | Ltd. |
| SI-156 | Stemride International |
| | Ltd. |
| SI-157 | Stemride International |
| | Ltd. |
| SI-158 | Stemride International |
| | Ltd. |
| SI-159 | Stemride International |
| | Ltd. |
| SI-160 | Stemride International |
| | Ltd. |
| SI-161 | Stemride International |
| | Ltd. |
| SI-162 | Stemride International |
| | Ltd. |
| SI-163 | Stemride International |
| | Ltd. |
| SI-164 | Stemride International |
| | Ltd. |
| SI-165 | Stemride International |
| | Ltd. |
| SI-167 | Stemride International |
| | Ltd. |
| SI-168 | Stemride International |
| | Ltd. |
| SI-169 | Stemride International |
| | Ltd. |
| SI-170 | Stemride International |
| | Ltd. |
| SI-171 | Stemride International |
| | Ltd. |
| SI-172 | Stemride International |
| | Ltd. |
| SI-174 | Stemride International |
| | Ltd. |
| SI-175 | Stemride International |
| | Ltd. |
| SI-176 | Stemride International |
| | Ltd. |
| SI-177 | Stemride International |
| | Ltd. |
| SI-178 | Stemride International |
| | Ltd. |
| SI-179 | Stemride International |
| | Ltd. |
| SI-18 | Stemride International |
| | Ltd. |
| SI-180 | Stemride International |
| | Ltd. |
| SI-182 | Stemride International |
| | Ltd. |
| SI-183 | Stemride International |
| | Ltd. |
| SI-184 | Stemride International |
| | Ltd. |
| SI-185 | Stemride International |
| | Ltd. |
| SI-186 | Stemride International |
| | Ltd. |
| SI-187 | Stemride International |
| | Ltd. |
| SI-188 | Stemride International |
| | Ltd. |
| SI-189 | Stemride International |
| | Ltd. |
| SI-191 | Stemride International |
| | Ltd. |
| SI-192 | Stemride International |
| | Ltd. |
| SI-193 | Stemride International |
| | Ltd. |
| SI-194 | Stemride International |
| | Ltd. |
| SI-195 | Stemride International |
| | Ltd. |
| SI-196 | Stemride International |
| | Ltd. |
| SI-197 | Stemride International |
| | Ltd. |
| SI-198 | Stemride International |
| | Ltd. |
| SI-199 | Stemride International |
| | Ltd. |
| SI-200 | Stemride International |
| | Ltd. |
| SI-201 | Stemride International |
| | Ltd. |
| SI-202 | Stemride International |
| | Ltd. |
| SI-203 | Stemride International |
| | Ltd. |
| SI-204 | Stemride International |
| | Ltd. |
| SI-205 | Stemride International |
| | Ltd. |
| SI-206 | Stemride International |
| | Ltd. |
| SI-208 | Stemride International |
| | Ltd. |
| SI-209 | Stemride International |
| | Ltd. |
| SI-21 | Stemride International |
| | Ltd. |
| SI-210 | Stemride International |
| | Ltd. |
| SI-211 | Stemride International |
| | Ltd. |
| SI-213 | Stemride International |
| | Ltd. |
| SI-214 | Stemride International |
| | Ltd. |
| SI-215 | Stemride International |
| | Ltd. |
| SI-216 | Stemride International |
| | Ltd. |
| SI-217 | Stemride International |
| | Ltd. |
| SI-221 | Stemride International |
| | Ltd. |
| SI-24 | Stemride International |
| | Ltd. |
| SI-27 | Stemride International |
| | Ltd. |
| SI-28 | Stemride International |
| | Ltd. |
| SI-31 | Stemride International |
| | Ltd. |
| SI-33 | Stemride International |
| | Ltd. |
| SI-53 | Stemride International |
| | Ltd. |
| SI-60 | Stemride International |
| | Ltd. |
| SI-62 | Stemride International |
| | Ltd. |
| SI-63 | Stemride International |
| | Ltd. |
| SI-79 | Stemride International |
| | Ltd. |
| SI-80 | Stemride International |
| | Ltd. |
| SI-81 | Stemride International |
| | Ltd. |
| SI-93 | Stemride International |
| | Ltd. |
| SI-94 | Stemride International |
| | Ltd. |
| SI-95 | Stemride International |
| | Ltd. |
| SI-96 | Stemride International |
| | Ltd. |
| SI-97 | Stemride International |
| | Ltd. |
| SI-98 | Stemride International |
| | Ltd. |
| SI-99 | Stemride International |
| | Ltd. |
| SNUhES1 | Seoul National University |
| SNUhES2 | Seoul National University |
| SNUhES3 | Seoul National University |
| TE-03 | Technion-Israel Institute |
| | of Technology |
| TE-04 | Technion-Israel Institute |
| | of Technology |
| TE-06 | Technion-Israel Institute |
| | of Technology |
| TE-07 | Technion-Israel Institute |
| | of Technology |
| TE-32 | Technion-Israel Institute |
| | of Technology |
| TE-33 | Technion-Israel Institute |
| | of Technology |
| TE-62 | Technion-Israel Institute |
| | of Technology |
| TE-72 | Technion-Israel Institute |
| | of Technology |
| UC01 | University of California at |
| | San Francisco |
| UC06 | University of California at |
| | San Francisco |
| VAL-1 | Valencia Stem Cell Bank |
| VAL-2 | Valencia Stem Cell Bank |
| VAL-3 | Valencia Stem Cell Bank |
| VAL-4 | Valencia Stem Cell Bank |
| WA01 | Wisconsin Alumni |
| | Research Foundation |
| | (WiCell Research |
| | Institute) |
| WA07 | Wisconsin Alumni |
| | Research Foundation |
| | (WiCell Research |
| | Institute) |
| WA09 | Wisconsin Alumni |
| | Research Foundation |
| | (WiCell Research |
| | Institute) |
| WA13 | Wisconsin Alumni |
| | Research Foundation |
| | (WiCell Research |
| | Institute) |
| WA14 | Wisconsin Alumni |
| | Research Foundation |
| | (WiCell Research |
| | Institute) |

Modified from stemcellcommunity.org/

ESCs can be administered as undifferentiated cells or can be partially differentiated prior to administration in cell-replacement therapies. For example, ESCs can be differentiated in vitro for 1, 2, 3, 4, 5, 6, 7, 8, 9, 10, 11, 12, 13, 14, 15, 16, 17, 18, 19, 20, 25 or 30 days or more before they are administered to a subject. Conditions and methods for differentiation of ESCs are well known in the art (see e.g. Murry et al. (2008) Cell 132:661-680, Irion et al. (2009) Cold Spring Harb Symp Quant Biol. 2009 Mar. 27). Basic methods that have been developed to promote differentiation of ESCs include, for example, the formation of three-dimensional aggregates known as embryoid bodies (EBs), the culture of ESCs as monolayers on extracellular matrix proteins, and the culture of ESCs directly on supportive stromal layers. In one example of differentiation in vitro, once a population of ESCs resembling the epiblast of the embryo is formed, the population can be induced with, for example, Wnt, activin, BMP4, or serum, to generate a primitive streak (PS)-like population. If these pathways are not activated, the epiblast population can differentiate into the ectoderm lineage, with the potential to differentiate into neurons. Ectoderm differentiation can be blocked by BMP, Wnt, and activin signaling, which instead results in the formation of a PS cell population. Posterior PS cells expressing Foxa2^{low/-} produce Flk-1⁺ mesoderm, whereas the anterior PS cells are generate Foxa2⁺ endoderm. However, these fates are not firmly established, as activin can induce endoderm from the posterior PS population. Flk-1 mesoderm can be induced to form cells of the hematopoietic and vascular lineage by the addition of VEGF to culture. Foxa2⁺ endoderm can be induced to form cells of either the hepatocyte or pancreatic lineages, by the addition of BMP4 and bFGF or retinoic acid, respectively (Murry et al. (2008) Cell 132:661-680).

b. Conditions Amenable to Stem Cell Therapy

Conditions amenable to stem cell therapy include any in which one or more cell populations are defective or have been depleted or destroyed. Such conditions include degenerative disorders or conditions and acute or chronic injuries. Once the stem cells are implanted or engrafted into the patient, such as at the location of cell or tissue damage or dysfunction, the stem cells can differentiate into the desired cell or tissue type based on the physical and chemical signals in the local extracellular microenvironment, thereby replacing the destroyed or defective cells with functional healthy cells. Exemplary conditions include, but are not limited to, cancer, cardiovascular disease, diabetes, spinal cord injury, neurodegenerative disease, traumatic brain injury, Alzheimer's disease, Parkinson's disease, multiple sclerosis (MS), Amyotrophic lateral sclerosis (ALS), Duchenne Muscular Dystrophy, muscle damage or dystrophy, stroke, burns, lung disease, retinal disease, kidney disease, osteoarthritis, and rheumatoid arthritis.

In one example, stem cell therapy is used to treat cardiovascular disease. Acute or chromic cardiovascular disease can deprive heart tissue of oxygen, thereby killing cardiac muscle cells (cardiomyocytes). This loss triggers a cascade of detrimental events, including formation of scar tissue, an overload of blood flow and pressure capacity, the overstretching of viable cardiac cells attempting to sustain cardiac output, leading to heart failure, and eventual death. Damaged heart muscle tissue can be repaired by implantation of stem cells. In some examples, the stem cell compositions can be administered by an intracoronary route, such as using intracoronary catheterization techniques. The stem cells may can be administered intramyocardially, transendocardially or trans-epicardially. In other examples, in which the patient has a myocardial infarct, the stem cells are delivered to the border area of the infarct.

Stem cells also can be used to treat diabetes mellitus. Type 1 diabetes results from autoimmune-mediated destruction of insulin-secreting β cells in the islets of Langerhans of the pancreas. Type 2 diabetes results from systemic insulin resistance and reduced insulin secretion by pancreatic β cells. Stem cells have been shown in vitro to differentiate into insulin-producing cells (see e.g. Schuldiner et al. (2000) Proc. Natl. Acad. Sci. USA. 97:11307-11312; Guo et al., (2009) Endocr Rev 30:214-227). Thus, stem cells, including ESCs and ASCs, and their derivatives, such as partially differentiated stem cells, can be used in stem cell therapy for regeneration of pancreatic β cells.

In other examples, stem cell therapy is used to treat spinal cord injury. Spinal cord injury typically results in permanent disability. Insult to the spinal cord initiates a cascade of concomitant events that include anatomical, physiological, and neurochemical changes often leading to neuronal cell death and syrinx formation. Loss of motor function, altered sensory function, and/or development of chronic or neuropathic pain can then develop depending on the location and severity of injury. Animal studies have indicated that stem cells can repair damage to the spinal cord and restore mobility. For example, embryonic stem cell-derived neural stem cells (NSCs) administered to mice with spinal cord injury resulted in recovery of locomotor function (Kimura et al., (2005) Neurol Res 812-819, Cummings et al., (2006) Neurol Res (2006) 25:474-481). In other studies, embryonic stem cells predifferentiated into neuronal and glial progenitors were used in rats for treatment of pain syndromes following SCI (Hendricks et al. (2006) Mol. Med. 12:34-46).

Parkinson's disease also is amenable to treatment by engraftment of stem cells. Parkinson's disease is a neurodegenerative disorder that results from the destruction of dopaminergic neurons within a particular region of the brain. Stem cells engrafted into subjects with Parkinson's disease in stem cell therapy can differentiate into functional dopaminergic neurons. Several studies in animal models have demonstrated the effectiveness of treating Parkinson's disease with stem cells and stem cell derivatives (i.e. stem cells that have been differentiated in vitro). For example, intra-striatum transplantation of ES-derived dopaminergic neurons was effective in treating mice in a model of Parkinson's disease (Toriumi et al., (2009) Neurol. Res 31:220-227). In another example, dopaminergic neurons generated from monkey embryonic stem cells attenuated neurological symptoms in a Parkinson primate model (Takagi et al (2005) J Clin Invest. 115:102-109).

Disorders associated with demyelination in the central nervous system, such as multiple sclerosis, also are amenable to stem cell therapy. Demyelination can occur when the oligodendrocyte or the myelin sheath it produces and maintains is the target of the disease process. Once the axons have been demyelinated, they are vulnerable to atrophy, resulting in neurologic defects. Multiple sclerosis (MS) is an autoimmune disease in which the fatty myelin sheath that wraps around nerve cells and speeds up their rate of transmission is targeted by the immune system. Stem cell therapy can effect remyelination of the axons. For example, autologous non-myeloablative haemopoietic stem cell transplantation in human subjects with MS reversed neurological symptoms (Burt et al., (2009) 8:244-253). This can occur via one or more mechanisms, including, but not limited to, producing soluble immunomodulatory factors, direct cell replacement by differentiating into neural and glial cells in the lesion, and indirect action by promoting neural and glial differentiation of endogenous cells (Yang et al., (2009) J. Neurol Sci 276:1-5).

The stem cells can be implanted locally at the site of cell damage or dysfunction, or systemically. Exemplary routes of administration of stem cell compositions in stem cell therapy include, but are not limited to, intravenous, intramuscular, intradermal, intraperitonal, intracoronary, intramyocardial, transendocardial, trans-epicardial, intraspinal, intra-arterial, intra-striatum, intra-tumoral, topical, transdermal, rectal or sub-epidermal routes. The most suitable route for administration will vary depending upon the disorder or condition to be treated, for example the location of cell damage or dysfunction. For example, stem cells can be administered intra-arterially or intra-spinally at the site of injury for the treatment of spinal cord injury. In other examples, stem cell compositions can be administered by an intracoronary, intramyocardial, transendocardial or trans-epicardial route for the treatment of cardiovascular disease.

c. Stem Cell-Associated Tumors

As discussed above, the ability of stems cells to differentiate is one of the characteristics that makes them appealing for cell-replacement therapy. However, it is this same characteristic that is of significant concern in the clinic. Stem cells that are engrafted into a patient in cell-replacement therapy have the potential not only to differentiate into the desired cell or tissue type, but also have the potential to differentiate in an uncontrolled manner to form benign or malignant tumors.

In addition to their ability to spontaneously differentiate in vitro in the form of embryoid bodies (EBs), ESCs also can spontaneously differentiate following transplantation in vivo, rapidly forming of tumors called teratomas. These are benign masses of haphazardly differentiated tissues. Teratomas also can appear spontaneously in humans and in mice. Teratomas that arise from ESC transplantation can be immature or mature. Mature teratomas contain only mature, well differentiated tissues, while immature teratomas contain tissues of a more embryonic, less-differentiated mature. When the tumors contain a core of malignant undifferentiated cells, they are considered teratocarcinomas. These malignant undifferentiated cells are termed embryonic carcinoma (EC), and are considered the malignant counterparts of embryonic stem cells (Blum et al., (2008) Adv. Cancer. Res 133-158).

The injection of pluripotent ESC or ESC-derived precursor cells in both immunocompetent and immunosuppressed rodents can lead to the development of teratomas or teratocarcinomas (see e.g. Bjorklund et al. (2002) Proc Natl Acad Sci USA 99: 2344-2349, Isacson et al. (1995) Nat Med 1: 1189-1194, Reubinoff et al., (2000) Nat Biotechnol 18: 399-404). The teratomas can form in healthy animals as well as those with a disease or condition for which ESCs are being administered for treatment. For example, ESCs that were transplanted intramyocardially into healthy mice formed extracardic teratomas (Cao et al., (2007) Stem Cells Dev. 16:883-891). In another example, ESCs engrafted into the spinal chord of rats can form teratomas at the site of engraftment. This can occur in healthy rats and also rats with acute spinal injury or spinal ischemia (see Example 3, below). In a further example, teratomas formed following transplantation of ESCs in a rat model of Parkinson's disease (Brederlau et al., (2006) Stem Cells 24:1433-1440). Tumor formation also was observed following ESC transplantation in an experimental traumatic brain injury model in rats (Reiss et al., (2007) J. Neurotrauma 24:216-225)

There is evidence to suggest that culture-adapted human ESCs typically give rise to teratomas of a less-differentiated nature, while nonadapted human ESCs give rise to mature teratomas (Blum et al., (2008) Adv. Cancer. Res 133-158). Further, ESCs appear to more prone to generate tumors when implanted into the same species from which they were derived (Erdo et al. (2003) J Cereb Blood Flow Metab 23: 780-78). Although the exact mechanism by which ESCs become tumorogenic and form teratomas is unknown, recent reports implicate the BIRC5 gene this process. (Blum et al. (2009) Nature Biotechnol 27:281-287). BIRC5 codes for survivin, a protein which both inhibits apoptosis and regulates cell division.

There is little data regarding the formation of tumors following stem cell engraftment in humans, as such studies typically are not feasible. However, in one recent clinical example, a patient with ataxia telangiectasia (AT) was treated with intracerebellar and intrathecal injection of human fetal neural stem cells. Four years after the first treatment, the patient was diagnosed with a multifocal brain tumor that was shown to be derived from the donor stem cells. (Amariglio et al. (2009) PLoS Medicine 6:221-231).

While the potential of stem cells for cell-replacement therapy is significant, the formation of stem-cell derived tumors as a result of such treatment is an unacceptable outcome. Thus, provided herein are methods for administering stem cell compositions, wherein development of stem cell-associated tumors is inhibited. Such methods include the administration of an oncolytic virus before, with or after administration of stem cell compositions, as described in detail below. The oncolytic virus also can be mixed with the stem cell composition prior to administration to the subject, to remove any neoplastic cells in the composition.

### D. THERAPEUTIC AND DIAGNOSTIC METHODS FOR THE TREATMENT AND PREVENTION OF STEM CELL-DERIVED TUMORS

Provided are therapeutic methods for treating and/or preventing the formation of stem cell-derived tumors and metastases. Such methods provide for the safe administration of stem cell therapies and can inhibit or treat complications of tumor formation (e.g. teratoma or teratocarcinoma formation) that result from administration of stem cell compositions to a subject for therapy of a disease or disorder. Exemplary diseases or disorders for which the methods provided herein can be employed include, but are not limited to, cardiovascular disease, cancer, diabetes, spinal cord injury, neurodegenerative disease, Alzheimer's disease, Parkinson's disease, multiple sclerosis, Amyotrophic lateral sclerosis, Duchenne Muscular Dystrophy, muscle damage or dystrophy, stroke, burns, lung disease, retinal disease, kidney disease, osteoarthritis, and rheumatoid arthritis.

The therapeutic methods provided herein include, but are not limited to, administering a stem Cell therapy, such as the administration of a stem cell composition (e.g., an embryonic stem cell composition) to a subject, in combination with an oncolytic virus provided herein to prevent tumor formation or to treat a tumor resulting from or generated by the administered stem cell composition. In some examples, the virus is administered to a subject having a tumor, where the tumor is caused by the stem cell therapy. In other examples, the virus is administered to a subject who is receiving or has received a stem cell therapy for the prevention of tumor formation.

The administered viruses are typically attenuated viruses that preferentially accumulate in neoplastic cells, including tumors or metastases. Generally, the administered viruses are replication competent viruses and have the ability to preferentially replicate in tumor cells and/or lyse the tumor cells (i.e., oncolytic viruses). In some examples, administration of a virus provided herein results in elimination of neoplastic cells or neoplastic progenitor cells in the stem cell composition that have the ability to form a tumor. The oncolytic viruses provided herein can eliminate neoplastic cells or neoplastic progenitor cells in a stem cell composition either in vitro or in vivo. In some examples, administration of a virus provided herein results in a slowing the growth of a stem cell-derived tumor. In other examples, the administration of a virus provided herein results in a decrease in the volume of the stem cell-derived tumor. In other examples, the administration of a virus provided herein results in elimination of the stem cell-derived tumor.

In some examples, the virus is administered concurrently with administration of a stem cell composition. In other examples, the virus is administered at a selected time point following administration of a stem cell composition. In other examples, the stem cell composition is pretreated with the virus prior to administration of the stem cell composition to the subject. In some examples, the stem cell composition is pretreated with the virus prior to administration of the stem cell composition to the subject and the same or different oncolytic virus also is administered concurrently with or subsequent to the administration of the pre-treated stem cell composition.

The viruses can be administered for diagnosis and/or therapy of subjects, such as, but not limited to humans and other mammals, including rodents, dogs, cats, primates, or livestock.

1. Viruses for Use in the Methods Provided

Viruses for use in the methods provided herein typically are replication competent viruses that can selectively infect neoplastic cells and cause lysis of the infected cell (i.e. oncolytic viruses). Such viruses can accumulate in immunoprivileged cells or immunoprivileged tissues, including tumors and/or metastases, and can infect neoplastic cells of a stem cell composition. Accordingly, these viruses can be used to eliminate neoplastic cells from stem cell compositions, including implanted stem cell compositions, and stem cell-derived tumors. In some examples, the modified viruses have an ability to activate an immune response against tumor cells without aggressively killing the tumor cells.

Viruses for use in the methods provided herein typically are modified viruses, which are modified relative to the wild-type virus. Such modifications of the viruses provided can enhance one or more characteristics of the virus. Such characteristics can include, but are not limited to, attenuated pathogenicity, reduced toxicity, preferential accumulation in tumor, increased ability to activate an immune response against tumor cells, increased immunogenicity, increased or decreased replication competence, and ability to express additional exogenous proteins, and combinations thereof. For examples, the viruses can be modified to express one or more detectable gene products, including proteins that can be used for detecting, imaging and monitoring of neoplastic cells in the stem cell composition or a stem cell-derived tumor. In other examples, the viruses can be modified to express one or more gene product for the therapy of a tumor.

Viruses for use in the methods provided herein can contain one or more additional heterologous nucleic acid molecules inserted into the genome of the virus. A heterologous nucleic acid molecule can contain an open reading frame operatively linked to a promoter for expression or can be a non-coding sequence that alters the attenuation of the virus. In some cases, the heterologous nucleic acid replaces all or a portion of a viral gene.

In some examples, the administered viruses can cause cell lysis or tumor cell death as a result of expression of an endogenous gene or as a result of an exogenous gene. Endogenous or exogenous genes can cause tumor cell lysis or inhibit cell growth as a result of direct or indirect actions, as is known in the art, including lytic channel formation or activation of an apoptotic pathway. Gene products, such as exogenous gene products can function to activate a prodrug to an active, cytotoxic form, resulting in cell death where such genes are expressed.

The administered virus can stimulate humoral and/or cellular immune response in the subject, such as the induction of cytotoxic T lymphocytes responses against the stem cell-derived tumor. For example, the virus can provide prophylactic and therapeutic effects against a tumor infected by the virus or other infectious diseases, by rejection of cells from tumors or lesions using viruses that express immunoreactive antigens (Earl et al., Science 234: 728-831 (1986); Lathe et al., Nature (London) 32: 878-880 (1987)), cellular tumor-associated antigens (Bernards et al., Proc. Natl. Acad. Sci. USA 84: 6854-6858 (1987); Estin et al., Proc. Natl. Acad. Sci. USA 85: 1052-1056 (1988); Kantor et al., J. Natl. Cancer Inst. 84: 1084-1091 (1992); Roth et al., Proc. Natl. Acad. Sci. USA 93: 4781-4786 (1996)) and/or cytokines (e.g., IL-2, IL-12), costimulatory molecules (B7-1, B7-2) (Rao et al., J. Immunol. 156: 3357-3365 (1996); Chamberlain et al., Cancer Res. 56: 2832-2836 (1996); Oertli et al., J. Gen. Virol. 77: 3121-3125 (1996); Qin and Chatterjee, Human Gene Ther. 7: 1853-1860 (1996); McAneny et al., Ann. Surg. Oncol. 3: 495-500 (1996)), or other therapeutic proteins.

In some examples, the administration of a virus provided herein causes enhancement of an anti-tumor immune response against the stem cell-derived tumor cells by release of tumor antigens upon tumor cell lysis or tumor cell death. The anti-tumor immune response induced as a result of the tumor-accumulated viruses can result in inhibition of tumor growth, decrease in tumor volume, or elimination of the tumor.

In some examples, the viruses can be modified to express one or more antigens to elicit antibody production against an expressed gene product and enhance the immune response against the infected tumor cell. The sustained release of antigen can result in an immune response by the viral-infected host, in which the host can develop antibodies against the antigen, and/or the host can mount an immune response against cells expressing the antigen, including an immune response against tumor cells. Thus, the sustained release of antigen can result in immunization against tumor cells. In some embodiments, the viral-mediated sustained antigen release-induced immune response against tumor cells can result in complete removal or killing of all tumor cells. The immunizing antigens can be endogenous to the virus, such as vaccinia antigens on a vaccinia virus used to immunize against smallpox, measles, mumps, or the immunizing antigens can be exogenous antigens expressed by the virus, such as influenza or HIV antigens expressed on a viral capsid surface. In the case of smallpox, for example, a tumor specific protein antigen can be carried by an attenuated vaccinia virus (encoded by the viral genome) for a smallpox vaccine. Thus, the viruses provided herein, including the modified vaccinia viruses can be used as vaccines.

As shown previously, solid tumors can be treated with viruses, such as vaccinia viruses, resulting in an enormous tumor-specific virus replication, which can lead to tumor protein antigen and viral protein production in the tumors (U.S. Patent Publication No. 2005/0031643). Vaccinia virus administration to mice resulted in lysis of the infected tumor cells and a resultant release of tumor-cell-specific antigens. Continuous leakage of these antigens into the body led to a very high level of antibody titer (in approximately 7-14 days) against tumor proteins, viral proteins, and the virus encoded engineered proteins in the mice. The newly synthesized anti-tumor antibodies and the enhanced macrophage, neutrophils count were continuously delivered via the vasculature to the tumor and thereby provided for the recruitment of an activated immune system against the tumor. The activated immune system then eliminated the foreign compounds of the tumor including the viral particles. This interconnected release of foreign antigens boosted antibody production and continuous response of the antibodies against the tumor proteins to function like an autoimmunizing vaccination system initiated by vaccinia viral infection and replication, followed by cell lysis, protein leakage and enhanced antibody production. Thus, the viruses provided herein and the viruses generated using the methods provided herein can be administered in a complete process that can be applied to therapy or prevention of tumors resulting from stem cell therapies.

a. Exemplary Viruses

Exemplary viruses for use in the methods provided herein include cytoplasmic viruses, which do not require entry of viral nucleic acid molecules in to the nucleus of the host cell during the viral life cycle. A variety of cytoplasmic viruses are known, including, but not limited to, pox viruses, African swine flu family viruses, and various RNA viruses such as picornaviruses, caliciviruses, togaviruses, coronaviruses and rhabdoviruses. Exemplary cytoplasmic viruses provided herein are viruses of the poxvirus family, including orthopoxviruses. Exemplary of poxviruses provided herein are vaccinia viruses,

i. Poxviruses

In some examples, the virus provided herein is selected from the poxvirus family. Poxviruses include Chordopoxyiridae such as orthopoxvirus, parapoxvirus, avipoxvirus, capripoxvirus, leporipoxvirus, suipoxvirus, molluscipoxvirus and yatapoxvirus, as well as Entomopoxyirinae such as entomopoxvirus A, entomopoxvirus B, and entomopoxvirus C. One skilled in the art can select a particular genera or individual chordopoxyiridae according to the known properties of the genera or individual virus, and according to the selected characteristics of the virus (e.g., pathogenicity, ability to elicit an immune response, preferential tumor localization), the intended use of the virus, the tumor type and the host organism. Exemplary chordopoxyiridae genera are orthopoxvirus and avipoxvirus.

Avipoxviruses are known to infect a variety of different birds and have been administered to humans. Exemplary avipoxviruses include canarypox, fowlpox, juncopox, mynahpox, pigeonpox, psittacinepox, quailpox, peacockpox, penguinpox, sparrowpox, starlingpox, and turkeypox viruses.

Orthopoxviruses are known to infect a variety of different mammals including rodents, domesticated animals, primates and humans. Several orthopoxviruses have a broad host range, while others have narrower host range. Exemplary orthopoxviruses include buffalopox, camelpox, cowpox, ectromelia, monkeypox, raccoon pox, skunk pox, tatera pox, uasin gishu, vaccinia, variola, and volepox viruses. In some embodiments, the orthopoxvirus selected can be an orthopoxvirus known to infect humans, such as cowpox, monkeypox, vaccinia, or variola virus. Optionally, the orthopoxvirus known to infect humans can be selected from the group of orthopoxviruses with a broad host range, such as cowpox, monkeypox, or vaccinia virus.

(1) Vaccinia Viruses

One exemplary orthopoxvirus for use in the methods provided herein is vaccinia virus. Vaccinia is a cytoplasmic virus, thus, it does not insert its genome into the host genome during its life cycle. The linear dsDNA viral genome of vaccinia virus is approximately 200 kb in size, encoding a total of approximately 200 potential genes. A variety of vaccinia virus strains are available for uses in the methods provided, including Western Reserve (WR), Copenhagen, Tashkent, Tian Tan, Lister, Wyeth, IHD-J, and IHD-W, Brighton, Ankara, MVA, Dairen I, LIPV, LC16M8, LC16MO, LIVP, WR 65-16, Connaught, New York City Board of Health. Exemplary vaccinia viruses are Lister or LIVP vaccinia viruses. In one embodiment, the Lister strain can be an attenuated Lister strain, such as the LIVP (Lister virus from the Institute of Viral Preparations, Moscow, Russia) strain, which was produced by further attenuation of the Lister strain. The LIVP strain was used for vaccination throughout the world, particularly in India and Russia, and is widely available. In another embodiment, the viruses and methods provided herein can be based on modifications to the Lister strain of vaccinia virus. Lister (also referred to as Elstree) vaccinia virus is available from any of a variety of sources. For example, the Elstree vaccinia virus is available at the ATCC under Accession Number VR-1549. The Lister vaccinia strain has high transduction efficiency in tumor cells with high levels of gene expression.

Vaccinia virus possesses a variety of features for use in cancer gene therapy and vaccination including broad host and cell type range, a large carrying capacity for foreign genes (up to 25 kb of exogenous DNA fragments (approximately 12% of the vaccinia genome size) can be inserted into the vaccinia genome), high sequence homology among different strains for designing and generating modified viruses in other strains, and techniques for production of modified vaccinia strains by genetic engineering are well established (Moss (1993) Curr. Opin. Genet. Dev. 3: 86-90; Broder and Earl (1999) Mol. Biotechnol. 13: 223-245; Timiryasova et al. (2001) Biotechniques 31: 534-540). A variety of vaccinia virus strains are available, including Western Reserve (WR), Copenhagen, Tashkent, Tian Tan, Lister, Wyeth, IHD-J, and IHD-W, Brighton, Ankara, MVA, Dairen I, LIPV, LC16M8, LC16MO, LIVP, WR 65-16, Connaught, New York City Board of Health. Exemplary of vaccinia viruses for use in the methods provided herein include, but are not limited to, Lister strain or LIVP strain of vaccinia viruses.

The exemplary modifications of the Lister strain described herein (see Example 1) also can be adapted to other vaccinia viruses (e.g., Western Reserve (WR), Copenhagen, Tashkent, Tian Tan, Lister, Wyeth, IHD-J, and IHD-W, Brighton, Ankara, MVA, Dairen I, LIPV, LC16M8, LC16MO, LIVP, WR 65-16, Connaught, New York City Board of Health). The modifications of the Lister strain described herein also can be adapted to other viruses, including, but not limited to, viruses of the poxvirus family, adenoviruses, herpes viruses and retroviruses.

(2) Modification of Vaccinia Viruses

Exemplary vaccinia viruses for use in the in methods provided include vaccinia viruses with insertions, mutations or deletions, as described elsewhere herein (see, e.g. Example 1). Exemplary insertions, mutations or deletions include those that result in an attenuated vaccinia virus relative to the wild type strain. For example, vaccinia virus insertions, mutations or deletions can decrease pathogenicity of the vaccinia virus, for example, by reducing the toxicity, reducing the infectivity, reducing the ability to replicate, or reducing the number of non-tumor organs or tissues to which the vaccinia virus can accumulate. Other exemplary insertions, mutations or deletions include, but are not limited to, those that increase antigenicity of the virus, those that permit detection, monitoring, or imaging, those that alter attenuation of the virus, and those that alter infectivity. For example, the ability of vaccinia viruses provided herein to infect and replicate within tumors can be enhanced by mutations that increase the extracellular enveloped form of the virus (EEV) that is released from the host cell, as described elsewhere herein. Modifications can be made, for example, in genes that are involved in nucleotide metabolism, host interactions and virus formation or at other nonessential gene loci. Any of a variety of insertions, mutations or deletions of the vaccinia virus known in the art can be used herein, including insertions, mutations or deletions of: the thymidine kinase (TK) gene, the hemagglutinin (HA) gene, and F14.5L gene, among others (e.g., E2L/E3L, K1L/K2L, superoxide dismutase locus, 7.5K, C7-K1L, J2R, B13R+B14R, A56R, A26L or I4L gene loci). The vaccinia viruses provided herein also can contain two or more insertions, mutations or deletions. Thus, included are vaccinia viruses containing two or more insertions, mutations or deletions of the loci provided herein or other loci known in the art. The viruses provided herein can be based on modifications to the Lister strain and/or LIVP strain of vaccinia virus. Any known vaccinia virus, or modifications thereof that correspond to those provided herein or known to those of skill in the art to reduce toxicity of a vaccinia virus. Generally, however, the mutation will be a multiple mutant and the virus will be further selected to reduce toxicity.

The modified viruses provided herein can contain one more heterologous nucleic acid sequences for the expression of a heterologous gene. The heterologous nucleic acid is typically operably linked to a promoter for expression of the heterologous gene in the infected cells. Suitable promoter include viral promoters, such as a vaccinia virus natural and synthetic promoters. Exemplary vaccinia viral promoters include, but are not limited to, P11k, P7.5k early/late, P7.5k early, P28 late, synthetic early P_{SE}, synthetic early/late P_{SEL} and synthetic late P_{SL} promoters.

The viruses provided herein can express one or more genes whose products are useful for tumor therapy. For example, a virus can express a proteins cause cell death or whose products cause an anti-tumor immune response. Such genes can be considered therapeutic genes. A variety of therapeutic gene products, such as toxic or apoptotic proteins, or siRNA, are known in the art, and can be used with the viruses provided herein. The therapeutic genes can act by directly killing the host cell, for example, as a channel-forming or other lytic protein, or by triggering apoptosis, or by inhibiting essential cellular processes, or by triggering an immune response against the cell, or by interacting with a compound that has a similar effect, for example, by converting a less active compound to a cytotoxic compound. Exemplary proteins useful for tumor therapy include, but are not limited to, tumor suppressors, toxins, cytostatic proteins, antiangiogenic proteins, antitumor antibodies, and costimulatory molecules, such as cytokines and chemokines among others provided elsewhere herein and known in the art. The viruses provided herein can also be effective against tumors without the introduction of additional exogenous therapeutic genes.

The viruses provided herein can express one or more genes whose products are useful for tumor detection and/or imaging. Exemplary gene products for imaging or detection include detectable proteins or proteins that induce detectable signals. Exemplary of detectable proteins or proteins that induce detectable signals are proteins, such as luciferases, fluorescent proteins, receptors that can bind imaging agents, or proteins linked to imaging or diagnostic moieties. The viruses provided herein also can encode proteins, such as transporter proteins (e.g., the human norepinephrine transporter (hNET) or the human sodium iodide symporter (hNIS)), which can provide increase uptake diagnostic and therapeutic moieties across the cell membrane of infected cells for therapy, imaging or detection.

Imaging or diagnostic moieties include those that can emit a signal that is detectable by optical or non-optical imaging methods. Detection of the signal by imaging modalities such as, for example, by positron emission tomography (PET) and, thereby allows visualization of the infected tissues, such a tumor or an inflammation.

One skilled in the art can select from any of a variety of viruses, according to a variety of factors, including, but not limited to, the intended use of the virus, such as a diagnostic and/or therapeutic use (e.g., tumor therapy or diagnosis, vaccination, antibody production, or heterologous protein production), the host organism, and the type of tumor. An oncolytic virus for the methods provided herein can exhibit one or more desired characteristics for use as a therapeutic agent, such as, for example attenuated pathogenicity, reduced toxicity, preferential accumulation in immunoprivileged cells and tissues, such as tumor, ability to activate an immune response against tumor cells, immunogenic, replication competent, and are able to express exogenous proteins, and combinations thereof.

(3) Exemplary Modified Vaccinia Viruses

Exemplary vaccinia viruses contemplated for use in the methods provided include those derived from vaccinia virus strain GLV-1h68 (also named RVGL21, SEQ ID NO: 1), which has been described in U.S. Pat. Pub. No. 2005-0031643 and is incorporated herein by reference in its entirety. GLV-1h68 contains DNA insertions gene loci of the vaccinia virus LIVP strain (SEQ ID NO: 2, a vaccinia virus strain, originally derived by adapting the Lister strain (ATCC Catalog No. VR-1549) to calf skin (Institute of Viral Preparations, Moscow, Russia, Al'tshtein et al., (1983) Dokl. Akad. Nauk USSR 285:696-699)). GLV-1h68 contains expression cassettes encoding detectable marker proteins in the F14.5L (also designated in LIVP as F3), thymidine kinase (TK) and hemagglutinin (HA) gene loci. An expression cassette containing a Ruc-GFP cDNA molecule (a fusion of DNA encoding Renilla luciferase and DNA encoding GFP) under the control of a vaccinia synthetic early/late promoter P_{SEL} ((P_{SEL})Ruc-GFP) is inserted into the F14.5L gene locus; an expression cassette containing a DNA molecule encoding beta-galactosidase under the control of the vaccinia early/late promoter P_{7.5k} ((P_{7.5k})LacZ) and DNA encoding a rat transferrin receptor positioned in the reverse orientation for transcription relative to the vaccinia synthetic early/late promoter P_{SEL} ((P_{SEL})rTrfR) is inserted into the TK gene locus (the resulting virus does not express transferrin receptor protein since the DNA molecule encoding the protein is positioned in the reverse orientation for transcription relative to the promoter in the cassette); and an expression cassette containing a DNA molecule encoding β-glucuronidase under the control of the vaccinia late promoter P₁₁ₖ ((P₁₁ₖ)gusA) is inserted into the HA gene locus. The GLV-1h68 virus exhibits a strong preference for accumulation in tumor tissues as compared to non-tumorous tissues following systemic administration of the virus to tumor bearing subjects. This preference is significantly higher than the tumor selective accumulation of other vaccinia viral strains, such as WR (see, e.g. U.S. Pat. Pub. No. 2005-0031643 and Zhang et al. (2007) Cancer Res. 67(20):10038-46). Modified viruses provided herein for the uses and methods provided can be derived from GLV-1h68. Exemplary viruses are generated by replacement of one or more expression cassettes of the GLV-1h68 strain with heterologous DNA encoding gene products for therapy and/or imaging.

Non-limiting examples viruses that are derived from attentuated LIVP viruses, such as GLV-1h68, and can be employed in the methods and uses provided include, but are not limited to, LIVP viruses described in U.S. Patent Publication Nos. 2005/0031643, 2004/0234455 and 2004/0213741 and U.S. patent application Ser. Nos. 11/975,088, 11/975,090, and 12/157,960, which are incorporated herein by reference in their entirety. For example, the vaccinia virus can be selected from among GLV-1h22, GLV-1h68, GLV-1i69, GLV-1h70, GLV-1h71, GLV-1h72, GLV-1h73, GLV-1h74, GLV-1h81, GLV-1h82, GLV-1h83, GLV-1h84, GLV-1h85, or GLV-1h86, which are described in U.S. application Ser. No. 11/975,088 and GLV-1h104, GLV-1h105, GLV-1h106, GLV-1h107, GLV-1h108 and GLV-1h109, which are described in U.S. application Ser. No. 11/975,090; GLV-1h99, GLV-1h100, GLV-1h101, GLV-1h139, GLV-1h146, GLV-1h151, GLV-1h152 and GLV-1h153, which are described in U.S. application Ser. No. 12/157,960. A description of each of these strains is provided herein (e.g., see Example 1).

Exemplary of viruses which have one or more expression cassettes removed from GLV-1h68 and replaced with a heterologous non-coding DNA molecule include GLV-1h70, GLV-1h71, GLV-1h72, GLV-1h73, GLV-1h74, GLV-1h85, and GLV-1h86. GLV-1h70 contains (P_{SEL})Ruc-GFP inserted into the F14.5L gene locus, (P_{SEL})rTrfR and (P_{7.5k})LacZ inserted into the TK gene locus, and a non-coding DNA molecule inserted into the HA gene locus in place of (P₁₁ₖ)gusA. GLV-1h71 contains a non-coding DNA molecule inserted into the F14.5L gene locus in place of (P_{SEL})Ruc-GFP, (P_{SEL})rTrfR and (P_{7.5k})LacZ inserted into the TK gene locus, and (P₁₁ₖ)gusA inserted into the HA gene locus. GLV-1h72 contains (P_{SEL})Ruc-GFP inserted into the F14.5L gene locus, a non-coding DNA molecule inserted into the TK gene locus in place of (P_{SEL})rTrfR and (P_{7.5k})LacZ, and P₁₁ₖgusA inserted into the HA gene locus. GLV-1h73 contains a non-coding DNA molecule inserted into the F14.5L gene locus in place of (P_{SEL})Ruc-GFP, (P_{SEL})TrfR and (P_{7.5k})LacZ inserted into the TK gene locus, and a non-coding DNA molecule inserted into the HA gene locus in place of (P₁₁ₖ)gusA. GLV-1h74 contains a non-coding DNA molecule inserted into the F14.5L gene locus in place of (P_{SEL})Ruc-GFP, a non-coding DNA molecule inserted into the TK gene locus in place of (P_{SEL})rTrfR and (P_{7.5k})LacZ, and a non-coding DNA molecule inserted into the HA gene locus in place of (P₁₁ₖ)gusA. GLV-1h85 contains a non-coding DNA molecule inserted into the F14.5L gene locus in place of (P_{SEL})Ruc-GFP, a non-coding DNA molecule inserted into the TK gene locus in place of (P_{SEL})rTrfR and (P_{7.5k})LacZ, and (P₁₁ₖ)gusA inserted into the HA gene locus. GLV-1h86 contains (P_{SEL})Ruc-GFP inserted into the F14.5L gene locus, a non-coding DNA molecule inserted into the TK gene locus in place of (P_{SEL})rTrfR and (P_{7.5k})LacZ, and a non-coding DNA molecule inserted into the HA gene locus in place of (P₁₁ₖ)gusA.

Other exemplary viruses include, but are not limited to, LIVP viruses that express one or more therapeutic gene products, such as angiogenesis inhibitors (e.g., GLV-1h81, which contains DNA encoding the plasminogen K5 domain (SEQ ID NO: 43) under the control of the vaccinia synthetic early-late promoter in place of the gusA expression cassette at the HA locus in GLV-1h68; GLV-1h104, GLV-1h105 and GLV-1h106, which contain DNA encoding a truncated human tissue factor fused to the αᵥβ₃-integrin RGD binding motif (tTF-RGD) (SEQ ID NO: 93) under the control of a vaccinia synthetic early promoter, vaccinia synthetic early/late promoter or vaccinia synthetic late promoter, respectively, in place of the LacZ/rTFr expression cassette at the TK locus of GLV-1h68; GLV-1h107, GLV-1h108 and GLV-1h109, which contain DNA encoding an anti-VEGF single chain antibody G6 (SEQ ID NO: 99) under the control of a vaccinia synthetic early promoter, vaccinia synthetic early/late promoter or vaccinia synthetic late promoter, respectively, in place of the LacZ/rTFr expression cassette at the TK locus of GLV-1h68) and proteins for tumor growth suppression (e.g., GLV-1h90, GLV-1h91 and GLV-1h92, which express a fusion protein containing an IL-6 fused to an IL-6 receptor (sIL-6R/IL-6) (SEQ ID NO: 106) under the control of a vaccinia synthetic early promoter, vaccinia synthetic early/late promoter or vaccinia synthetic late promoter, respectively, in place of the gusA expression cassette at the HA locus in GLV-1h68; and GLV-1h96, GLV-1h97 and GLV-1h98, which express IL-24 (melanoma differentiation gene, mda-7; SEQ ID NO: 107) under the control of a vaccinia synthetic early promoter, vaccinia synthetic early/late promoter or vaccinia synthetic late promoter, respectively, in place of the Ruc-GFP fusion gene expression cassette at the F14.5L locus of GLV-1h68). Additional therapeutic gene products that can be engineered in the viruses provided herein also are described elsewhere herein.

Exemplary transporter proteins that can be encoded by the viruses provided herein include, for example, the human norepinephrine transporter (hNET; SEQ ID NO: 142 (cDNA), 141 (protein)) and the human sodium iodide symporter (hNIS; SEQ ID NO: 143 (cDNA), 142 (protein)). Exemplary viruses that can be employed in the methods and use provided herein that encode the human norepinephrine transporter (hNET) include, but are not limited to, GLV-1h99, GLV-1h100, GLV-1h101, GLV-1h139, GLV-1h146, GLV-1h150, GLV-1h151 and GLV-1h152. GLV-1h99 encodes hNET under the control of a vaccinia synthetic early promoter in place of the Ruc-GFP fusion gene expression cassette at the F14.5L locus of GLV-1h68. GLV-1h100, GLV-1h101 encode hNET under the control of a vaccinia synthetic early promoter or vaccinia synthetic late promoter, respectively, in place of the LacZ/rTFr expression cassette at the TK locus of GLV-1h68. GLV-1h39 encodes hNET under the control of a vaccinia synthetic early promoter in place of the gusA expression cassette at the HA locus in GLV-1h68. GLV-1h146 and GLV-1h150, encode hNET under the control of a vaccinia synthetic early promoter or vaccinia synthetic late promoter, respectively, in place of the LacZ/rTFr expression cassette at the TK locus of GLV-1h100 and GLV-101, respectively. Thus, GLV-1h146 and GLV-1h150 encode both hNET and IL-24. Exemplary viruses that can be employed in the methods and use provided herein that encode the human sodium iodide transporter (hNIS) include, but are not limited to, GLV-1h151, GLV-1h151 and GLV-1h153. GLV-1h151, GLV-1h151 and GLV-1h153 encode hNIS under the control of a vaccinia synthetic early promoter, vaccinia synthetic early/late promoter or vaccinia synthetic late promoter, respectively, in place of the gusA expression cassette at the HA locus in GLV-1h68.

Other exemplary viruses include, but are not limited to, LIVP viruses that encode additional imaging agents such as ferritin and/or a transferrin receptor (e.g., GLV-1h82 and GLV-1h83 which encode E. coli ferritin at the HA locus; GLV-1h82 addition encodes the human transferrin receptor at the TK locus) or a click beetle luciferase-red fluorescent protein fusion protein (e.g., GLV-1h84, which encodes CBG99 and mRFP1 at the TK locus). During translation, the two proteins are cleaved into two individual proteins at picornavirus 2A element (Osborn et al., Mol. Ther. 12: 569-74, 2005). CBG99 produces a more stable luminescent signal than does Renilla luciferase with a half-life of greater than 30 minutes, which makes both in vitro and in vivo assays more convenient. mRFP1 provides improvements in in vivo imaging relative to GFP since mRFP1 can penetrate tissue deeper than GFP.

b. Other Cytoplasmic Viruses

Other viruses that can be used in the methods provided herein include cytoplasmic viruses that are not poxviruses. A variety of such cytoplasmic viruses are known in the art, and include African swine flu family viruses and various RNA viruses such as arenaviruses, picornaviruses, caliciviruses, togaviruses, coronaviruses, paramyxoviruses, flaviviruses, reoviruses, and rhaboviruses. Exemplary togaviruses include Sindbis viruses. Exemplary arenaviruses include lymphocytic choriomeningitis virus. Exemplary rhaboviruses include vesicular stomatitis viruses. Exemplary paramyxoviruses include Newcastle Disease viruses and measles viruses. Exemplary picornaviruses include polio viruses, bovine enteroviruses and rhinoviruses. Exemplary flaviviruses include Yellow fever virus; attenuated Yellow fever viruses are known in the art, as exemplified in Barrett et al. (Biologicals 25: 17-25 (1997)), and McAllister et al. (J. Virol. 74: 9197-9205 (2000)).

Also provided herein are modifications of the viruses provided above to enhance one or more characteristics relative to the wild type virus. Such characteristics can include, but are not limited to, attenuated pathogenicity, reduced toxicity, preferential accumulation in tumor, increased ability to activate an immune response against tumor cells, increased immunogenicity, increased or decreased replication competence, and are able to express exogenous proteins, and combinations thereof. In some embodiments, the modified viruses have an ability to activate an immune response against tumor cells without aggressively killing the tumor cells. In other embodiments, the viruses can be modified to express one or more detectable genes, including genes that can be used for imaging. In other embodiments, the viruses can be modified to express one or more genes for harvesting the gene products and/or for harvesting antibodies against the gene products.

c. Adenovirus, Herpes, Retroviruses

Other viruses that can be used in the methods provided herein include viruses that include in their life cycle entry of a nucleic acid molecule into the nucleus of the host cell. A variety of such viruses is known in the art, and includes herpesviruses, papovaviruses, retroviruses, adenoviruses, parvoviruses and orthomyxoviruses. Exemplary herpesviruses include herpes simplex type 1 viruses, cytomegaloviruses, and Epstein-Barr viruses. Exemplary papovaviruses include human papillomavirus and SV40 viruses. Exemplary retroviruses include lentiviruses. Exemplary orthomyxoviruses include influenza viruses. Exemplary parvoviruses include adeno associated viruses.

2. Exemplary Stem Cell Compositions

Exemplary stem cell compositions for use in the methods herein include, but are not limited to, compositions containing embryonic stem cells (ESCs), germinal stem cells, and/or adult stem cells (ASCs). ASCs include, but are not limited to, hematopoietic stems cells (HSCs) from bone marrow, and other primitive progenitor cells such as mesenchymal stem cells (MSCs) and multipotent adult progenitor cells (PAPCs). The stem cells in the stem cell compositions provided herein can be of human origin or non-human origin, including, but not limited to, mouse, rat or non-human primate origin. In one example, the stem cell compositions contain ESCs. For example, exemplary stem cell compositions for use in the methods herein include, but are not limited to, those containing human embryonic stem cells, such as any set forth in Table 1. For example, the stem cell compositions can contain embryonic stem cells selected from among ACT-14, AS034, AS034.1, AS034.2, AS038, AS079, AS094, BG01, BG02, BG03, BG04, CH01, CH02, CLS1, CLS2, CLS3, CLS4, ES01, ES02, ES03, ES04, ES05, ES06, ESM01, ESM02, ESM03, FC018, FES 21, FES 22, FES 29, FES 30, GE01, GE07, GE09, GE13, GE14, GE91, GE92, hES-NCL1, HS181, HS207, HUES1, HUES10, HUES11, HUES12, HUES13, HUES14, HUES15, HUES16, HUES17, HUES2, HUES3, HUES4, HUES5, HUES6, HUES7, HUES8, HUES9, KhES-1, KhES-2, KhES-3, MB01, MB02, MB03, Miz-hES1, Miz-hES10, Miz-hES11, Miz-hES12, Miz-hES13, Miz-hES14, Miz-hES15, Miz-hES2, Miz-hES3, Miz-hES4, Miz-hES5, Miz-hES6, Miz-hES7, Miz-hES8, NC01, NC02, NC03, ReliCellhES1, RH1, RH3, RH4, RH5, RH6, RH7, RL05, RL07, RL10, RL13, RL15, RL20, RL21, Royan H1, SA001, SA002, SA002.5, SA046, SA085, SA111, SA121, SA142, SA167, SA181, SA191, SA196, SA202, SA203, SA211, SA218, SA240, SA279, SA348, SA352, SA399, SA611, SI-100, SI-101, SI-102, SI-103, SI-104, SI-105, SI-106, SI-107, SI-108, SI-109, SI-110, SI-111, SI-114, SI-115, SI-122, SI-123, SI-124, SI-125, SI-126, SI-128, SI-130, SI-131, SI-132, SI-133, SI-134, SI-135, SI-137, SI-138, SI-139, SI-140, SI-141, SI-144, SI-145, SI-146, SI-148, SI-149, SI-15, SI-150, SI-151, SI-153, SI-154, SI-155, SI-156, SI-157, SI-158, SI-159, SI-160, SI-161, SI-162, SI-163, SI-164, SI-165, SI-167, SI-168, SI-169, SI-170, SI-171, SI-172, SI-174, SI-175, SI-176, SI-177, SI-178, SI-179, SI-18, SI-180, SI-182, SI-183, SI-184, SI-185, SI-186, SI-187, SI-188, SI-189, SI-191, SI-192, SI-193, SI-194, SI-195, SI-196, SI-197, SI-198, SI-199, SI-200, SI-201, SI-202, SI-203, SI-204, SI-205, SI-206, SI-208, SI-209, SI-21, SI-210, SI-211, SI-213, SI-214, SI-215, SI-216, SI-217, SI-221, SI-24, SI-27, SI-28, SI-31, SI-33, SI-53, SI-60, SI-62, SI-63, SI-79, SI-80, SI-81, SI-93, SI-94, SI-95, SI-96, SI-97, SI-98, SI-99, SNUhES1, SNUhES2, SNUhES3, TE-03, TE-04, TE-06, TE-07, TE-32, TE-33, TE-62, TE-72, UC01, UC06, VAL-1, VAL-2, VAL-3, VAL-4, WA01, WA07, WA09, WA13 or WA14 cells.

The stem cell compositions can contain culture adapted stem cells or nonadapted stem cells, and can contain one or more type of stem cell. For example, included among the stem cell compositions for use in the methods are those that contain ESCs from two or more ESC lines, or those that contain ESCs and ASCs. In other examples, the stem cells can be partially differentiated (e.g., derivative stem cells). In some examples, the stem cells of the stem cell composition can be partially differentiated in vitro prior to administration to a subject or prior to contact with the virus, according to known methods in the art for differentiating stem cells. For example, the stem cells can be differentiated for have been differentiated in vitro for 1, 2, 3, 4, 5, 6, 7, 8, 9, 10, 11, 12, 13, 14, 15, 16, 17, 18, 19, 20, 25 or 30 days. The stem cell compositions ca

3. Methods of Treatment

a. Administration of Virus for Prevention of Tumor Formation

i. Direct Administration of Virus

Provided are methods of administering a stem cell composition in combination with an oncolytic virus to a subject in need of stem cell therapy. In such methods, administration of the oncolytic virus inhibits tumor formation resulting from the stem cell therapy. In exemplary methods, the virus is administered directly to a subject that is administered a stem cell composition for the treatment of a disease or disorder. The administered virus accumulates in and infects that administered stem cell composition in vivo and can eliminate neoplastic cells or neoplastic progenitor cells of the administered stem cell composition in vivo. The oncolytic virus provided herein can be administered concurrently, sequentially, or intermittently with the stem cell composition. For example, the virus can be administered to the subject following the administration of the stem cell composition, such as, for example, about or 5 minutes, 10 minutes, 15 minutes, 30 minutes, 1 hour, 2 hours, 3 hours, 4 hours, 5 hours, 6 hours, 12 hours, 24 hours, 30 hours, 36 hours, 42 hours, 48 hours, 3 days, 4 days, 5 days, 6 days, 7 days, 8 days, 9 days, 10 days, 11 days, 12 days, 13 days, 14 days, 2 weeks, 3 weeks, 4 weeks, 5 week, 6 weeks or more following the administration of the stem cell composition. The stem cell composition and/or the virus also can be administered multiple times as described herein.

The virus and the stem cell composition can be administered in the same composition or in separate compositions. The virus and the stem cell composition can be administered via the same route of administration or different routes of administration as provided herein. For example, the stem cell composition and the virus can be administered locally or systemically, together in the same composition or in separate compositions, via the same route of administration or different routes of administration.

(1) Mode of Administration

Any mode of administration of a stem cell composition can be employed provided the mode of administration permits the stem cell composition to treat the disease or disorder in the subject for which stem cell therapy is administered. For example, the stem cell composition can be administered intraarterially, intratumorally, endoscopically, intralesionally, intramuscularly, intradermally, intraperitoneally, intravesicularly, intraarticularly, intrapleurally, percutaneously, subcutaneously, orally, parenterally, intranasally, intratracheally, by inhalation, intracranially, intraprostaticaly, intravitreally, ocularly, vaginally, intracoronary, intramyocardially, transendocardially, trans-epicardially, intraspinally, intra-striatumly, transdermally, rectally or sub-epidermally. In some examples, the stem cell composition can be administered locally, by implantation of the stem cell composition at the site for therapy (e.g. at the site of tissue or cell damage). In other examples, the stem cell composition can be administered systemically, such as, for example, by intravenous or parenteral administration. Modes of administration of stem cells are known in the art. One skilled in the art can select any mode of administration compatible with the subject and the disease or disorder to be treated.

In some exemplary methods, a laminectomy can be performed under general anesthetic to administer the stem cell composition for the treatment of disorders, such as spinal cord injury. In such methods, the injured vertebra and the dura are surgically exposed, and the stem cell composition is injected directly into the damaged tissue. In other exemplary methods, the stem cell compositions can be administered directly into the brain via catheter for treatment of brain injuries such as, for example, stroke. In other exemplary methods, the stem cell composition can be administered by lumbar puncture into the cerebrospinal fluid in patients with neurological diseases such as Alzheimer's or multiple sclerosis. In other exemplary methods, the stem cell composition can be administered by angiography to target organs, such as the liver, heart or pancreas. In such methods, a catheter is inserted into the femoral artery under local anesthetic and guided to the target organ for delivery of the stem cell compositions. Such methods can be employed, for example, for the treatment of diabetes mellitus in order to deliver the stem cells directly to the pancreas, or for patients who have had a cardiac arrest or suffer from cardiac insufficiency (weak heart).

Any mode of administration of an oncolytic virus to a subject can be used, provided the mode of administration permits the virus to infect neoplastic cells of the administered stem cell composition or tumor cells of the stem cell-derived tumor. Modes of administration can include, but are not limited to, systemic, intravenous, intraperitoneal, subcutaneous, intramuscular, transdermal, intradermal, intra-arterial (e.g., hepatic artery infusion), intravesicular perfusion, intrapleural, intraarticular, topical, intratumoral, intralesional, multipuncture (e.g., as used with smallpox vaccines), inhalation, percutaneous, subcutaneous, intranasal, intratracheal, oral, intracavity (e.g., administering to the bladder via a catheter, administering to the gut by suppository or enema), vaginal, rectal, intracranial, intraprostatic, intravitreal, aural, or ocular administration. One skilled in the art can select any mode of administration compatible with the subject and the virus, and that also is likely to result in the virus reaching stem cell implantation site or stem cell-derived tumor.

ii Pre-Treatment of Stem Cells for Administration

Provided are methods of administering a stem cell composition that has been pre-treated with an oncolytic virus for the treatment of a disease or disorder in a subject for which stem cell therapy is administered. In such methods, the stem cell composition to be administered is first contacted with the virusto permit infection of neoplastic cells or neoplastic progenitor cells in the stem cell composition. The virus can preferentially infect and replicate in neoplastic cells and neoplastic progenitor cells and eliminate such cells from the stem cell composition. The virus is added to the stem cell composition at a an appropriate multiplicity of infection (MOI). An appropriate MOI can be selected based on the cell type infected and the virus. Typical MOIs include, but are not limited to, 0.001, 0.01, 0.1, 1, 10 and 100. At a selected time point following infection, the pre-treated stem cell composition is administered to a subject for the treatment of a disease or disorder to be treated by stem cell therapy. The infection time selected should be sufficient to permit infection of the stem cell composition. In some examples, the stem cell composition is contacted with the virus for 1, 2, 3, 4, 5, 6, 7, 8, 9, 10, 11, 12, 18, 24, 30, 36, 42, 48, or 72 hours or more prior to administration of the pretreated stem cell composition.

Any mode of administration of a stem cell composition can be employed to administer a pre-treated stem cell composition provided the mode of administration permits the stem cell composition to treat the disease or disorder. Exemplary modes of administration of a stem composition are provided elsewhere herein.

iii. Administration of Virus for Treatment of a Stem Cell-Derived Tumor

Provided are methods of administering an oncolytic virus for the treatment of a subject having a tumor resulting from stem cell therapy. In such methods, a subject selected for administration of the virus is one who has been diagnosed as having a tumor following administration of a stem cell therapy. Such tumors are typically are derived from the stem cell compositions that are administered for therapy. In some examples, as described herein, a virus, such as the viruses described herein, can be employed for detection and imaging of the stem cell-derived tumor in a subject suspected of having a tumor and also can be employed for the treatment of the tumor. In addition, as described herein, such viruses can be employed for the monitoring of treatment of the tumor.

Any mode of administration of an oncolytic virus to a subject can be used, provided the mode of administration permits the virus to infect tumor cells of the stem cell-derived tumor. Exemplary modes of administration of a virus to a subject are provided elsewhere herein.

b. Virus Dosages

The dosage regimen for administering an oncolytic virus can be any of a variety of methods and amounts, and can be determined by one skilled in the art according to known clinical factors. As is known in the medical arts, dosages for any one patient can depend on many factors, including the subject's species, size, body surface area, age, sex, immunocompetence, and general health, the particular virus to be administered, duration and route of administration, the kind and stage of the disease, for example, tumor size, and other treatments or compounds, such as chemotherapeutic drugs, being administered concurrently. In addition to the above factors, such levels can be affected by the infectivity of the virus, and the nature of the virus, as can be determined by one skilled in the art. In the present methods, appropriate minimum dosage levels of viruses can be levels sufficient for the virus to survive, grow and replicate in a neoplastic cells or neoplastic progenitor cells of a stem-cell composition or stem-cell derived tumor. Exemplary minimum levels for administering a virus to a 65 kg human can include at least or about 1×10⁵ plaque forming units (PFU), at least or about 5×10⁵ PFU, at least or about 1×10⁶ PFU, at least or about 5×10⁶ PFU, at least or about 1×10⁷ PFU, at least or about 5×10⁷ PFU, at least or about 1×10⁸ PFU, at least or about 5×10⁸ PFU, at least or about 1×10⁹ PFU, at least or about 5×10⁹ PFU, at least or about 1×10¹⁰ PFU or at least or about 5×10¹⁰ PFU. In the present methods, appropriate maximum dosage levels of viruses can be levels that are not toxic to the host, levels that do not cause splenomegaly of 3 times or more, levels that do not result in colonies or plaques in normal tissues or organs after about 1 day or after about 3 days or after about 7 days. Exemplary maximum levels for administering a virus to a 65 kg human can include no more than about 1×10¹¹ PFU, no more than about 5×10¹⁰ PFU, no more than about 1×10¹⁰ PFU, no more than about 5×10⁹ PFU, no more than about 1×10⁹ PFU, or no more than about 1×10⁸ PFU.

c. Number of Administrations

The methods provided herein can include a single administration of a virus to a subject or multiple administrations of a virus to a subject. In some embodiments, a single administration is sufficient to establish a virus in a stem cell implant or stem cell-derived tumor, where the virus can proliferate and can cause or enhance an anti-neoplastic or an anti-tumor response in the subject; such methods do not require additional administrations of a virus in order to cause or enhance an anti-neoplastic or an anti-tumor response in a subject, which can result, for example in elimination of neoplastic cells from the implanted stem cell composition, inhibition of growth of a stem cell-derived tumor, inhibition of metastasis growth or formation from a stem cell-derived tumor, reduction in tumor or size, elimination of a tumor or metastasis, inhibition or prevention of recurrence of a neoplastic disease or new tumor formation, or other cancer therapeutic effects.

In other examples, a virus can be administered on different occasions, separated in time typically by at least one day. Separate administrations can increase the likelihood of delivering a virus to a stem cell implant or stem cell derived-tumor, where a previous administration has been ineffective in delivering a virus to the stem cell implant or stem cell derived-tumor. Separate administrations can increase the locations on stem cell derived-tumor where virus proliferation can occur or can otherwise increase the titer of virus accumulated in the stem cell implant or stem cell derived-tumor, which can increase the scale of release of antigens or other compounds from the tumor cells in eliciting or enhancing a host's anti-tumor immune response, and also can, optionally, increase the level of virus-based tumor lysis or tumor cell death. Separate administrations of a virus can further extend a subject's immune response against viral antigens, which can extend the host's immune response to tumors or metastases in which viruses have accumulated, and can increase the likelihood of a host mounting an anti-neoplastic or anti-tumor immune response.

When separate administrations are performed, each administration can be a dosage amount that is the same or different relative to other administration dosage amounts. In one embodiment, all administration dosage amounts are the same. In other embodiments, a first dosage amount can be a larger dosage amount than one or more subsequent dosage amounts, for example, at least 10× larger, at least 100× larger, or at least 1000× larger than subsequent dosage amounts. In one example of a method of separate administrations in which the first dosage amount is greater than one or more subsequent dosage amounts, all subsequent dosage amounts can be the same, smaller amount relative to the first administration.

Separate administrations can include any number of two or more administrations, including two, three, four, five or six administrations. One skilled in the art can readily determine the number of administrations to perform or the desirability of performing one or more additional administrations according to methods known in the art for monitoring therapeutic methods and other monitoring methods provided herein. Accordingly, the methods provided herein include methods of providing to the subject one or more administrations of a virus, where the number of administrations can be determined by monitoring the subject, and, based on the results of the monitoring, determining whether or not to provide one or more additional administrations. Deciding on whether or not to provide one or more additional administrations can be based on a variety of monitoring results, including, but not limited to, indication of elimination or neoplastic cells in the stem cell composition, tumor growth or inhibition of tumor growth, appearance of new metastases or inhibition of metastasis, the subject's anti-virus antibody titer, the subject's anti-tumor antibody titer, the overall health of the subject, the weight of the subject, the presence of virus solely in tumor and/or metastases, the presence of virus in normal tissues or organs.

The time period between administrations can be any of a variety of time periods. The time period between administrations can be a function of any of a variety of factors, including monitoring steps, as described in relation to the number of administrations, the time period for a subject to mount an immune response, the time period for a subject to clear the virus from normal tissue, or the time period for virus proliferation in the tumor or metastasis. In one example, the time period can be a function of the time period for a subject to mount an immune response; for example, the time period can be more than the time period for a subject to mount an immune response, such as more than about one week, more than about ten days, more than about two weeks, or more than about a month; in another example, the time period can be less than the time period for a subject to mount an immune response, such as less than about one week, less than about ten days, less than about two weeks, or less than about a month. In another example, the time period can be a function of the time period for a subject to clear the virus from normal tissue; for example, the time period can be more than the time period for a subject to clear the virus from normal tissue, such as more than about a day, more than about two days, more than about three days, more than about five days, or more than about a week. In another example, the time period can be a function of the time period for virus proliferation in the tumor or metastasis; for example, the time period can be more than the amount of time for a detectable signal to arise in a tumor or metastasis after administration of a virus expressing a detectable marker, such as about 3 days, about 5 days, about a week, about ten days, about two weeks, or about a month.

d. Steps Prior to Administration of the Virus

In some examples, one or more steps can be performed prior to administration of the virus to the subject. Any of a variety of preceding steps can be performed depending on whether the virus is administered for the prevention of a stem cell-derived tumor or is administered for the treatment of a patient having a stem cell-derived tumor, including, but not limited to diagnosing the subject with a condition appropriate for virus administration, determining the immunocompetence of the subject, immunizing the subject, treating the subject with a chemotherapeutic agent, treating the subject with radiation, or surgically treating the subject. Diagnostic methods can include determining the type of neoplastic condition, determining the stage of the neoplastic condition, determining the size of one or more tumors in the subject, determining the presence or absence of metastatic or neoplastic cells in the lymph nodes of the subject, or determining the presence of metastases of the subject. In some examples, prior to administering to the subject a virus, the immunocompetence of the subject can be determined to ensure that the subject is able to clear the virus from non-tumorous tissues or can mount an immune response against a tumor. The methods of administering a virus to a subject provided herein can include causing or enhancing an immune response in a subject. Accordingly, prior to administering a virus to a subject, the ability of a subject to mount an immune response can be determined. Any of a variety of tests of immunocompetence known in the art can be performed in the methods provided herein. Exemplary immunocompetence tests can examine ABO hemagglutination titers (IgM), leukocyte adhesion deficiency (LAD), granulocyte function (NBT), T and B cell quantitation, tetanus antibody titers, salivary IgA, skin test, tonsil test, complement C3 levels, and factor B levels, and lymphocyte count. One skilled in the art can determine the desirability to administer a virus to a subject according to the level of immunocompetence of the subject, according to the immunogenicity of the virus, and, optionally, according to the immunogenicity of the neoplastic disease to be treated. Typically, a subject can be considered immunocompetent if the skilled artisan can determine that the subject is sufficiently competent to mount an immune response against the virus.

In some examples, the subject can be immunized prior to administering to the subject a virus according to the methods provided herein. Immunization can serve to increase the ability of a subject to mount an immune response against the virus, or increase the speed at which the subject can mount an immune response against a virus. Immunization also can serve to decrease the risk to the subject of pathogenicity of the virus. In some embodiments, the immunization can be performed with an immunization virus that is similar to the oncolytic virus to be administered. For example, the immunization virus can be a replication-incompetent variant of the oncolytic virus. In other embodiments, the immunization material can be digests of the oncolytic virus to be administered. Any of a variety of methods for immunizing a subject against a known virus are known in the art and can be used herein. In one example, vaccinia viruses treated with, for example, 1 microgram of psoralen and ultraviolet light at 365 nm for 4 minutes, can be rendered replication incompetent. In another embodiment, the virus can be selected as the same or similar to a virus against which the subject has been previously immunized, e.g., in a childhood vaccination.

In some examples, prior to administration of a virus to a subject having a stem cell-derived tumor, the subject can be treated in one or more cancer treatment steps, including but not limited to administering a compound that decreases the rate of proliferation of the tumor or neoplastic cells without weakening the immune system (e.g., by administering tumor suppressor compounds or by administering tumor cell-specific compounds) or administering an angiogenesis-inhibiting compound. Thus, combined methods that include administering a virus to a subject can further improve cancer therapy. Thus, provided herein are methods of administering a virus to a subject, along with prior to or subsequent to, for example, administering a compound that slows tumor growth without weakening the subject's immune system or a compound that inhibits vascularization of the tumor.

4. Co-Administrations

Provided are methods for the treatment of a stem cell-derived tumor in which an additional therapeutic substance, such as a different oncolytic virus or a therapeutic compound is administered. These can be administered simultaneously, sequentially or intermittently with the first virus. The additional therapeutic substance can interact with the virus or a gene product thereof, or the additional therapeutic substance can act independently of the virus.

Combination therapy treatment has advantages in that: 1) it avoids single agent resistance; 2) in a heterogeneous tumor population, it can kill cells by different mechanisms; and 3) by selecting drugs with non-overlapping toxicities, each agent can be used at full dose to elicit maximal efficacy and synergistic effect. Combination therapy can be done by combining a diagnostic/oncolytic virus with one or more of the following anti-cancer agents: chemotherapeutic agents, therapeutic antibodies, siRNAs, toxins, enzyme-prodrug pairs or radiation.

a. Administering a Plurality of Viruses

Methods are provided for administering to a subject two or more viruses. Administration can be effected simultaneously, sequentially or intermittently. The plurality of viruses can be administered as a single composition or as two or more compositions. The two or more viruses can include at least two viruses. In some examples, where there are two viruses, both viruses are vaccinia viruses. In another example, one virus is a vaccinia virus and the second viruses is any one of an adenovirus, an adeno-associated virus, a retrovirus, a herpes simplex virus, a reovirus, a mumps virus, a foamy virus, an influenza virus, a myxoma virus, a vesicular stomatitis virus, or any other virus described herein or known in the art. Viruses can be chosen based on the pathway on which they act. For example, a virus that targets an activated Ras pathway can be combined with a virus that targets tumor cells defective in p53 expression.

The plurality of viruses can be provided as combinations of compositions and/or as kits that include the viruses packaged for administration and optionally including instructions therefore. Such combinations also can include stem cell compositions for therapy. The compositions can contain the viruses formulated for single dosage administration (i.e., for direct administration) or multiple dosage formulation (e.g., for multiple direct administrations), and can require dilution or other additions.

The viruses can be administered at approximately the same time, or can be administered at different times. The viruses can be administered in the same composition or in the same administration method, or can be administered in separate composition or by different administration methods. The time period between administrations can be any time period that achieves the desired effects, as can be determined by one skilled in the art. Selection of a time period between administrations of different viruses can be determined according to parameters similar to those for selecting the time period between administrations of the same virus, including results from monitoring steps, the time period for a subject to mount an immune response, the time period for a subject to clear virus from normal tissue, or the time period for virus proliferation in the tumor or metastasis. In one example, the time period can be a function of the time period for a subject to mount an immune response; for example, the time period can be more than the time period for a subject to mount an immune response, such as more than about one week, more than about ten days, more than about two weeks, or more than about a month; in another example, the time period can be less than the time period for a subject to mount an immune response, such as less than about one week, less than about ten days, less than about two weeks, or less than about a month. In another example, the time period can be a function of the time period for a subject to clear the virus from normal tissue; for example, the time period can be more than the time period for a subject to clear the virus from normal tissue, such as more than about a day, more than about two days, more than about three days, more than about five days, or more than about a week. In another example, the time period can be a function of the time period for virus proliferation in the tumor or metastasis; for example, the time period can be more than the amount of time for a detectable signal to arise in a tumor or metastasis after administration of a virus expressing a detectable marker, such as about 3 days, about 5 days, about a week, about ten days, about two weeks, or about a month.

b. Therapeutic Compounds

Any therapeutic or anti-cancer agent can be used as the second, therapeutic or anti-cancer agent in the combined treatment methods for a stem cell-derived tumor provided herein. The methods can include administering one or more therapeutic compounds to the subject in addition to administering a virus or plurality thereof to a subject having a tumor resulting from a stem cell therapy. Therapeutic compounds can act independently, or in conjunction with the virus, for tumor therapeutic effects.

Therapeutic compounds that can act independently include any of a variety of known chemotherapeutic compounds that can inhibit tumor growth, inhibit metastasis growth and/or formation, decrease the size of a tumor or metastasis, eliminate a tumor or metastasis, without reducing the ability of a virus to accumulate in a tumor, replicate in the tumor, and cause or enhance an anti-tumor immune response in the subject.

Therapeutic compounds that act in conjunction with the viruses include, for example, compounds that alter the expression of the viruses or compounds that can interact with a virally-expressed gene, or compounds that can inhibit virus proliferation, including compounds toxic to the virus. Therapeutic compounds that can act in conjunction with the virus include, for example, therapeutic compounds that increase the proliferation, toxicity, tumor cell killing or immune response eliciting properties of a virus, and also can include, for example, therapeutic compounds that decrease the proliferation, toxicity or cell killing properties of a virus. Optionally, the therapeutic agent can exhibit or manifest additional properties, such as, properties that permit its use as an imaging agent, as described elsewhere herein.

Therapeutic compounds also include, but are not limited to, chemotherapeutic agents, nanoparticles, radiation therapy, siRNA molecules, enzyme/pro-drug pairs, photosensitizing agents, toxins, microwaves, a radionuclide, an angiogenesis inhibitor, a mitosis inhibitor protein (e.g., cdc6), an antitumor oligopeptide (e.g., antimitotic oligopeptides, high affinity tumor-selective binding peptides), a signaling modulator, anti-cancer antibiotics, or a combination thereof.

Exemplary photosensitizing agents include, but are not limited to, for example, indocyanine green, toluidine blue, aminolevulinic acid, texaphyrins, benzoporphyrins, phenothiazines, phthalocyanines, porphyrins such as sodium porfimer, chlorins such as tetra(m-hydroxyphenyl)chlorin or tin(IV) chlorin e6, purpurins such as tin ethyl etiopurpurin, purpurinimides, bacteriochlorins, pheophorbides, pyropheophorbides or cationic dyes. In one embodiment, a vaccinia virus, such as a vaccinia virus provided herein, is administered to a subject having a tumor, cancer or metastasis in combination with a photosensitizing agent.

Radionuclides, which depending up the radionuclide, amount and application can be used for diagnosis and/or for treatment. They include, but are not limited to, for example, a compound or molecule containing ³²Phosphate, ⁶⁰Cobalt, ⁹⁰Yttirum, ⁹⁹Technicium, ¹⁰³Palladium, ¹⁰⁶Ruthenium, ¹¹¹Indium, ¹¹⁷Lutetium, ¹²⁵Iodine, ¹³¹Iodine, ¹³⁷Cesium, ¹⁵³Samarium, ¹⁸⁶Rhenium, ¹⁸⁸Rhenium, ¹⁹²Iridium, ¹⁹⁸Gold, ²¹¹Astatine, ²¹²Bismuth or ²¹³Bismuth. In one embodiment, a vaccinia virus, such as a vaccinia virus provided herein, is administered to a subject having a tumor, cancer or metastasis in combination with a radionuclide.

Toxins include, but are not limited to, chemotherapeutic compounds such as, but not limited to, 5-fluorouridine, calicheamicin and maytansine. Signaling modulators include, but are not limited to, for example, inhibitors of macrophage inhibitory factor, toll-like receptor agonists and stat 3 inhibitors. In one embodiment, a vaccinia virus, such as a vaccinia virus provided herein, is administered to a subject having a tumor, cancer or metastasis in combination with a toxin or a signaling modulator.

Combination therapy between chemotherapeutic agents and oncolytic viruses can be effective/curative in situations when single agent treatment is not effective. Chemotherapeutic compounds include, but are not limited to, alkylating agents such as thiotepa and cyclosphosphamide; alkyl sulfonates such as busulfan, improsulfan and piposulfan; aziridines such as benzodopa, carboquone, meturedopa and uredopa; ethylenimines and methylamelamines including altretamine, triethylenemelamine, trietylenephosphoramide, triethylenethiophosphaoramide and trimethylolomelamime nitrogen mustards such as chiorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; polysaccharide-K; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; cytosine arabinoside; cyclophosphamide; thiotepa; taxoids, e.g., paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT11; topoisomerase inhibitor RFS 2000; difluoromethylornithine (DMFO); retinoic acid; esperamicins; capecitabine; and pharmaceutically acceptable salts, acids or derivatives of any of the above. Also included are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone and toremifene (Fareston); and antiandrogens such as flutamide, nilutamide, bicalutamide, leuprolide and goserelin; and pharmaceutically acceptable salts, acids or derivatives of any of the above. Such chemotherapeutic compounds that can be used herein include compounds whose toxicities preclude use of the compound in general systemic chemotherapeutic methods. Chemotherapeutic agents also include new classes of targeted chemotherapeutic agents such as, for example, imatinib (sold by Novartis under the trade name Gleevec in the United States), gefitinib (developed by Astra Zeneca under the trade name Iressa) and erlotinib. Particular chemotherapeutic agents include, but are not limited to, cisplatin, carboplatin, oxaliplatin, DWA2114R, NK121, IS 3 295, and 254-S vincristine, prednisone, doxorubicin and L-asparaginase; mechoroethamine, vincristine, procarbazine and prednisone (MOPP), cyclophosphamide, vincristine, procarbazine and prednisone (C-MOPP), bleomycin, vinblastine, gemcitabine and 5-fluorouracil. Exemplary chemotherapeutic agents are, for example, cisplatin, carboplatin, oxaliplatin, DWA2114R, NK121, IS 3 295, and 254-S. In a non-limiting embodiment, a vaccinia virus, such as a vaccinia virus provided herein, is administered to a subject having a tumor, cancer or metastasis in combination with a platinum coordination complex, such as cisplatin, carboplatin, oxaliplatin, DWA2114R, NK121, IS 3 295, and 254-S. Tumors, cancers and metastasis can be any of those provided herein, and in particular, can be a pancreatic tumor, an ovarian tumor, a lung tumor, a colon tumor, a prostate tumor, a cervical tumor or a breast tumor; exemplary tumors are pancreatic and ovarian tumors. Tumors, cancers and metastasis can be a monotherapy-resistant tumor such as, for example, one that does not respond to therapy with virus alone or anti-cancer agent alone, but that does respond to therapy with a combination of virus and anti-cancer agent. Typically, a therapeutically effective amount of virus is systemically administered to the subject and the virus localizes and accumulates in the tumor. Subsequent to administering the virus, the subject is administered a therapeutically effective amount of an anti-cancer agent, such as cisplatin. In one example, cisplatin is administered once-daily for five consecutive days. One of skill in the art could determine when to administer the anti-cancer agent subsequent to the virus using, for example, in vivo animal models. Using the methods provided herein, administration of a virus and anti-cancer agent, such as cisplatin can cause a reduction in tumor volume, can cause tumor growth to stop or be delayed or can cause the tumor to be eliminated from the subject. The status of tumors, cancers and metastasis following treatment can be monitored using any of the methods provided herein and known in the art.

Exemplary anti-cancer antibiotics include, but are not limited to, anthracyclines such as doxorubicin hydrochloride (adriamycin), idarubicin hydrochloride, daunorubicin hydrochloride, aclarubicin hydrochloride, epirubicin hydrochloride and purarubicin hydrochloride, pleomycins such as pleomycin and peplomycin sulfate, mitomycins such as mitomycin C, actinomycins such as actinomycin D, zinostatinstimalamer and polypeptides such as neocarzinostatin. In one embodiment, a vaccinia virus, such as a vaccinia virus provided herein, is administered to a subject having a tumor, cancer or metastasis in combination with an anti-cancer antibiotic.

In one embodiment, nanoparticles can be designed such that they carry one or more therapeutic agents provided herein. Additionally, nanoparticles can be designed to carry a molecule that targets the nanoparticle to the tumor cells. In one non-limiting example, nanoparticles can be coated with a radionuclide and, optionally, an antibody immunoreactive with a tumor-associated antigen. In one embodiment, a vaccinia virus, such as a vaccinia virus provided herein, is administered to a subject having a tumor, cancer or metastasis in combination with a nanoparticle carrying any of the therapeutic agents provided herein.

Radiation therapy has become a foremost choice of treatment for a majority of cancer patients. The wide use of radiation treatment stems from the ability of gamma-irradiation to induce irreversible damage in targeted cells with the preservation of normal tissue function. Ionizing radiation triggers apoptosis, the intrinsic cellular death machinery in cancer cells, and the activation of apoptosis seems to be the principal mode by which cancer cells die following exposure to ionizing radiation. In one embodiment, a vaccinia virus, such as a vaccinia virus provided herein, is administered to a subject having a tumor, cancer or metastasis in combination with radiation therapy.

Thus, provided herein are methods of administering to a subject one or more therapeutic compounds that can act in conjunction with the virus to increase the proliferation, toxicity, tumor cell killing, or immune response eliciting properties of a virus. Also provided herein are methods of administering to a subject one or more therapeutic compounds that can act in conjunction with the virus to decrease the proliferation, toxicity, or cell killing properties of a virus. Therapeutic compounds to be administered can be any of those provided herein or in the art.

Therapeutic compounds that can act in conjunction with the virus to increase the proliferation, toxicity, tumor cell killing or immune response eliciting properties of a virus are compounds that can alter gene expression, where the altered gene expression can result in an increased killing of tumor cells or an increased anti-tumor immune response in the subject. A gene expression-altering compound can, for example, cause an increase or decrease in expression of one or more viral genes, including endogenous viral genes and/or exogenous viral genes. For example, a gene expression-altering compound can induce or increase transcription of a gene in a virus such as an exogenous gene that can cause cell lysis or cell death, that can provoke an immune response, that can catalyze conversion of a prodrug-like compound, or that can inhibit expression of a tumor cell gene. Any of a wide variety of compounds that can alter gene expression are known in the art, including IPTG and RU486. Exemplary genes whose expression can be up-regulated include proteins and RNA molecules, including toxins, enzymes that can convert a prodrug to an anti-tumor drug, cytokines, transcription regulating proteins, siRNA and ribozymes. In another example, a gene expression-altering compound can inhibit or decrease transcription of a gene in a virus such as a heterologous gene that can reduce viral toxicity or reduces viral proliferation. Any of a variety of compounds that can reduce or inhibit gene expression can be used in the methods provided herein, including siRNA compounds, transcriptional inhibitors or inhibitors of transcriptional activators. Exemplary genes whose expression can be down-regulated include proteins and RNA molecules, including viral proteins or RNA that suppress lysis, nucleotide synthesis or proliferation, and cellular proteins or RNA molecules that suppress cell death, immunoreactivity, lysis, or viral replication.

In another embodiment, therapeutic compounds that can act in conjunction with the virus to increase the proliferation, toxicity, tumor cell killing, or immune response eliciting properties of a virus are compounds that can interact with a virally expressed gene product, and such interaction can result in an increased killing of tumor cells or an increased anti-tumor immune response in the subject. A therapeutic compound that can interact with a virally-expressed gene product can include, for example a prodrug or other compound that has little or no toxicity or other biological activity in its subject-administered form, but after interaction with a virally expressed gene product, the compound can develop a property that results in tumor cell death, including but not limited to, cytotoxicity, ability to induce apoptosis, or ability to trigger an immune response. In one non-limiting example, the virus carries an enzyme into the cancer cells. Once the enzyme is introduced into the cancer cells, an inactive form of a chemotherapy drug (i.e., a prodrug) is administered. When the inactive prodrug reaches the cancer cells, the enzyme converts the prodrug into the active chemotherapy drug, so that it can kill the cancer cell. Thus, the treatment is targeted only to cancer cells and does not affect normal cells. The prodrug can be administered concurrently with, or sequentially to, the virus. A variety of prodrug-like substances are known in the art and an exemplary set of such compounds are disclosed elsewhere herein, where such compounds can include gancyclovir, 5-fluorouracil, 6-methylpurine deoxyriboside, cephalosporin-doxorubicin, 4-[(2-chloroethyl)(2-mesuloxyethyl)amino]benzoyl-L-glutamic acid, acetaminophen, indole-3-acetic acid, CB1954, 7-ethyl-10-[4-(1-piperidino)-1-piperidino]carbonyloxycamptothecin, bis-(2-chloroethyl)amino-4-hydroxyphenyl-aminomethanone 28, 1-chloromethyl-5-hydroxy-1,2-dihyro-3H-benz[e]indole, epirubicin-glucuronide, 5′-deoxy-5-fluorouridine, cytosine arabinoside, linamarin, and a nucleoside analogue (e.g., fluorouridine, fluorodeoxyuridine, fluorouridine arabinoside, cytosine arabinoside, adenine arabinoside, guanine arabinoside, hypoxanthine arabinoside, 6-mercaptopurineriboside, theoguanosine riboside, nebularine, 5-iodouridine, 5-iododeoxyuridine, 5-bromodeoxyuridine, 5-vinyldeoxyuridine, 9-[(2-hydroxy)ethoxy]methylguanine (acyclovir), 9-[(2-hydroxy-1-hydroxymethyl)-ethoxy]methylguanine (DHPG), azauridien, azacytidine, azidothymidine, dideoxyadenosine, dideoxycytidine, dideoxyinosine, dideoxyguanosine, dideoxythymidine, 3′-deoxyadenosine, 3′-deoxycytidine, 3′-deoxyinosine, 3′-deoxyguanosine, 3′-deoxythymidine).

In another embodiment, therapeutic compounds that can act in conjunction with the virus to decrease the proliferation, toxicity or cell killing properties of a virus are compounds that can inhibit viral replication, inhibit viral toxins or cause viral death. A therapeutic compound that can inhibit viral replication, inhibit viral toxins, or cause viral death can generally include a compound that can block one or more steps in the viral life cycle, including, but not limited to, compounds that can inhibit viral DNA replication, viral RNA transcription, viral coat protein assembly, outer membrane or polysaccharide assembly. Any of a variety of compounds that can block one or more steps in a viral life cycle are known in the art, including any known antiviral compound (e.g., cidofovir), viral DNA polymerase inhibitors, viral RNA polymerase inhibitors, inhibitors of proteins that regulate viral DNA replication or RNA transcription. In another example, a virus can contain a gene encoding a viral life cycle protein, such as DNA polymerase or RNA polymerase that can be inhibited by a compound that is, optionally, non-toxic to the host organism.

In addition to combination therapy between chemotherapeutic agents and a virus provided herein, other more complex combination therapy strategies could be applied as well. For example, a combination therapy can include chemotherapeutic agents, therapeutic antibodies, and a virus provided herein. Alternatively, another combination therapy can be the combination of radiation, therapeutic antibodies, and a virus provided herein. Therefore, the concept of combination therapy also can be based on the application of a virus provided herein virus along with one or more of the following therapeutic modalities, namely, chemotherapeutic agents, radiation therapy, therapeutic antibodies, hyper- or hypothermia therapy, siRNA, diagnostic/therapeutic bacteria, diagnostic/therapeutic mammalian cells, immunotherapy, and/or targeted toxins (delivered by antibodies, liposomes and nanoparticles).

Effective delivery of each components of the combination therapy is an important aspect of the methods provided herein. In accordance with one aspect, the modes of administration discussed below exploit one of more of the key features: (i) delivery of a virus provided herein to the tumors by a mode of administration effect to achieve highest titer of virus and highest therapeutic effect; (ii) delivery of any other mentioned therapeutic modalities to the tumor by a mode of administration to achieve the optimal therapeutic effect. The dose scheme of the combination therapy administered is such that the combination of the two or more therapeutic modalities is therapeutically effective. Dosages will vary in accordance with such factors as the age, health, sex, size and weight of the patient, the route of administration, the toxicity of the drugs, frequency of treatment and the relative susceptibilities of the cancer to each of the therapeutic modalities.

For combination therapies with chemotherapeutic compounds, dosages for the administration of such compounds are known in the art or can be determined by one skilled in the art according to known clinical factors (e.g., subject's species, size, body surface area, age, sex, immunocompetence, and general health, duration and route of administration, the kind and stage of the disease, for example, tumor size, and other viruses, treatments, or compounds, such as other chemotherapeutic drugs, being administered concurrently). In addition to the above factors, such levels can be affected by the infectivity of the virus, and the nature of the virus, as can be determined by one skilled in the art. For example, Cisplatin (also called cis-platinum, platinol; cis-diamminedichloroplatinum; and cDDP) is representative of a broad class of water-soluble, platinum coordination compounds frequently employed in the therapy of testicular cancer, ovarian tumors and a variety of other cancers. (See, e.g., Blumenreich et al. Cancer 55(5): 1118-1122 (1985); Forastiere et al. J. Clin. Oncol. 19(4): 1088-1095 (2001)). Methods of employing cisplatin clinically are well known in the art. For example, cisplatin has been administered in a single day over a six hour period, once per month, by slow intravenous infusion. For localized lesions, cisplatin can be administered by local injection. Intraperitoneal infusion can also be employed. Cisplatin can be administered in doses as low as 10 mg/m2 per treatment if part of a multi-drug regimen, or if the patient has an adverse reaction to higher dosing. In general, a clinical dose is from about 30 to about 120 or 150 mg/m2 per treatment.

Typically, platinum-containing chemotherapeutic agents are administered parenterally, for example by slow intravenous infusion, or by local injection, as discussed above. The effects of intralesional (intra-tumoral) and IP administration of cisplatin is described in (Nagase et al. Cancer Treat. Rep. 71(9): 825-829 (1987); and Theon et al. J. Am. Vet. Med. Assoc. 202(2): 261-7. (1993)).

In one exemplary embodiment, the mutant vaccinia virus is administered once or 2-4 times with 0-60 days apart, followed by 1-30 days where no anti-cancer treatment, then cisplatin is administered daily for 1-5 days, followed by 1-30 days where no anti-cancer treatment is administered. Each component of the therapy, virus or cisplatin treatment, or the virus and cisplatin combination therapy can be repeated. In another exemplary embodiment, cisplatin is administered daily for 1 to 5 days, followed by 1-10 days where no anti-cancer treatment is administered, then the mutant vaccinia virus is administered once or 2-4 times with 0-60 days apart. Such treatment scheme can be repeated. In another exemplary embodiment, cisplatin is administered daily for 1 to 5 days, followed by 1-10 days where no anti-cancer treatment is administered, then the mutant vaccinia virus is administered once or 2-4 times with 0-60 days apart. This is followed by 5-60 days where no anti-cancer treatment is administered, then cisplatin is administered again for 1-5 days. Such treatment scheme can be repeated.

Gemcitabine (GEMZAR®) is another compound employed in the therapy of breast cancer, non-small cell lung cancer, and pancreatic cancer. Gemcitabine is a nucleoside analogue that exhibits antitumor activity. Methods of employing gemcitabine clinically are well known in the art. For example, gemcitabine has been administered by intravenous infusion at a dose of 1000 mg/m2 over 30 minutes once weekly for up to 7 weeks (or until toxicity necessitates reducing or holding a dose), followed by a week of rest from treatment of pancreatic cancer. Subsequent cycles can consist of infusions once weekly for 3 consecutive weeks out of every 4 weeks. Gemcitabine has also been employed in combination with cisplatin in cancer therapy.

In one exemplary embodiment, the mutant vaccinia virus is administered once or 2-4 times with 0-60 days apart, followed by 1-30 days where no anti-cancer treatment is administered, then gemcitabine is administered 1-7 times with 0-30 days apart, followed by 1-30 days where no anti-cancer treatment is administered. Such treatment scheme can be repeated. In another exemplary embodiment, gemcitabine is administered 1-7 times with 0-30 days apart, followed by 1-10 days where no anti-cancer treatment is administered, then the mutant vaccinia virus is administered once or 2-4 times with 0-60 days apart. This is followed by 5-60 days where no anti-cancer treatment is administered. Such treatment scheme can be repeated. In another exemplary embodiment, gemcitabine is administered 1-7 times with 0-30 days apart, followed by 1-10 days where no anti-cancer treatment is administered, then the mutant vaccinia virus is administered once or 2-4 times with 0-60 days apart. This is followed by 5-60 days where no anti-cancer treatment is administered, then gemcitabine is administered again for 1-7 times with 0-30 days apart. Such treatment scheme can be repeated.

As will be understood by one of skill in the art, the optimal treatment regimen will vary and it is within the scope of the treatment methods to evaluate the status of the disease under treatment and the general health of the patient prior to, and following one or more cycles of combination therapy in order to determine the optimal therapeutic combination.

c. Immunotherapies and Biological Therapies

Therapeutic compounds also include, but are not limited to, compounds that exert an immunotherapeutic effect, stimulate the immune system, carry a therapeutic compound, or a combination thereof. Optionally, the therapeutic agent can exhibit or manifest additional properties, such as, properties that permit its use as an imaging agent, as described elsewhere herein. Such therapeutic compounds include, but are not limited to, anti-cancer antibodies, radiation therapy, siRNA molecules and compounds that suppress the immune system. Immunotherapy includes for example, immune-stimulating molecules (protein-based or non-protein-based), cells and antibodies. Immunotherapy treatments can include stimulating immune cells to act more effectively or to make the tumor cells or tumor associated antigens recognizable to the immune system (i.e., break tolerance).

Cytokines and growth factors include, but are not limited to, interleukins, such as, for example, interleukin-1, interleukin-2, interleukin-6 and interleukin-12, tumor necrosis factors, such as tumor necrosis factor alpha (TNF-α), interferons such as interferon gamma (IFN-γ), granulocyte macrophage colony stimulating factors (GM-CSF), angiogenins, and tissue factors.

Anti-cancer antibodies include, but are not limited to, Rituximab, ADEPT, Trastuzumab (Herceptin), Tositumomab (Bexxar), Cetuximab (Erbitux), Ibritumomab (Zevalin), Alemtuzumab (Campath-1H), Epratuzumab (Lymphocide), Gemtuzumab ozogamicin (Mylotarg), Bevacimab (Avastin), Tarceva (Erlotinib), SUTENT (sunitinib malate), Panorex (Edrecolomab), RITUXAN (Rituximab), Zevalin (90Y-ibritumomab tiuexetan), Mylotarg (Gemtuzumab Ozogamicin) and Campath (Alemtuzumab).

Thus, provided herein are methods of administering to a subject one or more therapeutic compounds that can act in conjunction with the virus to stimulate or enhance the immune system, thereby enhancing the effect of the virus. Such immunotherapy can be either delivered as a separate therapeutic modality or could be encoded (if the immunotherapy is protein-based) by the administered virus.

Biological therapies are treatments that use natural body substances or drugs made from natural body substances. They can help to treat a cancer and control side effects caused by other cancer treatments such as chemotherapy. Biological therapies are also sometimes called Biological Response Modifiers (BRM's), biologic agents or simply “biologics” because they stimulate the body to respond biologically (or naturally) to cancer. Immunotherapy is treatment using natural substances that the body uses to fight infection and disease. Because it uses natural substances, immunotherapy is also a biological therapy. There are several types of drugs that come under the term biological therapy: these include, for example, monoclonal antibodies (mAbs), cancer vaccines, growth factors for blood cells, cancer growth inhibitors, anti-angiogenic factors, interferon alpha, interleukin-2 (IL-2), gene therapy and BCG vaccine for bladder cancer

Monoclonal antibodies (mAbs) are of particular interest for treating cancer because of the specificity of binding to a unique antigen and the ability to produce large quantities in the laboratory for mass distribution. Monoclonal antibodies can be engineered to act in the same way as immune system proteins: that is, to seek out and kill foreign matter in your body, such as viruses. Monoclonal antibodies can be designed to recognize epitopes on the surface of cancer cells. The antibodies target specifically bind to the epitopes and either kill the cancer cells or deliver a therapeutic agent to the cancer cell. Methods of conjugating therapeutic agents to antibodies is well-known in the art. Different antibodies have to be made for different types of cancer; for example, Rituximab recognizes CD20 protein on the outside of non Hodgkin's lymphoma cells; ADEPT is a treatment using antibodies that recognize bowel (colon) cancer; and Trastuzumab (Herceptin) recognizes breast cancer cells that produce too much of the protein HER 2 (“HER 2 positive”). Other antibodies include, for example, Tositumomab (Bexxar), Cetuximab (Erbitux), Ibritumomab (Zevalin), Alemtuzumab (Campath-1H), Epratuzumab (Lymphocide), Gemtuzumab ozogamicin (Mylotarg) and Bevacimab (Avastin). Thus, the viruses provided herein can be administered concurrently with, or sequentially to, one or more monoclonal antibodies in the treatment of cancer. In one embodiment, additional therapy is administered in the form of one or more of any of the other treatment modalities provided herein.

5. Monitoring

The methods provided herein can further include one or more steps of monitoring the subject, monitoring the stem-cell implant, stem cell-derived tumor, and/or monitoring the localization of the virus administered to the subject. Any of a variety of monitoring steps can be included in the methods provided herein, including, but not limited to, monitoring tumor size, monitoring anti-(tumor antigen) antibody titer, monitoring the presence and/or size of metastases, monitoring the subject's lymph nodes, monitoring the subject's weight or other health indicators including blood or urine markers, monitoring anti-(viral antigen) antibody titer, monitoring viral expression of a detectable gene product, and directly monitoring viral titer in a tumor, tissue or organ of a subject. In the methods provided herein where the stem cell composition is pretreated with the virus, the progress of the stem cells within the subject also can be monitored by detection of viral expression of a detectable gene product.

The purpose of the monitoring can be simply for assessing the health state of the subject or the progress of therapeutic treatment of the subject, or can be for determining whether or not further administration of the same or a different virus is warranted, or for determining when or whether or not to administer a compound to the subject where the compound can act to increase the efficacy of the therapeutic method, or the compound can act to decrease the pathogenicity of the virus administered to the subject.

a. Monitoring Viral Gene Expression

In some embodiments, the methods provided herein can include monitoring one or more virally expressed genes. Viruses, such as those provided can express one or more detectable gene products, including but not limited to, detectable proteins, such as but not limited to luminescent, fluorescent or proteins that can import detectable molecules into the cell (e.g., transporter proteins). The infected cells in the stem cell composition or stem cell-derived tumor can thus be imaged by one more imaging methods. Measurement of the location of the detectable gene product, for example, by imaging methods including, but not limited to, magnetic resonance, fluorescence, and tomographic methods, can determine the localization of the virus in the subject. Accordingly, the methods provided herein that include monitoring a detectable viral gene product can be used to determine the presence or absence of the virus in one or more organs or tissues of a subject, such as the presence or absence of the virus in a stem cell composition or stem cell-derived tumor or metastases in a subject.

Further, the methods provided herein that include monitoring a detectable viral gene product can be used to determine the titer of virus present in one or more organs, tissues, tumors or metastases. Methods that include monitoring the localization and/or titer of viruses in a subject can be used for determining the pathogenicity of a virus; since viral infection, and particularly the level of infection, of normal tissues and organs can indicate the pathogenicity of the probe, methods of monitoring the localization and/or amount of viruses in a subject can be used to determine the pathogenicity of a virus. Since methods provided herein can be used to monitor the amount of viruses at any particular location in a subject, the methods that include monitoring the localization and/or titer of viruses in a subject can be performed at multiple time points, and, accordingly can determine the rate of viral replication in a subject, including the rate of viral replication in one or more organs or tissues of a subject; accordingly, the methods of monitoring a viral gene product can be used for determining the replication competence of a virus. The methods provided herein also can be used to quantitate the amount of virus present in a variety of organs or tissues, and tumors or metastases, and can thereby indicate the degree of preferential accumulation of the virus in a subject; accordingly, the viral gene product monitoring methods provided herein can be used in methods of determining the ability of a virus to accumulate in tumor or metastases in preference to normal tissues or organs. Since the viruses used in the methods provided herein can accumulate in an entire tumor or can accumulate at multiple sites in a tumor, and can also accumulate in metastases, the methods provided herein for monitoring a viral gene product can be used to determine the size of a tumor or the number of metastases that are present in a subject. Monitoring such presence of viral gene product in tumor or metastasis over a range of time can be used to assess changes in the tumor or metastasis, including growth or shrinking of a tumor, or development of new metastases or disappearance of metastases, and also can be used to determine the rate of growth or shrinking of a tumor, or development of new metastases or disappearance of metastases, or the change in the rate of growth or shrinking of a tumor, or development of new metastases or disappearance of metastases. Accordingly, the methods of monitoring a viral gene product can be used for monitoring a neoplastic disease in a subject, or for determining the efficacy of treatment of a neoplastic disease, by determining rate of growth or shrinking of a tumor, or development of new metastases or disappearance of metastases, or the change in the rate of growth or shrinking of a tumor, or development of new metastases or disappearance of metastases.

Any of a variety of detectable proteins can be detected in the monitoring methods provided herein; an exemplary, non-limiting list of such detectable proteins includes any of a variety of fluorescent proteins (e.g., green or red fluorescent proteins), any of a variety of luciferases, transferrin or other iron binding proteins; or receptors, binding proteins, and antibodies, where a compound that specifically binds the receptor, binding protein or antibody can be a detectable agent or can be labeled with a detectable substance (e.g., a radionuclide or imaging agent). Viruses expressing a detectable protein can be detected by a combination of the method provided herein and know in the art. Viruses expressing more than one detectable protein or two or more viruses expressing various detectable protein can be detected and distinguished by dual imaging methods. For example, a virus expressing a fluorescent protein and an iron binding protein can be detected in vitro or in vivo by low light fluorescence imaging and magnetic resonance, respectively. In another example, a virus expressing two or more fluorescent proteins can be detected by fluorescence imaging at different wavelength. In vivo dual imaging can be performed on a subject that has been administered a virus expressing two or more detectable gene products or two or more viruses each expressing one or more detectable gene products.

b. Monitoring Tumor Size

Also provided herein are methods of monitoring of the size and location of a stem cell-derived tumor. Tumor and or metastasis size can be monitored by any of a variety of methods known in the art, including external assessment methods or tomographic or magnetic imaging methods. In addition to the methods known in the art, methods provided herein, for example, monitoring viral gene expression, can be used for monitoring tumor and/or metastasis size.

Monitoring size over several time points can provide information regarding the increase or decrease in size of a tumor or metastasis, and can also provide information regarding the presence of additional tumors and/or metastases in the subject. Monitoring tumor size over several time points can provide information regarding the development of a neoplastic disease in a subject, including the efficacy of treatment of a neoplastic disease in a subject.

c. Monitoring Antibody Titer

The methods provided herein also can include monitoring the antibody titer in a subject, including antibodies produced in response to administration of a virus to a subject. The viruses administered in the methods provided herein can elicit an immune response to endogenous viral antigens. The viruses administered in the methods provided herein also can elicit an immune response to exogenous genes expressed by a virus. The viruses administered in the methods provided herein also can elicit an immune response to tumor antigens. Monitoring antibody titer against viral antigens, viral expressed exogenous gene products, or tumor antigens can be used in methods of monitoring the toxicity of a virus, monitoring the efficacy of treatment methods, or monitoring the level of gene product or antibodies for production and/or harvesting.

In one embodiment, monitoring antibody titer can be used to monitor the toxicity of a virus. Antibody titer against a virus can vary over the time period after administration of the virus to the subject, where at some particular time points, a low anti-(viral antigen) antibody titer can indicate a higher toxicity, while at other time points a high anti-(viral antigen) antibody titer can indicate a higher toxicity. The viruses used in the methods provided herein can be immunogenic, and can, therefore, elicit an immune response soon after administering the virus to the subject. Generally, a virus against which a subject's immune system can quickly mount a strong immune response can be a virus that has low toxicity when the subject's immune system can remove the virus from all normal organs or tissues. Thus, in some embodiments, a high antibody titer against viral antigens soon after administering the virus to a subject can indicate low toxicity of a virus. In contrast, a virus that is not highly immunogenic can infect a host organism without eliciting a strong immune response, which can result in a higher toxicity of the virus to the host. Accordingly, in some embodiments, a high antibody titer against viral antigens soon after administering the virus to a subject can indicate low toxicity of a virus.

In other embodiments, monitoring antibody titer can be used to monitor the efficacy of treatment methods. In the methods provided herein, antibody titer, such as anti-(tumor antigen) antibody titer, can indicate the efficacy of a therapeutic method such as a therapeutic method to treat neoplastic disease. Therapeutic methods provided herein can include causing or enhancing an immune response against a tumor and/or metastasis. Thus, by monitoring the anti-(tumor antigen) antibody titer, it is possible to monitor the efficacy of a therapeutic method in causing or enhancing an immune response against a tumor and/or metastasis. The therapeutic methods provided herein also can include administering to a subject a virus that can accumulate in a tumor and can cause or enhance an anti-tumor immune response. Accordingly, it is possible to monitor the ability of a host to mount an immune response against viruses accumulated in a tumor or metastasis, which can indicate that a subject has also mounted an anti-tumor immune response, or can indicate that a subject is likely to mount an anti-tumor immune response, or can indicate that a subject is capable of mounting an anti-tumor immune response.

In other embodiments, monitoring antibody titer can be used for monitoring the level of gene product or antibodies for production and/or harvesting. As provided herein, methods can be used for producing proteins, RNA molecules or other compounds by expressing an exogenous gene in a virus that has accumulated in a tumor. Further provided herein are methods for producing antibodies against a protein, RNA molecule or other compound produced by exogenous gene expression of a virus that has accumulated in a tumor. Monitoring antibody titer against the protein, RNA molecule or other compound can indicate the level of production of the protein, RNA molecule or other compound by the tumor-accumulated virus, and also can directly indicate the level of antibodies specific for such a protein, RNA molecule or other compound.

d. Monitoring General Health Diagnostics

The methods provided herein also can include methods of monitoring the health of a subject. Some of the methods provided herein are therapeutic methods, including neoplastic disease therapeutic methods. Monitoring the health of a subject can be used to determine the efficacy of the therapeutic method, as is known in the art. The methods provided herein also can include a step of administering to a subject a virus. Monitoring the health of a subject can be used to determine the pathogenicity of a virus administered to a subject. Any of a variety of health diagnostic methods for monitoring disease such as neoplastic disease, infectious disease, or immune-related disease can be monitored, as is known in the art. For example, the weight, blood pressure, pulse, breathing, color, temperature or other observable state of a subject can indicate the health of a subject. In addition, the presence or absence or level of one or more components in a sample from a subject can indicate the health of a subject. Typical samples can include blood and urine samples, where the presence or absence or level of one or more components can be determined by performing, for example, a blood panel or a urine panel diagnostic test. Exemplary components indicative of a subject's health include, but are not limited to, white blood cell count, hematocrit, or reactive protein concentration.

e. Monitoring Coordinated with Treatment

Also provided herein are methods of monitoring a therapy, where therapeutic decisions can be based on the results of the monitoring. Therapeutic methods provided herein can include administering to a subject a virus, where the virus can preferentially accumulate in a tumor and/or metastasis, and where the virus can cause or enhance an anti-tumor immune response. Such therapeutic methods can include a variety of steps including multiple administrations of a particular virus, administration of a second virus, or administration of a therapeutic compound. Determination of the amount, timing or type of virus or compound to administer to the subject can be based on one or more results from monitoring the subject. For example, the antibody titer in a subject can be used to determine whether or not it is desirable to administer a virus or compound, the quantity of virus or compound to administer, and the type of virus or compound to administer, where, for example, a low antibody titer can indicate the desirability of administering additional virus, a different virus, or a therapeutic compound such as a compound that induces viral gene expression. In another example, the overall health state of a subject can be used to determine whether or not it is desirable to administer a virus or compound, the quantity of virus or compound to administer, and the type of virus or compound to administer, where, for example, determining that the subject is healthy can indicate the desirability of administering additional virus, a different virus, or a therapeutic compound such as a compound that induces viral gene expression. In another example, monitoring a detectable virally expressed gene product can be used to determine whether or not it is desirable to administer a virus or compound, the quantity of virus or compound to administer, and the type of virus or compound to administer. Such monitoring methods can be used to determine whether or not the therapeutic method is effective, whether or not the therapeutic method is pathogenic to the subject, whether or not the virus has accumulated in a tumor or metastasis, and whether or not the virus has accumulated in normal tissues or organs. Based on such determinations, the desirability and form of further therapeutic methods can be derived.

In one embodiment, determination of whether or not a therapeutic method is effective can be used to derive further therapeutic methods. Any of a variety of methods of monitoring can be used to determine whether or not a therapeutic method is effective, as provided herein or otherwise known in the art. If monitoring methods indicate that the therapeutic method is effective, a decision can be made to maintain the current course of therapy, which can include further administrations of a virus or compound, or a decision can be made that no further administrations are required. If monitoring methods indicate that the therapeutic method is ineffective, the monitoring results can indicate whether or not a course of treatment should be discontinued (e.g., when a virus is pathogenic to the subject), or changed (e.g., when a virus accumulates in a tumor without harming the host organism, but without eliciting an anti-tumor immune response), or increased in frequency or amount (e.g., when little or no virus accumulates in tumor).

In one example, monitoring can indicate that a virus is pathogenic to a subject. In such instances, a decision can be made to terminate administration of the virus to the subject, to administer lower levels of the virus to the subject, to administer a different virus to a subject, or to administer to a subject a compound that reduces the pathogenicity of the virus. In one example, administration of a virus that is determined to be pathogenic can be terminated. In another example, the dosage amount of a virus that is determined to be pathogenic can be decreased for subsequent administration; in one version of such an example, the subject can be pre-treated with another virus that can increase the ability of the pathogenic virus to accumulate in tumor, prior to re-administering the pathogenic virus to the subject. In another example, a subject can have administered thereto a virus that is pathogenic to the subject; administration of such a pathogenic virus can be accompanied by administration of, for example, an antiviral compound (e.g., cidofovir), pathogenicity attenuating compound (e.g., a compound that down-regulates the expression of a lytic or apoptotic gene product), or other compound that can decrease the proliferation, toxicity, or cell killing properties of a virus, as described herein elsewhere. In one variation of such an example, the localization of the virus can be monitored, and, upon determination that the virus is accumulated in tumor and/or metastases but not in normal tissues or organs, administration of the antiviral compound or pathogenicity attenuating compound can be terminated, and the pathogenic activity of the virus can be activated or increased, but limited to the tumor and/or metastasis. In another variation of such an example, after terminating administration of the antiviral compound or pathogenicity attenuating compound, the presence of the virus and/or pathogenicity of the virus can be further monitored, and administration of such a compound can be reinitiated if the virus is determined to pose a threat to the host by, for example, spreading to normal organs or tissues, releasing a toxin into the vasculature, or otherwise having pathogenic effects reaching beyond the tumor or metastasis.

In another example, monitoring can determine whether or not a virus has accumulated in a tumor or metastasis of a subject. Upon such a determination, a decision can be made to further administer additional virus, a different virus or a compound to the subject. In another example, monitoring the presence of a virus in a tumor can be used in deciding to administer to the subject a compound, where the compound can increase the pathogenicity, proliferation, or immunogenicity of a virus or the compound can otherwise act in conjunction with the virus to increase the proliferation, toxicity, tumor cell killing, or immune response eliciting properties of a virus; in one variation of such an example, the virus can, for example, have little or no lytic or cell killing capability in the absence of such a compound; in a further variation of such an example, monitoring of the presence of the virus in a tumor or metastasis can be coupled with monitoring the absence of the virus in normal tissues or organs, where the compound is administered if the virus is present in tumor or metastasis and not at all present or substantially not present in normal organs or tissues; in a further variation of such an example, the amount of virus in a tumor or metastasis can be monitored, where the compound is administered if the virus is present in tumor or metastasis at sufficient levels.

### E. THERAPEUTIC AND DIAGNOSTIC METHODS FOR THE TREATMENT AND PREVENTION OF TUMORS ASSOCIATED WITH OTHER CELL THERAPIES

The therapeutic methods provided herein for the treatment and prevention of stem cell-derived tumors can be applied for treating and/or preventing the formation of tumors and metastases resulting from other cellular therapies. A variety of therapies are known in art, which involve the administration of cellular compositions to a subject for the treatment of a disease or disorder. Exemplary of such cell therapies include administration of cellular compositions containing, for example, immune cells, such as, but not limited to T lymphocytes (including Th1 cells, Th2 cells, tumor infusing lymphocytes (TIL) and cytotoxic T cells (CTL)) antigen presenting cells (APC) (including dendritic cells (DC) and macrophages, and natural killer (NK) cells) and non-immune cells including, but not limited to, neuronal, skin, adrenal, keratinocyte, blood, endothelial, kidney, bone, muscle, heart, retinal, pancreas and liver cells. Because these therapies involve administration of live cells, there is a risk of causing tumors in the subject. For example, the cellular compositions may contain neoplastic cells or neoplastic progenitor cells that can potentially form tumors in the subject following administration of the cellular composition.

Accordingly, provided herein are methods that provide for the safe administration of cell therapies in addition to the stem cell therapies outlined herein. Such methods can be employed to inhibit or treat complications of cell therapy-associated tumor formation that result from administration of cellular compositions to a subject for therapy of a disease or disorder. Exemplary diseases or disorders for which the methods provided herein can be employed include, but are not limited to, cancers, infections and immunodeficiency disorders.

The therapeutic methods provided herein include, but are not limited to, administering a cellular therapy, such as the administration of a cell composition (e.g., an immune cell composition) to a subject, in combination with an oncolytic virus provided herein to prevent tumor formation or to treat a tumor resulting from or generated by the administered cell composition. In some examples, the virus is administered to a subject having a tumor, where the tumor is caused by the cell therapy (i.e., a cell therapy-associated tumor). In other examples, the virus is administered to a subject who is receiving or has received a cell therapy for the prevention of tumor formation.

In some examples, the virus is administered concurrently with administration of a cellular composition. In other examples, the virus is administered at a selected time point following administration of a cellular composition. In other examples, the cellular composition is contacted with the virus prior to administration of the cellular composition to the subject in order to eliminate neoplastic cells from the cellular composition. In some examples, the cellular composition is pretreated with the virus prior to administration of the cellular composition to the subject and the same or different oncolytic virus also is administered concurrently with or subsequent to the administration of the pre-treated cellular composition. The viruses can be administered for diagnosis and/or therapy of subjects, such as, but not limited to humans and other mammals, including rodents, dogs, cats, primates, or livestock.

The viruses for administration can be any of the viruses described and provided herein that are employed in the methods of treatment and prevention of cell therapy-associated tumors. For example, the viruses are typically attentuated viruses that preferentially accumulate in neoplastic cells, including tumors or metastases. Generally, the administered viruses are replication competent viruses and have the ability to preferentially replicate in tumor cells and/or lyse the tumor cells (i.e., oncolytic viruses). In particular examples, the virus is a vaccinia virus, such as, for example, a Lister strain vaccinia virus (e.g., LIVP).

In some examples, the viruses can express one or more genes whose products are useful for tumor therapy. For example, a virus can express a proteins cause cell death or whose products cause an anti-tumor immune response. Exemplary proteins useful for tumor therapy include, but are not limited to, tumor suppressors, toxins, cytostatic proteins, antiangiogenic proteins, antitumor antibodies, and costimulatory molecules, such as cytokines and chemokines among others provided elsewhere herein and known in the art.

In some examples, the viruses also can express one or more genes whose products are useful for tumor detection and/or imaging. Exemplary gene products for imaging or detection include detectable proteins or proteins that induce detectable signals, such as, for example luciferases, fluorescent proteins, receptors that can bind imaging agents, or proteins linked to imaging or diagnostic moieties.

In some exemplary methods, the virus is administered directly to a subject that is administered a cellular composition for the treatment of a disease or disorder. The administered virus accumulates in and infects the administered cellular composition in vivo and can eliminate neoplastic cells or neoplastic progenitor cells of the administered cellular composition in vivo. The oncolytic virus provided herein can be administered concurrently, sequentially, or intermittently with the cellular composition. For example, the virus can be administered to the subject following the administration of the cellular composition, such as, for example, about or 5 minutes, 10 minutes, 15 minutes, 30 minutes, 1 hour, 2 hours, 3 hours, 4 hours, 5 hours, 6 hours, 12 hours, 24 hours, 30 hours, 36 hours, 42 hours, 48 hours, 3 days, 4 days, 5 days, 6 days, 7 days, 8 days, 9 days, 10 days, 11 days, 12 days, 13 days, 14 days, 2 weeks, 3 weeks, 4 weeks, 5 week, 6 weeks or more following the administration of the cellular composition. The cellular composition and/or the virus also can be administered multiple times as described herein, which also are applicable to this method.

The virus and the cellular composition can be administered in the same composition or in separate compositions. The virus and the cellular composition can be administered via the same route of administration or different routes of administration as provided herein. For example, the cellular composition and the virus can be administered locally or systemically, together in the same composition or in separate compositions, via the same route of administration or different routes of administration.

Provided are methods of administering a cellular composition that has been pre-treated with an oncolytic virus for the treatment of a disease or disorder in a subject for which cellular therapy is administered. In such methods, the cellular composition to be administered is first contacted with the virus to permit infection of neoplastic cells or neoplastic progenitor cells in the cellular composition. The virus can preferentially infect and replicate in neoplastic cells and neoplastic progenitor cells and eliminate such cells from the cellular composition. At a selected time point following infection, the pre-treated cellular composition is administered to a subject for the treatment of a disease or disorder to be treated by cellular therapy. The infection time selected should be sufficient to permit infection of the cellular composition. In some examples, the cellular composition is contacted with the virus for 1, 2, 3, 4, 5, 6, 7, 8, 9, 10, 11, 12, 18, 24, 30, 36, 42, 48, or 72 hours or more prior to administration of the pretreated cellular composition.

Provided are methods of administering an oncolytic virus for the treatment of a subject having a tumor resulting from a cellular therapy. In such methods, a subject selected for administration of the virus is one who has been diagnosed as having a tumor following administration of a cellular therapy. In some examples, as described herein, a virus, such as the viruses described herein, can be employed for detection and imaging of the cell therapy-associated tumor in a subject suspected of having a tumor and also can be employed for the treatment of the tumor. In addition, as described herein, such viruses can be employed for the monitoring of treatment of the tumor.

Any mode of administration of a cellular composition can be employed provided the mode of administration permits the cellular composition to treat the disease or disorder in the subject for which cellular therapy is administered. Modes of administration of cells for cellular therapy are known in the art. One skilled in the art can select any mode of administration compatible with the subject and the disease or disorder to be treated.

Any mode of administration of an oncolytic virus to a subject can be used, provided the mode of administration permits the virus to infect neoplastic cells of the administered cellular composition or tumor cells of the cell therapy-associated tumor. Modes of administration can include, but are not limited to, systemic, intravenous, intraperitoneal, subcutaneous, intramuscular, transdermal, intradermal, intra-arterial (e.g., hepatic artery infusion), intravesicular perfusion, intrapleural, intraarticular, topical, intratumoral, intralesional, multipuncture (e.g., as used with smallpox vaccines), inhalation, percutaneous, subcutaneous, intranasal, intratracheal, oral, intracavity (e.g., administering to the bladder via a catheter, administering to the gut by suppository or enema), vaginal, rectal, intracranial, intraprostatic, intravitreal, aural, or ocular administration. One skilled in the art can select any mode of administration compatible with the subject and the virus, and that also is likely to result in the virus reaching the cell therapy implant or cell therapy-associated tumor.

### F. PHARMACEUTICAL COMPOSITIONS, COMBINATIONS AND KITS

Provided herein are pharmaceutical compositions, combinations and kits containing a virus and/or cellular composition provided herein and one or more components. Pharmaceutical compositions can include a virus and/or cellular composition provided herein and a pharmaceutical carrier. Combinations can include two or more viruses, one or more viruses and a cellular composition, a virus and a detectable compound, a virus and a viral expression modulating compound, a virus and a therapeutic compound, or any combination thereof. Kits can include the pharmaceutical compositions and/or combinations provided herein, and one or more components, such as instructions for use, a device for detecting a virus in a subject, a device for administering a compound to a subject, and a device for administering a compound to a subject.

1. Pharmaceutical Compositions

Provided herein are pharmaceutical compositions containing a virus provided herein and a suitable pharmaceutical carrier. Pharmaceutical compositions provided herein can be in various forms, e.g., in solid, liquid, powder, aqueous, or lyophilized form. Examples of suitable pharmaceutical carriers are known in the art and include but are not limited to water, buffers, saline solutions, phosphate buffered saline solutions, various types of wetting agents, sterile solutions, alcohols, gum arabic, vegetable oils, benzyl alcohols, gelatin, glycerin, carbohydrates such as lactose, sucrose, amylose or starch, magnesium stearate, talc, silicic acid, viscous paraffin, perfume oil, fatty acid monoglycerides and diglycerides, pentaerythritol fatty acid esters, hydroxy methylcellulose, powders, among others. Pharmaceutical compositions provided herein can contain other additives including, for example, antioxidants and preservatives, analgesic agents, binders, disintegrants, coloring, diluents, excipients, extenders, glidants, solubilizers, stabilizers, tonicity agents, vehicles, viscosity agents, flavoring agents, emulsions, such as oil/water emulsions, emulsifying and suspending agents, such as acacia, agar, alginic acid, sodium alginate, bentonite, carbomer, carrageenan, carboxymethylcellulose, cellulose, cholesterol, gelatin, hydroxyethyl cellulose, hydroxypropyl cellulose, hydroxypropyl methylcellulose, methylcellulose, octoxynol 9, oleyl alcohol, povidone, propylene glycol monostearate, sodium lauryl sulfate, sorbitan esters, stearyl alcohol, tragacanth, xanthan gum, and derivatives thereof, solvents, and miscellaneous ingredients such as crystalline cellulose, microcrystalline cellulose, citric acid, dextrin, dextrose, liquid glucose, lactic acid, lactose, magnesium chloride, potassium metaphosphate, starch, among others. Such carriers and/or additives can be formulated by conventional methods and can be administered to the subject at a suitable dose. Stabilizing agents such as lipids, nuclease inhibitors, polymers, and chelating agents can preserve the compositions from degradation within the body.

Colloidal dispersion systems that can be used for delivery of viruses include macromolecule complexes, nanocapsules, microspheres, beads and lipid-based systems including oil-in-water emulsions (mixed), micelles, liposomes and lipoplexes. An exemplary colloidal system is a liposome. Organ-specific or cell-specific liposomes can be used in order to achieve delivery only to the desired tissue. The targeting of liposomes can be carried out by the person skilled in the art by applying commonly known methods. This targeting includes passive targeting (utilizing the natural tendency of the liposomes to distribute to cells of the RES in organs which contain sinusoidal capillaries) or active targeting (for example, by coupling the liposome to a specific ligand, for example, an antibody, a receptor, sugar, glycolipid and protein by methods know to those of skill in the art). In the present methods, monoclonal antibodies can be used to target liposomes to specific tissues, for example, tumor tissue, via specific cell-surface ligands.

Pharmaceutical compositions provided herein include of cellular compositions for administration. Pharmaceutical compositions for the storage of cells for subsequent administration to a subject are known in the art and can contain suitable carriers and/or buffers appropriate for the preservation of the cells prior to administration.

2. Combinations

Provided are combinations of the viruses provided herein and a second agent, such as a second virus or other therapeutic or diagnostic agent. A combination can include any virus or reagent for effecting attenuation thereof in accord with the methods provided herein. Combinations can include a virus provided herein with one or more additional viruses. Combinations of the viruses provided can also contain pharmaceutical compositions containing the viruses or host cells containing the viruses as described herein.

In one embodiment, the virus in a combination is an attenuated virus, such as for example, an attenuated vaccinia virus. Exemplary attenuated viruses include vaccinia viruses provided herein, such as, but not limited to, for example, vaccinia viruses described in the Examples: GLV-1h86, GLV-1j87, GLV-1j88, GLV-1j89, GLV-1h90, GLV-1h91, GLV-1h92, GLV-1h96, GLV-1h97, GLV-1h98, GLV-1h104, GLV-1h105, GLV-1h106, GLV-1h107, GLV-1h108 and GLV-1h109.

Combinations provided herein can contain a virus and a therapeutic compound. Therapeutic compounds for the compositions provided herein can be, for example, an anti-cancer or chemotherapeutic compound. Exemplary therapeutic compounds include, for example, cytokines, growth factors, photosensitizing agents, radionuclides, toxins, siRNA molecules, enzyme/pro-drug pairs, anti-metabolites, signaling modulators, anti-cancer antibiotics, anti-cancer antibodies, angiogenesis inhibitors, chemotherapeutic compounds, or a combination thereof. Viruses provided herein can be combined with an anti-cancer compound, such as a platinum coordination complex. Exemplary platinum coordination complexes include, for example, cisplatin, carboplatin, oxaliplatin, DWA2114R, NK121, IS 3 295, and 254-S. Additional exemplary therapeutic compounds for the use in pharmaceutical composition combinations can be found elsewhere herein (see e.g., Section I. THERAPEUTIC METHODS for exemplary cytokines, growth factors, photosensitizing agents, radionuclides, toxins, siRNA molecules, enzyme/pro-drug pairs, anti-metabolites, signaling modulators, anti-cancer antibiotics, anti-cancer antibodies, angiogenesis inhibitors, and chemotherapeutic compounds). Exemplary chemotherapeutic agents include methotrexate, vincristine, adriamycin, non-sugar containing chloroethylnitrosoureas, 5-fluorouracil, mitomycin C, bleomycin, doxorubicin, dacarbazine, taxol, fragyline, Meglamine GLA, valrubicin, carmustaine and poliferposan, MM1270, BAY 12-9566, RAS farnesyl transferase inhibitor, farnesyl transferase inhibitor, MMP, MTA/LY231514, LY264618/Lometexol, Glamolec, CI-994, TNP-470, Hycamtin/Topotecan, PKC412, Valspodar/PSC833, Novantrone/Mitroxantrone, Metaret/Suramin, Batimastat, E7070, BCH-4556, CS-682, 9-AC, AG3340, AG3433, Incel/VX-710, VX-853, ZD0101, IS1641, ODN 698, TA 2516/Marmistat, BB2516/Marmistat, CDP 845, D2163, PD183805, DX8951f, Lemonal DP 2202, FK 317, Picibanil/OK-432, AD 32/Valrubicin, Metastron/strontium derivative, Temodal/Temozolomide, Evacet/liposomal doxorubicin, Yewtaxan/Placlitaxel, Taxol/Paclitaxel, Xeload/Capecitabine, Furtulon/Doxifluridine, Cyclopax/oral paclitaxel, Oral Taxoid, SPU-077/Cisplatin, HMR 1275/Flavopiridol, CP-358 (774)/EGFR, CP-609 (754)/RAS oncogene inhibitor, BMS-182751/oral platinum, UFT(Tegafur/Uracil), Ergamisol/Levamisole, Eniluracil/776C85/5FU enhancer, Campto/Levamisole, Camptosar/Irinotecan, Tumodex/Ralitrexed, Leustatin/Cladribine, Paxex/Paclitaxel, Doxil/liposomal doxorubicin, Caelyx/liposomal doxorubicin, Fludara/Fludarabine, Pharmarubicin/Epirubicin, DepoCyt, ZD1839, LU 79553/Bis-Naphtalimide, LU 103793/Dolastain, Caetyx/liposomal doxorubicin, Gemzar/Gemcitabine, ZD 0473/Anormed, YM 116, Iodine seeds, CDK4 and CDK2 inhibitors, PARP inhibitors, D4809/Dexifosamide, Ifes/Mesnex/Ifosamide, Vumon/Teniposide, Paraplatin/Carboplatin, Plantinol/cisplatin, Vepeside/Etoposide, ZD 9331, Taxotere/Docetaxel, prodrug of guanine arabinoside, Taxane Analog, nitrosoureas, alkylating agents such as melphelan and cyclophosphamide, Aminoglutethimide, Asparaginase, Busulfan, Carboplatin, Chlorombucil, Cytarabine HCl, Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erythropoietin, Hexamethylmelamine (HMM), Interleukin 2, Mitoguazone (methyl-GAG; methyl glyoxal bis-guanylhydrazone; MGBG), Pentostatin (2′ deoxycoformycin), Semustine (methyl-CCNU), Teniposide (VM-26) and Vindesine sulfate.

In a further embodiment, the combination can include additional therapeutic compounds such as, for example, compounds that are substrates for enzymes encoded and expressed by the virus, or other therapeutic compounds provided herein or known in the art to act in concert with a virus. For example, the virus can express an enzyme that converts a prodrug into an active chemotherapy drug for killing the cancer cell. Hence, combinations provided herein can contain therapeutic compounds, such as prodrugs. An exemplary virus/therapeutic compound combination can include a virus encoding Herpes simplex virus thymidine kinase with the prodrug gancyclovir. Additional exemplary enzyme/pro-drug pairs, for the use in combinations provided include, but are not limited to, varicella zoster thymidine kinase/gancyclovir, cytosine deaminase/5-fluorouracil, purine nucleoside phosphorylase/6-methylpurine deoxyriboside, beta lactamase/cephalosporin-doxorubicin, carboxypeptidase G2/4-[(2-chloroethyl)(2-mesuloxyethyl)amino]benzoyl-L-glutamic acid, cytochrome P450/acetominophen, horseradish peroxidase/indole-3-acetic acid, nitroreductase/CB 1954, rabbit carboxylesterase/7-ethyl-10-[4-(1-piperidino)-1-piperidino]carbonyloxycampotothecin, mushroom tyrosinase/bis-(2-chloroethyl)amino-4-hydroxyphenylaminomethanone 28, beta galactosidase/1-chloromethyl-5-hydroxy-1,2-dihyro-3H-benz[e]indole, beta glucuronidase/epirubicin-glucoronide, thymidine phosphorylase/5′-deoxy-5-fluorouridine, deoxycytidine kinase/cytosine arabinoside, beta-lactamase and linamerase/linamarin. Additional exemplary prodrugs, for the use in combinations can also be found elsewhere herein (see e.g., Section I. THERAPEUTIC METHODS). Any of a variety of known combinations provided herein or otherwise known in the art can be included in the combinations provided herein.

In a further embodiment, combinations can include compounds that can kill or inhibit viral growth or toxicity. Combinations provided herein can contain antibiotic, antifungal, anti-parasitic or antiviral compounds for treatment of infections. Exemplary antibiotics which can be included in a combination with a virus provided herein include, but are not limited to, ceftazidime, cefepime, imipenem, aminoglycoside, vancomycin, and antipseudomonal β-lactam. Exemplary antifungal agents which can be included in a combination with a virus provided herein include, but are not limited to, amphotericin B, dapsone, fluconazole, flucytosine, griseofluvin, intraconazole, ketoconazole, miconazole, clotrimazole, nystatin, and combinations thereof. Exemplary antiviral agents can be included in a combination with a virus provided herein include, but are not limited to, cidofovir, alkoxyalkyl esters of cidofovir (CDV), cyclic CDV, and (S)-9-(3-hydroxy-2 phosphonylmethoxypropyl)adenine, 5-(Dimethoxymethyl)-2′-deoxyuridine, isatin-beta-thiosemicarbazone, N-methanocarbathymidine, brivudin, 7-deazaneplanocin A, ST-246, Gleevec, 2′-beta-fluoro-2′,3′-dideoxyadenosine, indinavir, nelfinavir, ritonavir, nevirapine, AZT, ddI, ddC, and combinations thereof. Typically, combinations with an antiviral agent contain an antiviral agent known to be effective against the virus of the combination. For example, combinations can contain a vaccinia virus with an antiviral compound, such as cidofovir, alkoxyalkyl esters of cidofovir, gancyclovir, acyclovir, ST-246, and Gleevec.

In another embodiment, the combination can further include a detectable compound. A detectable compound can include a ligand or substrate or other compound that can interact with and/or bind specifically to a virally expressed protein or RNA molecule, and can provide a detectable signal, such as a signal detectable by tomographic, spectroscopic, magnetic resonance, or other known techniques. Exemplary detectable compounds can be, or can contain, an imaging agent such as a magnetic resonance, ultrasound or tomographic imaging agent, including a radionuclide. The detectable compound can include any of a variety of compounds as provided elsewhere herein or are otherwise known in the art. Typically, the detectable compound included with a virus in the combinations provided herein will be a compound that is a substrate, a ligand, or can otherwise specifically interact with, a protein or RNA encoded by the virus; in some examples, the protein or RNA is an exogenous protein or RNA. Exemplary viruses/detectable compounds include a virus encoding luciferase/luciferin, β-galactosidase/(4,7,10-tri(acetic acid)-1-(2-β-galactopyranosylethoxy)-1,4,7,10-tetraazacyclododecane) gadolinium (Egad), and other combinations known in the art.

In another embodiment, the combination can further include a virus gene expression modulating compound. Compounds that modulate gene expression are known in the art, and include, but are not limited to, transcriptional activators, inducers, transcriptional suppressors, RNA polymerase inhibitors, and RNA binding compounds such as siRNA or ribozymes. Any of a variety of gene expression modulating compounds known in the art can be included in the combinations provided herein. Typically, the gene expression modulating compound included with a virus in the combinations provided herein will be a compound that can bind, inhibit, or react with one or more compounds, active in gene expression such as a transcription factor or RNA of the virus of the combination. An exemplary virus/expression modulator can be a virus encoding a chimeric transcription factor complex having a mutant human progesterone receptor fused to a yeast GAL4 DNA-binding domain an activation domain of the herpes simplex virus protein VP16 and also containing a synthetic promoter containing a series of GAL4 recognition sequences upstream of the adenovirus major late E1B TATA box, where the compound can be RU486 (see, e.g., Yu et al., (2002) Mol Genet Genomics 268:169-178). A variety of other virus/expression modulator combinations known in the art also can be included in the combinations provided herein.

In a further embodiment, combination can further contain nanoparticles. Nanoparticles can be designed such that they carry one or more therapeutic agents provided herein. Additionally, nanoparticles can be designed to carry a molecule that targets the nanoparticle to the tumor cells. In one non-limiting example, nanoparticles can be coated with a radionuclide and, optionally, an antibody immunoreactive with a tumor-associated antigen.

3. Kits

The viruses, cell compositions, pharmaceutical compositions or combinations thereof provided herein can be packaged as kits. Kits can optionally include one or more components such as instructions for use, devices, and additional reagents, and components, such as tubes, containers and syringes for practice of the methods. Exemplary kits can include the viruses provided herein, and can optionally include instructions for use, a device for detecting a virus in a subject, a device for administering the virus to a subject, and a device for administering a compound to a subject.

In one example, a kit can contain instructions. Instructions typically include a tangible expression describing the virus and/or cellular composition and, optionally, other components included in the kit, and methods for administration, including methods for determining the proper state of the subject, the proper dosage amount, and the proper administration method, for administering the virus and/or cellular composition. Instructions can also include guidance for monitoring the subject over the duration of the treatment time.

In another example, a kit can contain a device for detecting a virus in a subject. Devices for detecting a virus in a subject can include a low light imaging device for detecting light, for example, emitted from luciferase, or fluoresced from fluorescent protein, such as a green or red fluorescent protein, a magnetic resonance measuring device such as an MRI or NMR device, a tomographic scanner, such as a PET, CT, CAT, SPECT or other related scanner, an ultrasound device, or other device that can be used to detect a protein expressed by the virus within the subject. Typically, the device of the kit will be able to detect one or more proteins expressed by the virus of the kit. Any of a variety of kits containing viruses and detection devices can be included in the kits provided herein, for example, a virus expressing luciferase and a low light imager, or a virus expressing fluorescent protein, such as a green or red fluorescent protein, and a low light imager.

Kits provided herein also can include a device for administering a virus and/or a cellular composition to a subject. Kits provided herein also can include a device for administering a therapeutic compound to a subject. Any of a variety of devices known in the art for administering medications or vaccines can be included in the kits provided herein. Exemplary devices include, but are not limited to, a hypodermic needle, an intravenous needle, a catheter, a needle-less injection device, an inhaler, and a liquid dispenser, such as an eyedropper. Typically, the device for administering a virus and/or stem cell composition of the kit will be compatible with the virus and/or stem cell composition of the kit; for example, a needle-less injection device such as a high pressure injection device can be included in kits with viruses not damaged by high pressure injection, but is typically not included in kits with viruses damaged by high pressure injection.

### G. EXAMPLES

The following examples are included for illustrative purposes only and are not intended to limit the scope of the invention.

### Example 1

### Generation of Modified Vaccinia Virus Strains

### A. Construction of Modified Vaccinia Viruses

1. Construction of GLV-1h68 and GLV-1h22 Strains

Exemplary viruses for use in for use in the methods provided were generated by modification of the vaccinia virus strain designated LIVP (a vaccinia virus strain, originally derived by adapting the Lister strain (ATCC Catalog No. VR-1549) to calf skin (Institute for Research on Virus Preparations, Moscow, Russia, Al'tshtein et al. (1983) Dokl. Akad. Nauk USSR 285:696-699). The LIVP strain (whose genome sequence is set forth in SEQ ID NO: 2) from which the viral strains were generated contains a mutation in the coding sequence of the thymidine kinase (TK) gene in which a substitution of a guanine nucleotide with a thymidine nucleotide (nucleotide position 80207 of SEQ ID NO: 2) introduces a premature STOP codon within the coding sequence. The LIVP strain was further modified to generate the GLV-1h68 virus (SEQ ID NO: 1; U.S. Patent Publication No. 2005-0031643 and Japanese Patent No. 3,934,673).

As described in U.S. Patent Publication No. 2005/0031643 and Japanese Patent No. 3,934,673 (see particularly Example 1 in each application), GLV-1h68 (also named RVGL21, SEQ ID NO: 1) was generated by inserting expression cassettes encoding detectable marker proteins into the F14.5L (also designated in LIVP as F3) gene, thymidine kinase (TK) gene, and hemagglutinin (HA) gene loci of the vaccinia virus LIVP strain. All cloning steps were performed using vaccinia DNA homology-based shuttle plasmids generated for homologous recombination of foreign genes into target loci in the vaccinia virus genome through double reciprocal crossover (see Timiryasova et al. (2001) BioTechniques 31(3) 534-540). As described in U.S. Patent Publication 2005/0031643 and Japanese Patent No. 3,934,673, the GLV-1h68 virus was constructed using plasmids pSC65 (Chakrabarti et al. (1997) Biotechniques 23:1094-1097) and pVY6 (Flexner et al. (1988) Virology 166:339-349) to direct insertions into the TK and HA loci of LIVP genome, respectively. Recombinant viruses were generated by transformation of shuttle plasmid vectors using the FuGENE 6 transfection reagent (Roche Applied Science, Indianapolis, Ind.) into CV-1 cells (ATCC Cat No. CR1-1469), which were pre-infected with the LIVP parental virus, or one of its recombinant derivatives.

The expression cassettes were inserted in the LIVP genome in three separate rounds of recombinant virus production. In the first round, an expression cassette containing a Ruc-GFP cDNA (a fusion of DNA encoding Renilla luciferase and DNA encoding GFP) under the control of a vaccinia synthetic early/late promoter P_{SEL} was inserted into the Not I site of the F14.5L gene locus. In the second round, the resulting recombinant virus from the first round was further modified by insertion of an expression cassette containing DNA encoding beta-galactosidase (LacZ) under the control of the vaccinia early/late promoter P_{7.5k} (denoted (P_{7.5k})lacZ) and DNA encoding a rat transferrin receptor positioned in the reverse orientation for transcription relative to the vaccinia synthetic early/late promoter P_{SEL} (denoted (P_{SEL})rTrfR) was inserted into the TK gene (the resulting virus does not express transferrin receptor protein since the DNA encoding the protein is positioned in the reverse orientation for transcription relative to the promoter in the cassette). In the third round, the resulting recombinant virus from the second round was then further modified by insertion of an expression cassette containing DNA encoding β-glucuronidase under the control of the vaccinia late promoter P₁₁ₖ (denoted (P₁₁ₖ)gusA) was inserted into the HA gene. The resulting virus containing all three insertions is designated GLV-1h68. The complete sequence of GLV-1h68 is shown in SEQ ID NO: 1.

Another genetically engineered vaccinia strain, designated GLV-1h22 (SEQ ID NO: 3) was produced that has essentially the same genotype as GLV-1h68, with the exception that, in the expression cassette inserted into the TK gene, the DNA encoding the rat transferrin receptor is in the correct orientation for transcription from the vaccinia synthetic early/late promoter P_{SEL}. GLV-1h22 was constructed using the same method as used to create GLV-1h68, which is described in detail in U.S. Patent Publication No. 2005/0031643, with exception that the expression cassette inserted into the TK locus was generated using the pSC65-TfR transfer vector (also described in U.S. Patent Publication No. 2005/0031643; the parent vector for GLV-1h22 is RVGL19, which is shown in FIG. 1B and described in Example 1 of U.S. Patent Publi

Insertion of the expression cassettes into the LIVP genome in the generation of strains GLV-1h68 and GLV-1h22 resulted in disruption of the coding sequences for each of the F14.5L, TK and HA genes; accordingly, all three genes in the resulting strains are nonfunctional in that they do not encode the corresponding full-length proteins. As described in U.S. Patent Publication No. 2005/0031643, disruption of these genes not only attenuates the virus but also enhances its tumor-specific accumulation. Previous data have shown that systemic delivery of the GLV-1h68 virus in a mouse model of breast cancer resulted in the complete eradication of large subcutaneous GI-101A human breast carcinoma xenograft tumors in nude mice (see U.S. Patent Publication No. 2005/0031643).cation No. 2005/0031643).

The expression of the Ruc-GFP fusion protein by the recombinant virus was confirmed by luminescence assay and fluorescence microscopy. Expression of β-galactosidase and β-glucuronidase were confirmed by blue plaque formation upon addition of 5-bromo-4-chloro-3-indolyl-β-D-galactopyranoside (X-gal, Stratagene, La Jolla, Calif.) and 5-bromo-4-chloro-3-indolyl-β-D-glucuronic acid (X-GlcA, Research Product International Corporation, Mt. Prospect, Ill.), respectively. Positive plaques formed by recombinant virus were isolated and purified. The presence of expression cassettes in the F14.5L, TK and HA loci were also confirmed by PCR and DNA sequencing.

High titer viral preparations were obtained by centrifugation of viral precipitates in sucrose gradients (Joklik W K (1962) Virol. 18:9-18). For testing infection, CV-1 (1×10⁵) and GI-101A (4×10⁵) cells (Dr. A. Aller, Rumbaugh-Goodwin Institute for Cancer Research, Inc., Plantation, Fla.) were seeded onto 24-well plates. After 24 hours in culture, the cells were infected with individual viruses at a multiplicity of infection (MOI) of 0.001. The cells were incubated at 37° C. for 1 hour with brief agitation every 10 minutes to allow infection to occur. The infection medium was removed, and cells were incubated in fresh growth medium until cell harvest at 24, 48, 72, or 96 hours after infection. Viral particles from the infected cells were released by a quick freeze-thaw cycle, and the titers determined as pfu/ml of medium in duplicate by plaque assay in CV-1 cell monolayers. The same procedure was followed using a resting CV-1 cell culture, which was obtained by culturing a confluent monolayer of CV-1 cells for 6 days in DMEM supplemented with 5% FBS, before viral infection.

2. Construction of GLV-1h68 and GLV-1h22 Derivative Strains

Modified vaccinia viruses were generated by replacing nucleic acid or inserting nucleic acid at several loci in the GLV-1h68 vaccinia virus genome as follows: the F14.5L (also referred to as F3; see U.S. Patent Publication No. 2005/0031643), thymidine kinase (TK), hemagglutinin (HA) and A34R gene loci (the A34R gene encodes a C-type lectin-like glycoprotein, gp22-24, that is present in the outer membrane of extracellular enveloped virus (EEV), and that is reported to be required for infectivity of EEV; see, e.g., McIntosh et al. (1996) J. Virol. 70:272081). The heterologous DNA inserted either was (1) a relatively short non-coding DNA fragment, (2) an expression cassette containing protein-encoding DNA operably linked in the correct or reverse orientation to a vaccinia virus promoter, or (3) the coding sequence of the A34R gene (SEQ ID NO: 58) from vaccinia virus strain IHD-J.

Modified recombinant vaccinia viruses containing heterologous DNA inserted into one or more loci of the vaccinia virus genome were generated via homologous recombination between DNA sequences in the genome and a transfer vector using methods described herein and known to those of skill in the art (see, e.g., Falkner and Moss (1990) J. Virol. 64:3108-2111; Chakrabarti et al. (1985) Mol. Cell Biol. 5:3403-3409; and U.S. Pat. No. 4,722,848). In these methods, the existing target gene in the starting vaccinia virus genome is replaced by an interrupted copy of the gene contained in the transfer vector through two crossover events: a first crossover event of homologous recombination between the vaccinia virus genome and the transfer vector and a second crossover event of homologous recombination between direct repeats within the target locus. The interrupted version of the target gene that is in the transfer vector contains the insertion DNA flanked on each side by DNA corresponding to the left portion of the target gene and right portion of the target gene, respectively. The transfer vector also contains a dominant selection marker, e.g., the E. coli guanine phosphoribosyltransferase (gpt) gene, under the control of a vaccinia virus early promoter (e.g., P_{7.5kE}). Including such a marker in the vector enables a transient dominant selection process to identify recombinant virus grown under selective pressure that has incorporated the transfer vector within its genome. Because the marker gene is not stably integrated into the genome, it is deleted from the genome in a second crossover event that occurs when selection is removed. Thus, the final recombinant virus contains the interrupted version of the target gene as a disruption of the target loci, but does not retain the selectable marker from the transfer vector.

Homologous recombination between a transfer vector and a starting vaccinia virus genome occurred upon introduction of the transfer vector into cells that have been infected with the starting vaccinia virus. A series of transfer vectors was constructed as described below and the following modified vaccinia strains were constructed: GLV-1i69, GLV-1h70, GLV-1h71, GLV-1h72, GLV-1h73, GLV-1h74, GLV-1h81, GLV-1h82, GLV-1h83, GLV-1h84, GLV-1h85, GLV-1h86, GLV-1j87, GLV-1j88, GLV-1j89, GLV-1h90, GLV-1h91, GLV-1h92, GLV-1h96, GLV-1h97, GLV-1h98, GLV-1h100, GLV-1h101, GLV-1h139, GLV-1h104, GLV-1h105, GLV-1h106, GLV-1h107, GLV-1h108, GLV-1h109, GLV-1h146, GLV-1h150, GLV-1h151, GLV-1h152 and GLV-1h153. The construction of these strains is summarized in the following Table, which lists the modified vaccinia virus strains, including the previously described GLV-1h68, their respective genotypes, and the transfer vectors used to engineer the viruses:

TABLE 2
Generation of engineered vaccinia viruses

| Name of Virus | Parental Virus | VV Transfer Vector | Genotype |
|---|---|---|---|
| GLV-1h68 | — | — | F14.5L: (P_{SEL})Ruc-GFP |
| | | | TK: (P_{SEL})rTrfR-(P_{7.5k})LacZ |
| | | | HA: (P₁₁ₖ)gusA |
| GLV-1i69 | GLV-1h68 | A34R gene from | F14.5L: (P_{SEL})Ruc-GFP |
| | | VV IHD-J | TK: (P_{SEL})rTrfR-(P_{7.5k})LacZ |
| | | | HA: (P₁₁ₖ)gusA |
| | | | A34R: A34R-IHD-J |
| GLV-1h70 | GLV-1h68 | pNCVVhaT | F14.5L: (P_{SEL})Ruc-GFP |
| | | | TK: (P_{SEL})rTrfR-(P_{7.5k})LacZ |
| | | | HA: HindIII-BamHI |
| GLV-1h71 | GLV-1h68 | pNCVVf14.5lT | F14.5L: BamHI-HindIII |
| | | | TK: (P_{SEL})rTrfR-(P_{7.5k})LacZ |
| | | | HA: (P₁₁ₖ)gusA |
| GLV-1h72 | GLV-1h68 | pCR-TKLR-gpt2 | F14.5L: (P_{SEL})Ruc-GFP |
| | | | TK: SacI-BamHI |
| | | | HA: (P₁₁ₖ)gusA |
| GLV-1h73 | GLV-1h70 | pNCVVf14.5lT | F14.5L: BamHI-HindIII |
| | | | TK: (P_{SEL})rTrfR-(P_{7.5k})LacZ |
| | | | HA: HindIII-BamHI |
| GLV-1h74 | GLV-1h73 | pCR-TKLR-gpt2 | F14.5L: BamHI-Hind III |
| | | | TK: SacI-BamHI |
| | | | HA: HindIII-BamHI |
| GLV-1h81 | GLV-1h68 | pNCVVhaT-SEL-hk5 | F14.5L: (P_{SEL})Ruc-GFP |
| | | | TK: (P_{SEL})rTrfR-(P_{7.5k})LacZ |
| | | | HA: (P_{SEL})hk-5 |
| GLV-1h82 | GLV-1h22 | pNCVVhaT-ftn | F14.5L: (P_{SEL})Ruc-GFP |
| | | | TK: (P_{SEL})TrfR-(P_{7.5k})LacZ |
| | | | HA: (P_{SEL})ftn |
| GLV-1h83 | GLV-1h68 | pNCVVhaT-ftn | F14.5L: (P_{SEL})Ruc-GFP |
| | | | TK: (P_{SEL})rTrfR-(P_{7.5k})LacZ |
| | | | HA: (P_{SEL})ftn |
| GLV-1h84 | GLV-1h73 | pCR-TK-SEL-mRFP1 | F14.5L: BamHI-Hind III |
| | | | TK: (P_{SEL})CBG99-mRFP1 |
| | | | HA: Hind III-BamHI |
| GLV-1h85 | GLV-1h72 | pNCVVf14.5lT | F14.5L: BamHI-HindIII |
| | | | TK: Sac I-BamHI |
| | | | HA: (P₁₁ₖ)gusA |
| GLV-1h86 | GLV-1h72 | pNCVVhaT | F14.5L: (P_{SEL})Ruc-GFP |
| | | | TK: Sac I-BamHI |
| | | | HA: Hind III-BamHI |
| GLV-1j87 | GLV-1h68 | pCR-gpt-dA35R6 | F14.5L: (P_{SEL})Ruc-GFP |
| | | | TK: (P_{SEL})rTrfR-(P_{7.5k})LacZ |
| | | | HA: (P₁₁ₖ)gusA |
| | | | A35R: Multiple cloning |
| | | | sites (MCS) |
| GLV-1j88 | GLV-1h73 | pCR-gpt-dA35R6 | F14.5L: BamHI-HindIII |
| | | | TK: (P_{SEL})rTrfR-(P_{7.5k})LacZ |
| | | | HA: HindIII-BamHI |
| | | | A35R: MCS |
| GLV-1j89 | GLV-1h74 | pCR-gpt-dA35R6 | F14.5L: BamHI-HindIII |
| | | | TK: SacI-BamHI |
| | | | HA: HindIII-BamHI |
| | | | A35R: MCS |
| GLV-1h90 | GLV-1h68 | HA-SE-IL-6-1 | F14.5L: (P_{SEL})Ruc-GFP |
| | | | TK: (P_{SEL})rTrfR-(P_{7.5k})LacZ |
| | | | HA: (P_{SE})sIL-6R/IL-6 |
| GLV-1h91 | GLV-1h68 | HA-SEL-IL-6-1 | F14.5L: (P_{SEL})Ruc-GFP |
| | | | TK: (P_{SEL})rTrfR-(P_{7.5k})LacZ |
| | | | HA: (P_{SEL})sIL-6R/IL-6 |
| GLV-1h92 | GLV-1h68 | HA-SL-IL-6-1 | F14.5L: (P_{SEL})Ruc-GFP |
| | | | TK: (P_{SEL})rTrfR-(P_{7.5k})LacZ |
| | | | HA: (P_{SL})sIL-6R/IL-6 |
| GLV-1h96 | GLV-1h68 | FSE-IL-24 | F14.5L: (P_{SE})IL-24 |
| | | | TK: (P_{SEL})rTrfR-(P_{7.5k})LacZ |
| | | | HA: (P₁₁ₖ)gusA |
| GLV-1h97 | GLV-1h68 | FSEL-IL-24 | F14.5L: (P_{SEL})IL-24 |
| | | | TK: (P_{SEL})rTrfR-(P_{7.5k})LacZ |
| | | | HA: (P₁₁ₖ)gusA |
| GLV-1h98 | GLV-1h68 | FSL-IL-24 | F14.5L: (P_{SL})IL-24 |
| | | | TK: (P_{SEL})rTrfR-(P_{7.5k})LacZ |
| | | | HA: (P₁₁ₖ)gusA |
| GLV-1h104 | GLV-1h68 | pCR-TK-SE-tTF-RGD | F14.5L: (P_{SEL})Ruc-GFP |
| | | | TK: (P_{SE})tTF-RGD |
| | | | HA: (P₁₁ₖ)gusA |
| GLV-1h105 | GLV-1h68 | pCR-TK-SEL-tTF-RGD | F14.5L: (P_{SEL})Ruc-GFP |
| | | | TK: (P_{SEL})tTF-RGD |
| | | | HA: (P₁₁ₖ)gusA |
| GLV-1h106 | GLV-1h68 | pCR-TK-SL-tTF-RGD | F14.5L: (P_{SEL})Ruc-GFP |
| | | | TK: (P_{SL})tTF-RGD |
| | | | HA: (P₁₁ₖ)gusA |
| GLV-1h107 | GLV-1h68 | pCR-TK-SE-G6-FLAG | F14.5L: (P_{SEL})Ruc-GFP |
| | | | TK: (P_{SE})G6-FLAG |
| | | | HA: (P₁₁ₖ)gusA |
| GLV-1h108 | GLV-1h68 | pCR-TK-SEL-G6-FLAG | F14.5L: (P_{SEL})Ruc-GFP |
| | | | TK: (P_{SEL})G6-FLAG |
| | | | HA: (P₁₁ₖ)gusA |
| GLV-1h109 | GLV-1h68 | pCR-TK-SL- G6-FLAG | F14.5L: (P_{SEL})Ruc-GFP |
| | | | TK: (P_{SL})G6-FLAG |
| | | | HA:(P₁₁ₖ)gusA |
| GLV-1h99 | GLV-1h68 | FSE-hNET | F14.5L: (PSE)hNET |
| | | | TK: (PSEL)rTrfR-(P7.5k)LacZ |
| | | | HA: (P11k)gusA |
| GLV-1h100 | GLV-1h68 | TK-SE-hNET3 | F14.5L: (PSEL)Ruc-GFP |
| | | | TK: (PSE)hNET |
| | | | HA: (P11k)gusA |
| GLV-1h101 | GLV-1h68 | TK-SL-hNET3 | F14.5L: (PSEL)Ruc-GFP |
| | | | TK: (PSL)hNET |
| | | | HA: (P11k)gusA |
| GLV-1h139 | GLV-1h68 | HA-SE-hNET-1 | F14.5L: (PSEL)Ruc-GFP |
| | | | TK: (PSEL)rTrfR-(P7.5k)LacZ |
| | | | HA: (PSE)hNET |
| GLV-1h146 | GLV-1h100 | HA-SE-IL-24-1 | F14.5L: (PSEL)Ruc-GFP |
| | | | TK: (PSE)hNET |
| | | | HA: (PSE)IL-24 |
| GLV-1h150 | GLV-1h101 | HA-SE-IL-24-1 | F14.5L: (PSEL)Ruc-GFP |
| | | | TK: (PSL)hNET |
| | | | HA: (PSE)IL-24 |
| GLV-1h151 | GLV-1h68 | HA-SE-hNIS-1 | F14.5L: (PSEL)Ruc-GFP |
| | | | TK: (PSEL)rTrfR-(P7.5k)LacZ |
| | | | HA: (PSE)hNIS |
| GLV-1h152 | GLV-1h68 | HA-SEL-hNIS-2 | F14.5L: (PSEL)Ruc-GFP |
| | | | TK: (PSEL)rTrfR-(P7.5k)LacZ |
| | | | HA: (PSEL)hNIS |
| GLV-1h153 | GLV-1h68 | HA-SL-hNIS-1 | F14.5L: (PSEL)Ruc-GFP |
| | | | TK: (PSEL)rTrfR-(P7.5k)LacZ |
| | | | HA: (PSL)hNIS |

Briefly, the strains listed in the table were generated as follows (further details are provided below):

GLV-1i69 was generated by replacement of the coding sequence of the A34R gene in starting strain GLV-1h68 (nucleotides 153693 to 154199 in SEQ ID NO: 1) with the A34R gene from well-known vaccinia virus IHD-J strain.

GLV-1h70 was generated by insertion of a short non-coding DNA fragment containing HindIII and BamHI sites into the HA locus of starting strain GLV-1h68 thereby deleting the gusA expression cassette at the HA locus of GLV-1h68. Thus, in strain GLV-1h70, the vaccinia HA gene is interrupted within the coding sequence by a short non-coding DNA fragment.

GLV-1h71 was generated by insertion of a short non-coding DNA fragment containing BamHI and HindIII sites (SEQ ID NO: 12) into the F14.5L locus of starting strain GLV-1h68 thereby deleting the Ruc-GFP fusion gene expression cassette at the F14.5L locus of GLV-1h68. Thus, in strain GLV-1h71, the vaccinia F14.5L gene is interrupted within the coding sequence by a short non-coding DNA fragment.

GLV-1h72 was generated by insertion of a short non-coding DNA fragment containing SacI and BamHI sites (SEQ ID NO: 18) into the TK locus of starting strain GLV-1h68 thereby deleting the LacZ/rTFr expression cassette at the TK locus in GLV-1h68. Thus, in strain GLV-1h72, the vaccinia TK gene is interrupted within the coding sequence by a short non-coding DNA fragment.

GLV-1h73 was generated by insertion of a short non-coding DNA fragment containing BamHI and HindIII sites (SEQ ID NO: 12) into the F14.5L locus of GLV-1h70 thereby deleting the Ruc-GFP fusion gene expression cassette at the F14.5L locus of GLV-1h70. Thus, in strain GLV-1h73, the vaccinia HA and F14.5L genes are interrupted within the coding sequence by a short non-coding DNA fragment.

GLV-1h74 was generated by insertion of a short non-coding DNA fragment containing Sad and BamHI sites (SEQ ID NO: 18) into the TK locus of strain GLV-1h73 thereby deleting the LacZ/rTFr expression cassette at the TK locus of GLV-1h73. Thus, in strain GLV-1h74, the vaccinia HA, F14.5L and TK genes are interrupted within the coding sequence by a short non-coding DNA fragment.

GLV-1h81 was generated by insertion of an expression cassette encoding the plasminogen K5 domain under the control of the vaccinia P_{SEL} promoter into the HA locus of starting strain GLV-1h68 thereby deleting the gusA expression cassette at the HA locus of starting GLV-1h68. Thus, in strain GLV-1h81, the vaccinia HA gene is interrupted within the coding sequence by a DNA fragment containing DNA encoding the plasminogen K5 domain operably linked to the vaccinia synthetic early/late promoter.

GLV-1h82 was generated by insertion of an expression cassette encoding E. coli ferritin under the control of the vaccinia P_{SEL} promoter into the HA locus of strain GLV-1h22 thereby deleting the gusA expression cassette at the HA locus of GLV-1h22. Thus, in strain GLV-1h82, the vaccinia HA gene is interrupted within the coding sequence by a DNA fragment containing DNA encoding E. coli ferritin operably linked to the vaccinia synthetic early/late promoter

GLV-1h83 was generated by insertion of an expression cassette encoding E. coli ferritin under the control of the vaccinia P_{SEL} promoter into the HA locus of starting strain GLV-1h68 thereby deleting the gusA expression cassette at the HA locus of GLV-1h68. Thus, in strain GLV-1h83, the vaccinia HA gene is interrupted within the coding sequence by a DNA fragment containing DNA encoding E. coli ferritin operably linked to the vaccinia synthetic early/late promoter.

GLV-1h84 was generated by insertion of an expression cassette containing

DNA encoding CBG99 and mRFP1 connected through a picornavirus 2A element and under the control of the vaccinia synthetic early/late promoter (P_{SEL}) into the TK locus of strain GLV-1h73 thereby deleting the LacZ/rTFr expression cassette at the TK locus of GLV-1h73. Thus, in strain GLV-1h84, the vaccinia HA and F14.5L genes are interrupted within the coding sequence by a short non-coding DNA fragment, and the vaccinia TK gene is interrupted within the coding sequence by DNA encoding a fusion of CBG99 and mRFP1 proteins. Since DNAs encoding both marker proteins (CBG99 and mRFP1) are under the control of the same promoter, only one transcript is produced. During translation, these two proteins are cleaved into two individual proteins at picornavirus 2A element (Osborn et al., Mol. Ther. 12: 569-74, 2005). CBG99 produces a more stable luminescent signal than does Renilla luciferase with a half-life of greater than 30 minutes, which makes both in vitro and in vivo assays more convenient. mRFP1 provides improvements in in vivo imaging relative to GFP since mRFP1 can penetrate tissue deeper than GFP.

GLV-1h85 was generated by insertion of a short non-coding DNA fragment containing BamHI and HindIII sites into the F14.5L locus of strain GLV-1h72 thereby deleting the Ruc-GFP fusion gene expression cassette at the F14.5L locus of GLV-1h72. Thus, in strain GLV-1h85, the vaccinia F14.5L and TK genes are interrupted within the coding sequence by a short non-coding DNA fragment.

GLV-1h86 was generated by insertion of a short non-coding DNA fragment containing HindIII and BamHI sites into the HA locus of strain GLV-1h72 thereby deleting the gusA expression cassette at the HA locus of GLV-1h72. Thus, in strain

GLV-1h86, the vaccinia TK and HA genes are interrupted within the coding sequence by a short non-coding DNA fragment

GLV-1j87 was generated by deletion the coding sequence of the A35R gene in starting strain GLV-1h68 (nucleotides 154,243 to 154,773 in SEQ ID NO: 1). Thus, in strain GLV-1j87, the vaccinia A35 gene is replaced by a short non-coding DNA fragment.

GLV-1j88 was generated by deletion the coding sequence of the A35R gene in starting strain GLV-1h73. Thus, in strain GLV-1j88, the vaccinia A35 gene is replaced by a short non-coding DNA fragment.

GLV-1j89 was generated by deletion the coding sequence of the A35R gene in starting strain GLV-1h74. Thus, in strain GLV-1j89, the vaccinia A35 gene is replaced by a short non-coding DNA fragment.

GLV-1h90 was generated by insertion of an expression cassette encoding human IL-6 fused to the 3′ end of the cDNA encoding human soluble IL-6 receptor (sIL-6R, aa 1-323) under the control of the vaccinia P_{SE} promoter into the HA locus of starting strain GLV-1h68, thereby deleting the gusA expression cassette at the HA locus of starting GLV-1h68. Thus, in strain GLV-1h90, the vaccinia HA gene is interrupted within the coding sequence by a DNA fragment containing DNA encoding human IL-6 fused to the 3′ end of the cDNA encoding human soluble IL-6 receptor operably linked to the vaccinia synthetic early promoter.

GLV-1h91 was generated by insertion of an expression cassette encoding sIL-6R under the control of the vaccinia P_{SEL} promoter into the HA locus of starting strain GLV-1h68, thereby deleting the gusA expression cassette at the HA locus of starting GLV-1h68. Thus, in strain GLV-1h91, the vaccinia HA gene is interrupted within the coding sequence by a DNA fragment containing DNA encoding human IL-6 fused to the 3′ end of the cDNA encoding human soluble IL-6 receptor operably linked to the vaccinia synthetic early/late promoter.

GLV-1h92 was generated by insertion of an expression cassette encoding sIL-6R under the control of the vaccinia P_{SL} promoter into the HA locus of starting strain GLV-1h68, thereby deleting the gusA expression cassette at the HA locus of starting GLV-1h68. Thus, in strain GLV-1h92, the vaccinia HA gene is interrupted within the coding sequence by a DNA fragment containing DNA encoding human IL-6 fused to the 3′ end of the cDNA encoding human soluble IL-6 receptor operably linked to the vaccinia synthetic late promoter.

GLV-1h96 was generated by insertion of an expression cassette encoding the IL-24 under the control of the vaccinia P_{SE} promoter into the F14.5L locus of starting strain GLV-1h68, thereby deleting the Ruc-GFP fusion gene expression cassette at the F14.5L locus of GLV-1h68. Thus, in strain GLV-1h96, the vaccinia F14.5L gene is interrupted within the coding sequence by a DNA fragment containing DNA encoding IL-24 operably linked to the vaccinia synthetic early promoter.

GLV-1h97 was generated by insertion of an expression cassette encoding IL-24 under the control of the vaccinia P_{SEL} promoter into the F14.5L locus of starting strain GLV-1h68, thereby deleting the Ruc-GFP fusion gene expression cassette at the F14.5L locus of GLV-1h68. Thus, in strain GLV-1h97, the vaccinia F14.5L gene is interrupted within the coding sequence by a DNA fragment containing DNA encoding FCU operably linked to the vaccinia synthetic early/late promoter.

GLV-1h98 was generated by insertion of an expression cassette encoding IL-24 under the control of the vaccinia P_{SL} promoter into the F14.5L locus of starting strain GLV-1h68, thereby deleting the Ruc-GFP fusion gene expression cassette at the F14.5L locus of GLV-1h68. Thus, in strain GLV-1h98, the vaccinia F14.5L gene is interrupted within the coding sequence by a DNA fragment containing DNA encoding IL-24 operably linked to the vaccinia synthetic late promoter.

GLV-1h104 was generated by insertion of an expression cassette containing DNA encoding truncated human tissue factor fused to the αᵥβ₃-integrin RGD binding motif (tTF-RGD) under the control of the vaccinia synthetic early promoter (P_{SE}) into the TK locus of strain GLV-1h68 thereby deleting the LacZ/rTFr expression cassette at the TK locus of GLV-1h68. Strain GLV-1h104 retains the Ruc-GFP expression cassette at the F14.5L locus and the gusA expression cassette at the HA locus.

GLV-1h105 was generated by insertion of an expression cassette containing DNA encoding tTF-RGD fusion protein under the control of the vaccinia synthetic early/late promoter (P_{SEL}) into the TK locus of strain GLV-1h68 thereby deleting the LacZ/rTFr expression cassette at the TK locus of GLV-1h68. Strain GLV-1h105 retains the Ruc-GFP expression cassette at the F14.5L locus and the gusA expression cassette at the HA locus.

GLV-1h106 was generated by insertion of an expression cassette containing DNA encoding tTF-RGD fusion protein under the control of the vaccinia synthetic late promoter (P_{SL}) into the TK locus of strain GLV-1h68 thereby deleting the LacZ/rTFr expression cassette at the TK locus of GLV-1h68. Strain GLV-1h106 retains the Ruc-GFP expression cassette at the F14.5L locus and the gusA expression cassette at the HA locus.

GLV-1h107 was generated by insertion of an expression cassette containing DNA encoding scFv anti-VEGF-FLAG fusion protein (G6-FLAG) under the control of the vaccinia synthetic early promoter (P_{SE}) into the TK locus of strain GLV-1h68 thereby deleting the LacZ/rTFr expression cassette at the TK locus of GLV-1h68. Strain GLV-1h107 retains the Ruc-GFP expression cassette at the F14.5L locus and the gusA expression cassette at the HA locus.

GLV-1h108 was generated by insertion of an expression cassette containing DNA encoding G6-FLAG fusion protein under the control of the vaccinia synthetic early/late promoter (P_{SEL}) into the TK locus of strain GLV-1h68 thereby deleting the LacZ/rTFr expression cassette at the TK locus of GLV-1h68. Strain GLV-1h108 retains the Ruc-GFP expression cassette at the F14.5L locus and the gusA expression cassette at the HA locus.

GLV-1h109 was generated by insertion of an expression cassette containing DNA encoding G6-FLAG fusion protein under the control of the vaccinia synthetic late promoter (P_{SL}) into the TK locus of strain GLV-1h68 thereby deleting the LacZ/rTFr expression cassette at the TK locus of GLV-1h68. Strain GLV-1h109 retains the Ruc-GFP expression cassette at the F14.5L locus and the gusA expression cassette at the HA locus.

GLV-1h99 was generated by insertion of an expression cassette encoding hNET under the control of the vaccinia P_{SE} promoter into the F14.5L locus of starting strain GLV-1h68, thereby deleting the Ruc-GFP fusion gene expression cassette at the F14.5L locus of starting GLV-1h68. Thus, in strain GLV-1h99, the vaccinia F14.5L gene is interrupted within the coding sequence by a DNA fragment containing DNA encoding hNET operably linked to the vaccinia synthetic early promoter.

GLV-1h100 was generated by insertion of an expression cassette encoding hNET under the control of the vaccinia P_{SE} promoter into the TK locus of starting strain GLV-1h68 thereby deleting the LacZ/rTFr expression cassette at the TK locus of starting GLV-1h68. Thus, in strain GLV-1h100, the vaccinia TK gene is interrupted within the coding sequence by a DNA fragment containing DNA encoding hNET operably linked to the vaccinia synthetic early promoter.

GLV-1h101 was generated by insertion of an expression cassette encoding hNET under the control of the vaccinia P_{SL} promoter into the TK locus of starting strain GLV-1h68 thereby deleting the LacZ/rTFr expression cassette at the TK locus of starting GLV-1h68. Thus, in strain GLV-1h101, the vaccinia TK gene is interrupted within the coding sequence by a DNA fragment containing DNA encoding hNET operably linked to the vaccinia synthetic late promoter.

GLV-1h139 was generated by insertion of an expression cassette encoding hNET under the control of the vaccinia P_{SE} promoter into the HA locus of starting strain GLV-1h68 thereby deleting the gusA expression cassette at the HA locus of starting GLV-1h68. Thus, in strain GLV-1h139, the vaccinia HA gene is interrupted within the coding sequence by a DNA fragment containing DNA encoding hNET operably linked to the vaccinia synthetic early promoter.

GLV-1h146 was generated by insertion of an expression cassette encoding IL-24 under the control of the vaccinia P_{SE} promoter into the HA locus of starting strain GLV-1h100, thereby deleting the gusA expression cassette at the HA locus of starting GLV-1h100. Thus, in strain GLV-1h146, the vaccinia HA gene is interrupted within the coding sequence by a DNA fragment containing DNA encoding IL-24 operably linked to the vaccinia synthetic early promoter and the vaccinia TK gene is interrupted within the coding sequence by a DNA fragment containing DNA encoding hNET operably linked to the vaccinia synthetic early promoter.

GLV-1h150 was generated by insertion of an expression cassette encoding IL-24 under the control of the vaccinia P_{SE} promoter into the HA locus of starting strain GLV-1h101, thereby deleting the gusA expression cassette at the HA locus of starting GLV-1h101. Thus, in strain GLV-1h150, the vaccinia HA gene is interrupted within the coding sequence by a DNA fragment containing DNA encoding IL-24 operably linked to the vaccinia synthetic early promoter and the vaccinia TK gene is interrupted within the coding sequence by a DNA fragment containing DNA encoding hNET operably linked to the vaccinia synthetic late promoter.

GLV-1h151 was generated by insertion of an expression cassette encoding hNIS under the control of the vaccinia P_{SE} promoter into the HA locus of starting strain GLV-1h68 thereby deleting the gusA expression cassette at the HA locus of starting GLV-1h68. Thus, in strain GLV-1h151, the vaccinia HA gene is interrupted within the coding sequence by a DNA fragment containing DNA encoding hNIS operably linked to the vaccinia synthetic early promoter.

GLV-1h152 was generated by insertion of an expression cassette encoding hNIS under the control of the vaccinia P_{SEL} promoter into the HA locus of starting strain GLV-1h68 thereby deleting the gusA expression cassette at the HA locus of starting GLV-1h68. Thus, in strain GLV-1h152, the vaccinia HA gene is interrupted within the coding sequence by a DNA fragment containing DNA encoding hNIS operably linked to the vaccinia synthetic early/late promoter.

GLV-1h153 was generated by insertion of an expression cassette encoding hNIS under the control of the vaccinia P_{SL} promoter into the HA locus of starting strain GLV-1h68 thereby deleting the gusA expression cassette at the HA locus of starting GLV-1h68. Thus, in strain GLV-1h153, the vaccinia HA gene is interrupted within the coding sequence by a DNA fragment containing DNA encoding hNIS operably linked to the vaccinia synthetic late promoter.

2. VV Transfer Vectors Employed for the Production of Modified Vaccinia Viruses

The following vectors were constructed and employed as described below to generate the recombinant vaccinia viral strains.

a. pNCVVhaT: For Insertion of Non-Coding Heterologous DNA into the Vaccinia Virus HA Locus

The pNCVVhaT vector (SEQ ID NO: 4) was employed to create vaccinia virus strains GLV-1h70 and GLV-1h86 having the following genotypes: F14.5L: (P_{SEL})Ruc-GFP, TK: (P_{SEL})rTrfR-(P_{7.5k})LacZ (strain GLV-1h70), HA: HindIII-BamHI and F14.5L: (P_{SEL})Ruc-GFP, TK: SacI-BamHI, HA: HindIII-BamHI (strain GLV-1h86). Strains GLV-1h70 and GLV-1h86 were generated by inserting a short non-coding DNA fragment containing HindIII and BamHI sites (SEQ ID NO: 5; taagcttcgcaggatccc) into the HA locus of strains GLV-1h68 and GLV-1h72, respectively, thereby deleting the gusA expression cassette at the hemagglutinin (HA) locus of GLV-1h68 and GLV-1h72. Vector pNCVVhaT contains the non-coding DNA fragment flanked by sequences of the HA gene, the E. coli guanine phosphoribosyltransferase (gpt) gene under the control of the vaccinia virus P_{7.5kE} promoter for transient dominant selection of virus that has incorporated the vector, and sequences of the pUC plasmid. The left and right flanking sequences of the VV HA gene (also named A56R, see nucleotides 161420 to 162352 of SEQ ID NO: 2) that were incorporated into the vector correspond to nucleotides 161423 to 161923 and nucleotides 162037 to 162394, respectively of SEQ ID NO: 2. The HA gene flanking DNAs were PCR-amplified from VV LIVP using Platinum PCR SuperMix High Fidelity (Invitrogen, Carlsbad, Calif.) and the following primers containing the non-coding DNA sequence:

Left flank:
(SEQ ID NO: 6)
5′-GCGCATATGACACGATTACCAATACTTTTG-3′
and
(SEQ ID NO: 7)
5′-GTCGGGATCCTGCGAAGCTTAGATTTCGAATACCGACGAGC-3′,
Right Flank:
(SEQ ID NO: 8)
5′-GAAATCTAAGCTTCGCAGGATCCCGACTCCGGAACCAATTACTG-3′
and
(SEQ ID NO: 9)
5′-GCGGAATTCTGATAGATTTTACTATCCCAG-3′.

The two fragments were joined using the method of gene-splicing by overlapping extension (see, e.g., Horton et al., Methods Enzymol., 217:270-279 (1993)). The resulting fragment was digested with NdeI and EcoRI and cloned into the same-cut vector pUCP7.5-gpt-1 (SEQ ID NO: 10) to generate the construct pNCVVhaT. The flanking sequences of HA in the target vector were confirmed by sequencing and were identical to nucleotides 161423 to 161923 of SEQ ID NO: 2 (left flank) and nucleotides 162037 to 162394 of SEQ ID NO: 2 (right flank).

b. pNCVVf14.51T: for Insertion of Non-Coding Heterologous DNA into the Vaccinia F14.5L Locus

The pNCVVf14.51T vector (SEQ ID NO: 11) was employed to create vaccinia virus strains GLV-1h71, GLV-1h73 and GLV-1h85 having the following genotypes: F14.5L: BamHI-HindIII, TK: (P_{SEL})rTrfR-(P_{7.5k})LacZ (GLV-1h71), HA: (P₁₁ₖ)_{g}usA; F14.5L: BamHI-HindIII, TK: (P_{SEL})ᵣTrfR-(P_{7.5k})LacZ (GLV-1h73), HA:HindIII-BamHI and F14.5L: BamHI-HindIII, TK:SacI-BamHI (GLV-1h85), HA: (P₁₁ₖ)gusA. Strains GLV-1h71, GLV-1h73 and GLV-1h85 were generated by inserting a short non-coding DNA fragment containing Bam HI and HindIII sites (SEQ ID NO: 12; aggatcctgcgaagct) into the F14.5L locus of strains GLV-1h68, GLV-1h70 and GLV-1h72, respectively, thereby deleting the Ruc-GFP fusion gene expression cassette at the F14.5L locus of these strains. Vector pNCVVf14.51T contains the non-coding DNA fragment flanked by sequences of the F14.5L gene, the E. coli guanine phosphoribosyltransferase (gpt) gene under the control of the vaccinia virus P_{7.5kE} promoter for transient dominant selection of virus that has incorporated the vector, and sequences of the pUC plasmid. The left and right flanking sequences of the VV F14.5L gene (see nucleotides 41476 to 41625 of SEQ ID NO: 2) that were incorporated into the vector correspond to nucleotides 41593 to 42125 and nucleotides 41018 to 41592, respectively of SEQ ID NO: 2. The F14.5L gene flanking DNAs were PCR-amplified from VV LIVP using Platinum PCR SuperMix High Fidelity and the following primers containing the non-coding DNA sequence:

Left Flank:
(SEQ ID NO: 13)
5′-GCGCATATGTAGAAGAATTGATAAATATG-3′
and
(SEQ ID NO: 14)
5′-GCCGCAGGATCCTGCGAAGCTTACAGACACGAATATGACTAAACCGA
TG-3′,
Right Flank:
(SEQ ID NO: 15)
5′-GTCTGTAAGCTTCGCAGGATCCTGCGGCCGCCATCGTCGGTGTGTTG
TC-3′
and
(SEQ ID NO: 16)
5′-GCGGAATTCAGAGGATTACAACAAAAAGATG-3′.

The two fragments were joined together as described above (gene-splicing by overlapping extension). The resulting fragment was digested with NdeI and EcoRI and cloned into the same-cut vector pUCP7.5-gpt-1 (SEQ ID NO: 10) to generate the construct pNCVVf14.51T (SEQ ID NO: 11). The flanking sequences of F14.5L in the target vector were confirmed by sequencing and were identical to nucleotides 41593 to 42125 of SEQ ID NO: 2 (left flank) and nucleotides 41018 to 41592 of SEQ ID NO: 2 (right flank).

c. pCR-TKLR-gpt2: for Insertion of Non-Coding Heterologous DNA in the Vaccinia TK Locus

The pCR-TKLR-gpt2 vector (SEQ ID NO: 17) was employed to create vaccinia virus strains GLV-1h72 and GLV-1h74 having the following genotypes: F14.5L: (P_{SEL})Ruc-GFP, TK: SacI-BamHI (GLV-1h72), HA: (P₁₁ₖ)gusA and F14.5L: BamHI-HindIII, TK: SacI-BamHI (GLV-1h74), HA:HindIII-BamHI. Strain GLV-1h72 was generated by inserting a short non-coding DNA fragment containing SacI and BamHI sites (SEQ ID NO: 18; ggtaccgagctcggatcc) into the TK locus of starting strain GLV-1h68 thereby deleting the LacZ/rTFr expression cassette at the TK locus of GLV-1h68. Strain GLV-1h74 was generated by inserting the short non-coding DNA fragment containing SacI and Bam HI sites into the TK locus of strain GLV-1h73 thereby deleting the LacZ/rTFr expression cassette at the TK locus of GLV-1 h73.

Vector pCR-TKLR-gpt2 was generated from vector pCR2.1 (Invitrogen, Carlsbad, Calif., SEQ ID NO: 21) and contains the non-coding DNA fragment flanked by sequences of the TK gene and the E. coli guanine phosphoribosyltransferase (gpt) gene under the control of the vaccinia virus P_{7.5kE} promoter for transient dominant selection of virus that has incorporated the vector. The left flank (TK_{L}) of the TK locus in the LIVP genome that was incorporated into the vector corresponds to nucleotides 79726 to 80231 of SEQ ID NO: 2 (TK locus in the LIVP genome is located at nucleotides 78142 to 80961 of SEQ ID NO: 2). The left flank DNA was PCR amplified with the primers TK_{L}-5 (5′-ATAAGCTTTGTTACAGATGGAAGGGTCAAA-3′, SEQ ID NO: 19) and TK_{L}-3 (5′-AGGTACCGTTTGCCATACGCTCACAGA-3′, SEQ ID NO: 20) using Invitrogen High Fidelity PCR mix. The PCR product was digested with HindIII and KpnI, and inserted into the corresponding sites in vector pCR2.1 (Invitrogen, Carlsbad, Calif., SEQ ID NO: 21), resulting in pCP-TKL1 (SEQ ID NO: 22). The right flanking region (TK_{R}) of the TK locus in the LIVP genome that was incorporated into the vector corresponds to nucleotides 80211 to 80730 of SEQ ID NO: 2. The right flank DNA was PCR amplified with the primers: TK_{R}-5 (5′-TGAGCTCGGATCCTTCTGTGAGCGTATGGCAAA-3′, SEQ ID NO: 23) and TK_{R}-3 (5′-TTACTAGTACACTACGGTGGCACCATCT-3′, SEQ ID NO: 24). The PCR product was digested with BamHI and SpeI and cloned into the corresponding sites in vector pCR2.1 to yield pCR-TKR4 (SEQ ID NO: 25). The pCR-TKL1 and pCR-TKR4 contained the correct sequences of TK_{L} and TK_{R}, respectively, as confirmed by sequencing and were identical to nucleotides 79726 to 80231 of SEQ ID NO: 2 (left flank) and nucleotides 80211 to 80730 of SEQ ID NO: 2 (right flank). The insert TK_{L} was then excised from pCR-TKL1 by restriction digestion with HindIII and BamHI and inserted into the same-cut vector pCR-TKR4 to yield pCR-TKLR1 (SEQ ID NO: 26) thereby joining the left and right flanking sequences with the non-coding DNA between them in a single fragment.

In order to add DNA encoding Escherichia coli guanine phosphoribosyltransferase (gpt) linked to the vaccinia virus promoter p7.5 k to pCR-TKLR1 for use in transient dominant selection, a DNA fragment containing these elements was amplified with the primers gpt5 (5′-TCCCAGTCACGACGTTGTAA-3′, SEQ ID NO: 27) and gpt3 (5′-TGATTACGCCAAGCTGATCC-3′, SEQ ID NO: 28) from pUCP7.5-gpt-1 and cloned into vector pCR2.1. The sequence of the insert p7.5 k-gpt was confirmed and released with EcoRI and cloned into the same-cut vector pCR-TKLR1 to generate the final transfer vector pCR-TKLR-gpt2 (SEQ ID NO: 17).

d. pNCVVhaT-SEL-hk5: for Insertion of an Expression Cassette Encoding Plasminogen Kringle 5 Domain Under the Control of the Vaccinia P_{SEL} Promoter into the Vaccinia HA Locus

Vector pNCVVhaT-SEL-hk5 (SEQ ID NO: 41) was employed to develop strain GLV-1h81 having the following genotype: F14.5L: (P_{SEL})Ruc-GFP, TK: (P_{SEL})rTrfT-(P_{7.5k})LacZ, HA: (P_{SEL})hk-5. Strain GLV-1h81 was generated by inserting DNA encoding the human plasminogen kringle 5 domain (SEQ ID NO: 42) operably linked to the vaccinia virus synthetic early/late promoter (P_{SEL}) (SEQ ID NO: 29) into the HA locus of starting strain GLV-1h68 thereby deleting the gusA expression cassette at the HA locus of GLV-1h68. Vector pNCVVhaT-SEL-hk5 contains a DNA fragment encoding the human plasminogen kringle 5 domain operably linked to the vaccinia synthetic early/late promoter (P_{SEL}), sequences of the HA gene flanking the (PsEL)hk-5 DNA fragment, the E. coli guanine phosphoribosyltransferase (gpt) gene under the control of the vaccinia virus P_{7.5kE} promoter for transient dominant selection of virus that has incorporated the vector, and sequences of the pUC plasmid.

To generate vector pNCVVhaT-SEL-hk5, DNA encoding human plasminogen kringle 5 was PCR-amplified from the plasmid pBLAST-hKringle5 (Invivogen, San Diego, Calif.; SEQ ID NO: 43) using AccuPrime Pfx SuperMix (Invitrogen, Carlsbad, Calif.) and primers: 5′-GCGAAGCTTACCATGTACAGGATGCAACTCCTGTCTTG-3′ (SEQ ID NO: 44) and 5′-GCGGGATCCAGAAAAACTAATCAAATGAAGGGGCCGCACACTG-3′ (SEQ ID NO: 45). The PCR product was digested with HindIII and BamHI and cloned into the same-cut vector pNCVVhaT-SEL-ADP-V5 (SEQ ID NO: 46); similar to pNCVVhaT, but contains ADP-V5 under the control of the synthetic early/late promoter in between the flanking sequences of HA to replace adenovirus death protein (ADP) gene tagged with V5 at 3′ end. The sequence of the human plasminogen kringle 5 domain was confirmed by sequencing.

e. pNCVVhaT-ftn: for Insertion of an Expression Cassette Encoding E. coli Ferritin Under the Control of the Vaccinia P_{SEL} Promoter into the Vaccinia HA Locus

Vector pNCVVhaT-ftn (SEQ ID NO: 47) was employed to develop strains GLV-1h82 and GLV-1h83 having the following genotypes:, F14.5L: (P_{SEL})Ruc-GFP, TK: (P_{SEL})TrfR-(P_{7.5k})LacZ (strain GLV-1h82), HA: (P_{SEL})ftn, and F14.5L: (P_{SEL})Ruc-GFP, TK: (P_{SEL})rTrfR-(P_{7.5k})LacZ (strain GLV-1h83), HA: (P_{SEL})ftn. Strains GLV-1h82 and GLV-1h83 were generated by inserting DNA encoding E. coli ferritin (ftn) (SEQ ID NO: 48) operably linked to the vaccinia virus synthetic early/late promoter (P_{SEL}) (SEQ ID NO: 29) into the HA locus of starting strains GLV-1h22 and GLV-1h68, respectively, thereby deleting the gusA expression cassette at the HA locus of these starting strains. Vector pNCVVhaT-ftn contains a DNA fragment encoding E. coli ferritin operably linked to the vaccinia synthetic early/late promoter(P_{SEL}), sequences of the HA gene flanking the (P_{SEL})ftn DNA fragment, the E. coli guanine phosphoribosyltransferase (gpt) gene under the control of the vaccinia virus P_{7.5kE} promoter for transient dominant selection of virus that has incorporated the vector, and sequences of the pUC plasmid.

To generate vector pNCVVhaT-ftn, DNA encoding E. coli ferritin (ftn) was amplified from genomic DNA of E. coli Top10 (Invitrogen, Carlsbad, Calif.) using the following primers:

5′SSEL-ftn-VV3
(SEQ ID NO: 49)
(5′-AAAGATAAGCTTAAAAATTGAAATTTTATTTTTTTTTTTTGGAATA
TAAATACCATGCTGAAACCAGAAATGATTGAA-3′)
and
(SEQ ID NO: 50)
3′ ftn-VV2
(5′-ATAATAGGATCCTTAGTTTTGTGTGTCGAGGGT-3′).

Primer 5′SSEL-ftn-VV3 introduces a HindIII site, the P_{SEL} promoter sequence for vaccinia virus synthetic strong early/late expression, and a Kozak sequence (ACC) in front of the start codon of ftn. 3′ ftn-VV2 introduces a BamHI restriction site. The PCR product as well as the plasmid pNCVVhaT (SEQ ID NO: 4) were digested with BamHI and HindIII, ligated, and transformed into E. coli Top10 to yield pNCVVhaT-ftn ID NO: 47). This final cloning step places the (P_{SEL})ftn expression cassette between the left and right HA gene flanking sequences in pNCVVhaT and eliminates the non-coding DNA that is located between these flanking sequences in pNCVVhaT.

f. pCR-TK-SEL-mRFP1: for Insertion of an Expression Cassette Encoding a Fusion Protein of CBG99 and mRFP1 Under the Control of the Vaccinia P_{SEL} Promoter into the Vaccinia TK Locus

Vector pCR-TK-SEL-mRFP1 (SEQ ID NO: 51) was employed to develop strain GLV-1h84 having the following genotype: F14.5L: BamHI-HindIII, TK: (P_{SEL})CBG99-mRFP1, HA: HindIII-BamHI. Strain GLV-1h84 was generated by inserting DNA encoding a fusion protein of CBG99 (green-emitting click beetle luciferase) and mRFP1 (red fluorescent protein) linked through a picornavirus 2A element (SEQ ID NO: 52) operably linked to the vaccinia virus synthetic early/late promoter (P_{SEL}) (SEQ ID NO: 29) into the TK locus of strain GLV-1h73 thereby deleting the rTrfR-LacZ expression cassette at the TK locus of strain GLV-1h73. Vector pCR-TK-SEL-mRFP1 contains a DNA fragment encoding a CBG99-mRFP1 fusion protein operably linked to the vaccinia synthetic early/late promoter (P_{SEL}), sequences of the TK gene flanking the (P_{SEL})-fusion protein-encoding DNA fragment, the E. coli guanine phosphoribosyltransferase (gpt) gene under the control of the vaccinia virus P7.5k early and late promoter for transient dominant selection of virus that has incorporated the vector, and sequences of the pUC plasmid.

To generate vector pCR-TK-SEL-mRFP1, cDNA encoding the fusion protein CBG99 (green-emitting click beetle luciferase) and mRFP1 (red fluorescent protein) linked through the picornavirus 2A element was PCR amplified from CBG99-2A-mRFP1 (SEQ ID NO: 53) with the primers:

(SEQ ID NO: 54)
mRFP5 (5′-GTCGACGCCACCATGGTGAAGCGTGAG-3′)
and
(SEQ ID NO: 55)
mRFP3 (5′-TCATTAGGCGCCGGTGGAGT-3′).

The PCR product was cloned into vector pCR-Blunt II-TOPO (Invitrogen; SEQ ID NO: 40) to yield pCRII-mRFP (SEQ ID NO: 56). After confirming the sequence, the CBG99-mRFP1 fusion cDNA molecule (SEQ ID NO: 52) was released by SalI and EcoRV restriction enzyme digest and inserted into pCR-SEL4 (SEQ ID NO: 33), precut with SalI and SmaI to generate plasmid pCR-SEL-mRFP1 (SEQ ID NO: 57). (pCR-SEL4 was constructed as follows: The cDNA spanning the synthetic early/late promoter P_{SEL} (SEQ ID NO: 29) for vaccinia virus and the multiple cloning site (MCS) region in pSC65 (SEQ ID NO: 30) was PCR amplified with the primers SEL5 (5′-TAGAGCTCGGTTTGGAATTAGTGAAAGC-3′) (SEQ ID NO: 31) and SEL3 (5′-TAGAGCTCTCCAGACATTGTTGAATTAG-3′) (SEQ ID NO: 32), and cloned into the TA cloning site of vector pCR2.1 to yield pCR-SEL4 (SEQ ID NO: 33)). This intermediate cloning step placed the fusion cDNA molecule under the control of vaccinia virus synthetic early/late promoter (P_{SEL}). The SEL-CBG99-mRFP1 expression cassette was then released by Sad digestion and cloned into the same-cut vaccinia virus TK locus transfer vector pCR-TKLR-gpt2 (SEQ ID NO: 17) to give the final construct pCR-TK-SEL-mRFP1 (SEQ ID NO: 51). This final cloning step placed the (P_{SEL})CBG99-mRFP1 expression cassette between the left and right TK gene flanking sequences in pCR-TKLR-gpt2 and eliminated the non-coding DNA that is located between these flanking sequences in pCR-TKLR-gpt2.

g. pCR-gpt-dA35R6: for Deletion of the A35R Locus and Insertion of a Non-Coding Heterologous DNA with Multiple Cloning Sites

Vector pCR-gpt-dA35R-6 (SEQ ID NO: 89) was employed to create vaccinia strains GLV-1j87, GLV-1j88 and GLV-1j89, having the following genotypes: F14.5L: (P_{SEL})Ruc-GFP, TK: (P_{SEL})rTrfR-(P_{7.5k})LacZ, HA: (P₁₁ₖ)gusA, A35R: deleted, multiple cloning sites (MCS) (strain GLV-1j87); F14.5L: BamHI-HindIII, TK: (P_{SEL})rTrfR-(P_{7.5k})LacZ, HA: HindIII-BamHI, A35R: deleted, MCS (strain GLV-1j88); and F14.5L: BamHI-HindIII, TK: SacI-BamHI, HA: HindIII-BamHI, A35R: deleted, MCS (strain GLV-1j89). Strains GLV-1j87, GLV-1j88 and GLV-1j89, were generated by inserting a short DNA fragment with multiple cloning sites (HindIII, Sad and BamHI) into the A35R locus of strains GLV-1h68, GLV-1h73 and GLV-1h74, respectively, thereby creating a fusion of the flanking A34R and A36R regions and deleting the A35R gene. Vector pCR-gpt-dA35R-6 contains a non-coding DNA fragment with multiple cloning sites flanked by sequences that flank the A35R gene (a fusion of A34R and A36R regions) and the E. coli guanine phosphoribosyltransferase (gpt) gene under the control of the vaccinia virus P_{7.5kE} promoter for transient dominant selection of virus that has incorporated the vector.

The left and right flanking sequences of A35R, the A34R and A36R regions, were PCR amplified. The A34R gene region was PCR amplified with primers

A34R-L: 5′-ATCTCGAGTGAGGATACATGGGGATCTGATG-3′ (SEQ ID NO: 66) and

A34R-R: 5′-ATGAGCTCCCGGGAAGCTTGGCGGCGTACGTTAACGAC-3′ (SEQ ID NO: 67), using LIVP genomic DNA (SEQ ID NO: 2) as the template.

The A36R gene region was PCR amplified with primers

A36R-L: 5′-ATGAGCTCGGATCCTGCATATCAGACGGCAATGG-3′ (SEQ ID NO: 68) and

A36R-R: 5′-ATGGGCCCATCGCTATGTGCTCGTCTA-3′ (SEQ ID NO: 69), using LIVP genomic DNA (SEQ ID NO: 2) as the template.

The A34R and A36R PCR products were digested with Sad, and the restricted products were then purified and ligated together. The A34R and A36R ligation product was used as the template for PCR amplification of the A34R and A36R fusion cDNA, with primers A34R-L and A36R-R. The amplified fusion cDNA was cloned into pCR-Blunt II-TOPO vector (Invitrogen; SEQ ID NO: 40) to generate vector pCRII-dA35R-1 (SEQ ID NO: 87). The resulting vector was confirmed by sequencing.

A p7.5-gpt expression vector with the HindIII, SacI and BamHI sites removed was then generated. The TK region in the TK locus transfer vector pCR-TKLR-gpt2 (SEQ ID NO: 17) was removed with HindIII and SpeI digestion. The vector fragment was blunt ended with Klenow treatment, and then ligated to generate construct pCR-dTK-gpt1 (SEQ ID NO: 88). The restriction sites HindIII, SacI and BamHI are removed in the resulting pCR-dTK-gpt1 vector (SEQ ID NO: 88).

To generate pCR-gpt-dA35R-6, the A34R and A36R fusion cDNA was released from pCRII-dA35R-1 (SEQ ID NO: 87) by XhoI and ApaI digestion, and inserted into vector pCR-dTK-gpt1 (SEQ ID NO: 88), precut with XhoI and ApaI. The resulting construct pCR-gpt-dA35R-6 (SEQ ID NO: 89) was confirmed by sequencing.

h. HA-SE-IL-6-1: for Insertion of an Expression Cassette Encoding sIL-6R/IL-6 Under the Control of the Vaccinia P_{SE} Promoter into the Vaccinia HA Locus.

Vector HA-SE-IL-6-1 (SEQ ID NO: 77) was employed to develop strain GLV-1h90 having the following genotype: F14.5L: (P_{SEL})Ruc-GFP, TK: (P_{SEL})rTrfR-(P_{7.5k})LacZ, HA: (P_{SE})sIL-6R/IL-6. Strain GLV-1h90 was generated by inserting DNA encoding a fusion protein of human IL-6 (encoding amino acids 29˜212) fused to the human soluble IL-6 receptor (sIL-6R) (amino acids 1˜323) by a linker sequence (encoding RGGGGSGGGGSVE (SEQ ID NO: 90); complete sequence of sIL-6R/IL insert (SEQ ID NO: 106)) operably linked to the vaccinia virus synthetic early promoter (P_{SE}) (SEQ ID NO: 35) into the HA locus of strain GLV-1h68, thereby deleting the gusA expression cassette at the HA locus of starting GLV-1h68. Vector HA-SE-IL-6-1 contains a DNA fragment encoding the sIL-6R/IL-6 fusion protein operably linked to the vaccinia synthetic early promoter (P_{SE}) and sequences of the HA gene flanking the (P_{SE})-fusion protein-encoding DNA fragment.

Plasmid pCR-SE1 (SEQ ID NO: 36), containing the vaccinia synthetic early promoter, i.e., P_{SE}, was used as the source of the vaccinia synthetic early promoter in generating vector HA-SE-IL-6-1. pCR-SE1 was constructed as follows. The multiple cloning site (MCS) region in pSC65 (Moss and Earl, Current Protocols in Molecular Biology, 16.17.4, 1998; SEQ ID NO: 30) was PCR amplified with the primers:

SE5:

5′-TAGAGCTCAAAAATTGAAAAACTAGCGTCTTTTTTTGCTCGAAGTCGAC AGATCTAGGCCTG-3′ (SEQ ID NO: 34), containing the sequence for synthetic early promoter P_{SE} (SEQ ID NO: 35), and

SEL3:

5′-TAGAGCTCTCCAGACATTGTTGAATTAG-3′(SEQ ID NO: 32).

The resulting PCR product was inserted into the TA cloning site of vector pCR2.1 to obtain pCR-SE1 (SEQ ID NO: 36).

To generate vector HA-SE-IL-6-1, cDNA encoding the fusion protein sIL-6R/IL-6 was PCR amplified from pCDM8-H-IL-6 (U.S. Pat. No. 7,112,436) with the primers:

(SEQ ID NO: 62)
5′-GTCGACCCACCATGCTGGCCGTCGGCTGCGC-3′
and
(SEQ ID NO: 63)
5′-GGTACCCTAGAGTCGCGGCCGCGACC-3′.

The PCR product was cloned into vector pCR-Blunt II-TOPO (Invitrogen; SEQ ID NO: 40) to yield pCRII-IL6-3 (SEQ ID NO: 73). After confirming the sequence, the sIL-6R/IL-6 fusion cDNA molecule (SEQ ID NO: 106) was released by KpnI (blunt ended) and SalI restriction enzyme digest and inserted into vector pCR-SE1 (SEQ ID NO: 36), precut with SalI and SmaI to generate plasmid pCR-SE-IL6-7 (SEQ ID NO: 74), thus placing the IL-6 fusion cDNA under the control of vaccinia virus synthetic early (SE) promoter.

The cDNA of SE-IL6 was released from pCR-SE-IL6-7 (SEQ ID NO: 74) by HindIII and BamHI restriction enzyme digest and inserted into the HA transfer vector, pNCVVhaT (SEQ ID NO: 4), precut with HindIII and BamHI to generate plasmid HA-SE-IL6-1 (SEQ ID NO: 77). The SL-sIL-6R/IL-6 fusion expression was confirmed by sequencing.

i. HA-SEL-IL-6-1: for Insertion of an Expression Cassette Encoding sIL-6R/IL-6 Under the Control of the Vaccinia P_{SEL} Promoter into the Vaccinia HA Locus.

Vector HA-SEL-IL-6-1 (SEQ ID NO: 79) was employed to develop strain GLV-1h91 having the following genotype: F14.5L: (P_{SEL})Ruc-GFP, TK: (P_{SEL})rTrfR-(P_{7.5k})LacZ, HA: (P_{SEL})sIL-6R/IL-6. Strain GLV-1h91 was generated by inserting DNA encoding the sIL-6R/IL-6 fusion protein operably linked to the vaccinia virus synthetic early/late promoter (P_{SEL}) (SEQ ID NO: 29) into the HA locus of starting strain GLV-1h68, thereby deleting the gusA expression cassette at the HA locus of starting GLV-1h68. Vector HA-SL-IL-6-1 contains a DNA fragment encoding the sIL-6R/IL-6 fusion protein operably linked to the vaccinia synthetic early promoter (P_{SEL}) and sequences of the HA gene flanking the (P_{SEL})-fusion protein-encoding DNA fragment.

Plasmid pCR-SEL4 (SEQ ID NO: 33; see (f) above for construction of pCR-SEL4), containing the vaccinia synthetic early/late promoter, i.e., P_{SEL}, was used as the source of the vaccinia synthetic early/late in generating vector HA-SEL-IL-6-1.

To generate vector HA-SL-IL-6-1, the sIL-6R/IL-6 fusion cDNA molecule (SEQ ID NO: 106) was released from vector pCRII-IL6-3 (see (h) above; SEQ ID NO: 73) by KpnI and SalI restriction enzyme digest and inserted into vector pCR-SEL4 (SEQ ID NO: 33), precut with SalI and SmaI to generate plasmid pCR-SEL-IL6-2 (SEQ ID NO: 76), thus placing the IL-6 fusion cDNA under the control of vaccinia virus synthetic early/late (SEL) promoter.

The cDNA of SEL-IL6 was released from pCR-SEL-IL6-2 (SEQ ID NO: 76) by HindIII restriction enzyme digest and inserted into the HA transfer vector, pNCVVhaT (SEQ ID NO: 4), precut with HindIII to generate plasmid HA-SEL-IL6-1 (SEQ ID NO: 79). The SEL-sIL-6R/IL-6 fusion expression cassette was confirmed by sequencing.

j. HA-SL-IL-6-1: for Insertion of an Expression Cassette Encoding sIL-6R/IL-6 Under the Control of the Vaccinia P_{SL} Promoter into the Vaccinia HA Locus.

Vector HA-SL-IL-6-1 (SEQ ID NO: 78) was employed to develop strain GLV-1h92 having the following genotype: F14.5L: (P_{SEL})Ruc-GFP, TK: (P_{SEL})rTrfR-(P_{7.5k})LacZ, HA: (P_{SL})sIL-6R/IL-6. Strain GLV-1h92 was generated by inserting DNA encoding the sIL-6R/IL-6 fusion protein (SEQ ID NO: 106) operably linked to the vaccinia virus synthetic late promoter (P_{SL}) (SEQ ID NO: 38) into the HA locus of starting strain GLV-1h68, thereby deleting the gusA expression cassette at the HA locus of starting GLV-1h68. Vector HA-SL-IL-6-1 contains a DNA fragment encoding the sIL-6R/IL-6 fusion protein operably linked to the vaccinia synthetic late promoter (P_{SL}) and sequences of the HA gene flanking the (P_{SL})-fusion protein-encoding DNA fragment.

Plasmid pCR-SL3 (SEQ ID NO: 39), containing the vaccinia synthetic late promoter, i.e., P_{SL}, was used as the source of the vaccinia synthetic late promoter in generating vector HA-SL-IL-6-1 (SEQ ID NO: 78). To construct pCR-SL3, the MCS region in pSC65 was PCR amplified with the primers:

SL5:

5′-TAGAGCTCTTTTTTTTTTTTTTTTTTTT GGCATATAAATAAGTCGA CAGATCTAGGCCTG-3′ (SEQ ID NO: 37), containing the sequence for synthetic late promoter P_{SL} (SEQ ID NO: 38), and

SEL3:

5′-TAGAGCTCTCCAGACATTGTTGAATTAG-3′) (SEQ ID NO: 32). The resulting PCR product was cloned into the TA cloning site of vector pCR2.1 to yield pCR-SL3 (SEQ ID NO: 39).

To generate vector HA-SL-IL-6-1, the sIL-6R/IL-6 fusion cDNA molecule (SEQ ID NO: 106) was released from vector pCRII-IL6-3 (see (h) above; SEQ ID NO: 73) by KpnI and SalI restriction enzyme digest and inserted into vector pCR-SL3 (SEQ ID NO: 39), precut with SalI and SmaI to generate plasmid pCR-SL-IL6-2 (SEQ ID NO: 75), thus placing the IL-6 fusion cDNA under the control of vaccinia virus synthetic late (SL) promoter.

The cDNA of SL-sIL-6R/IL-6 was released from pCR-SL-IL6-2 (SEQ ID NO: 75) by HindIII and BamHI restriction enzyme digest and inserted into the HA transfer vector, pNCVVhaT (SEQ ID NO: 4), precut with HindIII and BamHI to generate plasmid HA-SL-IL6-1 (SEQ ID NO: 78). The SL-sIL-6R/IL-6 fusion expression cassette was confirmed by sequencing.

k. FSE-IL-24: for Insertion of an Expression Cassette Encoding IL-24 Under the Control of the Vaccinia P_{SE} Promoter into the Vaccinia F14.5L Locus.

Vector FSE-IL-24 (SEQ ID NO: 84) was employed to develop strain GLV-1h96 having the following genotype: F14.5L: (P_{SE})IL-24, TK: (P_{SEL})rTrfR-(P_{7.5k})LacZ, HA: (P₁₁ₖ)gusA. Strain GLV-1h96 was generated by inserting DNA encoding human IL-24 operably linked to the vaccinia virus synthetic early promoter (P_{SE}) (SEQ ID NO: 35) into the F14.5L locus of strain GLV-1h68, thereby deleting the Ruc-GFP fusion gene expression cassette at the F14.5L locus of GLV-1h68. Vector FSE-IL-24 contains a DNA fragment encoding the IL-24 protein operably linked to the vaccinia synthetic early promoter (P_{SE}) and sequences of the F14.5L gene flanking the (P_{SE})-fusion protein-encoding DNA fragment.

Plasmid pCR-SE1 (SEQ ID NO: 36; see (h) above for description of pCR-SE1), containing the vaccinia synthetic early promoter, i.e., P_{SE}, was used as the source of the vaccinia synthetic early promoter in generating vector FSE-IL-24.

To generate vector FSE-IL-24, cDNA encoding the human IL-24 was PCR amplified from cDNA clone MGC:8926 (complete cds from Origene Trueclone collection) with the primers:

(SEQ ID NO: 64)
5′-GTCGACCACCATGAATTTTCAACAGAGGCTGC-3′
and
(SEQ ID NO: 65)
5′-CCCGGGTTATCAGAGCTTGTAGAATTTCTGCATC-3′.

The PCR product was cloned into vector pCR-Blunt II-TOPO (Invitrogen; SEQ ID NO: 40) to yield pCR11-IL24-3 (SEQ ID NO: 80). After confirming the sequence, the IL-24 cDNA molecule (SEQ ID NO: 107) was released by SalI and SmaI digestion and inserted into vector pCR-SE1 (SEQ ID NO: 36), precut with SalI and SmaI to generate plasmid pCR-SE-IL24-2 (SEQ ID NO: 81), thus placing the IL-24 cDNA under the control of vaccinia virus synthetic early (SE) promoter.

The cDNA of SE-IL24 was released from pCR-SE-IL24-2 (SEQ ID NO: 81) by HindIII and BamHI restriction enzyme digest and inserted into the F14.5L transfer vector, pNCVVf14.51T (SEQ ID NO: 11), precut with HindIII and BamHI to generate plasmid FSE-IL24-1 (SEQ ID NO: 84). The SL-IL-24 expression was confirmed by sequencing.

l. FSEL-IL-24: for Insertion of an Expression Cassette Encoding IL-24 Under the Control of the Vaccinia P_{SEL} Promoter into the Vaccinia F14.5L Locus.

Vector FSEL-IL24-1 (SEQ ID NO: 86) was employed to develop strain GLV-1h97 having the following genotype: F14.5L: (P_{SEL})IL-24, TK: (P_{SEL})rTrfR-(P_{7.5k})LacZ, HA: (P₁₁ₖ)gusA. Strain GLV-1h97 was generated by inserting DNA human IL-24 operably linked to the vaccinia virus synthetic early/late promoter (P_{SEL}) (SEQ ID NO: 29) into the F14.5L locus of strain GLV-1h68, thereby deleting the Ruc-GFP fusion gene expression cassette at the F14.5L locus of GLV-1h68. Vector FSEL-IL24-1 contains a DNA fragment encoding the IL-24 protein operably linked to the vaccinia synthetic early/late promoter (P_{SEL}), sequences of the F14.5L gene flanking the (P_{SEL})-fusion protein-encoding DNA fragment, the E. coli guanine phosphoribosyltransferase (gpt) gene under the control of the vaccinia virus P7.5k early and late promoter for transient dominant selection of virus that has incorporated the vector, and sequences of the pUC plasmid.

Plasmid pCR-SEL4 (SEQ ID NO: 33; see (f) above for construction of pCR-SEL4), containing the vaccinia synthetic early/late promoter, i.e., P_{SEL}, was used as the source of the vaccinia synthetic early/late in generating vector FSEL-IL24-1.

To generate vector FSEL-IL24-1, the IL-24 cDNA molecule (SEQ ID NO: 107) was released from vector pCRII-IL24-3 (see (n) above; SEQ ID NO: 80) by KpnI and SalI restriction enzyme digest and inserted into vector pCR-SEL4 (SEQ ID NO: 33), precut with SalI and SmaI to generate plasmid pCR-SEL-IL24-2 (SEQ ID NO: 83), thus placing the IL-6 fusion cDNA under the control of vaccinia virus synthetic early/late (SEL) promoter.

The cDNA of SEL-IL24 was released from pCR-SEL-IL24-2 (SEQ ID NO: 83) by HindIII restriction enzyme digest and inserted into the F14.5L transfer vector, pNCVVf14.51T (SEQ ID NO: 11), precut with HindIII to generate plasmid FSEL-IL24-1 (SEQ ID NO: 86). The SEL-IL-24 expression cassette was confirmed by sequencing.

m. FSL-IL-24: for Insertion of an Expression Cassette Encoding IL-24 Under the Control of the Vaccinia P_{SL} Promoter into the Vaccinia F14.5L Locus.

Vector FSL-IL24-1 (SEQ ID NO: 85) was employed to develop strain GLV-1h98 having the following genotype: F14.5L: (P_{SL})IL-24, TK: (P_{SEL})rTrJR-(P_{7.5k})LacZ, HA: (P₁₁ₖ)gusA. Strain GLV-1h98 was generated by inserting DNA encoding the human IL-24 protein operably linked to the vaccinia virus synthetic late promoter (P_{SL}) (SEQ ID NO: 38) into the F14.5L locus of starting strain GLV-1h68, thereby deleting the Ruc-GFP fusion gene expression cassette at the F14.5L locus of GLV-1h68. Vector FSL-IL24-1 contains a DNA fragment encoding the IL-24 protein operably linked to the vaccinia synthetic late promoter (P_{SL}) and sequences of the F14.5L gene flanking the (P_{SL})-fusion protein-encoding DNA fragment.

Plasmid pCR-SL3 (SEQ ID NO: 39; see (j) above for description of pCR-SL3), containing the vaccinia synthetic late promoter, i.e., P_{SL}, was used as the source of the vaccinia synthetic late promoter in generating vector FSL-IL24-1.

To generate vector FSL-IL24-1, the IL-24 cDNA molecule (SEQ ID NO: 107) was released from vector pCRII-IL24-3 (see (n) above; SEQ ID NO: 80) by KpnI and SalI restriction enzyme digest and inserted into vector pCR-SL3 (SEQ ID NO: 39), precut with SalI and SmaI to generate plasmid pCR-SL-IL24-2 (SEQ ID NO: 82), thus placing the IL-24 fusion cDNA under the control of vaccinia virus synthetic late (SL) promoter.

The cDNA of SL-IL-24 was released from pCR-SL-IL24-2 (SEQ ID NO: 82) by HindIII and BamHI restriction enzyme digest and inserted into the F14.5L transfer vector, pNCVVf14.51T (SEQ ID NO: 11), precut with HindIII and BamHI to generate plasmid FSL-IL24-1 (SEQ ID NO: 85). The SL-IL-24 expression cassette was confirmed by sequencing.

n. pCR-TK-SE-tTF-RGD: for Insertion of an Expression Cassette Encoding the tTF-RGD Fusion Protein Under the Control of the Vaccinia P_{SE} Promoter into the Vaccinia TK Locus

Vector pCR-TK-SE-tTF-RGD (SEQ ID NO: 95) was employed to develop strain GLV-1h104 having the following genotype: F14.5L: (P_{SEL})Ruc-GFP; TK: (P_{SE})tTF-RGD; HA: (P₁₁ₖ)gusA. Strain GLV-1h104 was generated by inserting DNA encoding a tTF-RGD fusion protein (SEQ ID NO: 92 (DNA sequence); SEQ ID NO: 93 (amino acid sequence)) into the TK locus of strain GLV-1h68 thereby deleting the rTrfR-LacZ expression cassette at the TK locus of strain GLV-1h68. Vector pCR-TK-SE-tTF-RGD contains a DNA fragment encoding the tTF-RGD fusion protein operably linked to the vaccinia synthetic early promoter (P_{SE}), sequences of the TK gene flanking the (P_{SE})-fusion protein-encoding DNA fragment, the E. coli guanine phosphoribosyltransferase (gpt) gene under the control of the vaccinia virus P7.5k early and late promoter for transient dominant selection of virus that has incorporated the vector, and sequences of the pUC plasmid.

cDNA encoding human tissue factor (huTF) was synthesized from RNA extracted from MCF-7 cells (Qiagen RNA extraction kit). The huTF cDNA was synthesized from the RNA in a reverse transcriptase reaction (Invitrogen Superscript II cDNA synthesis kit) using primer hu-tTF-RGD-rev-cDNA, which binds to a region upstream of the huTF sequence:

hu-tTF-RGD-rev-cDNA

| | |
|---|---|
| 5′-CTTTCTACACTTGTGTAGAGATATAGC-3′ | (SEQ ID NO: 91) |

After cDNA synthesis, the tTF-RGD fragment was PCR amplified (Invitrogen Accu Prime pa Supermix) using hu-TF cDNA as a template and the following primers:

hu-tTF-RGD-for (SalI)

5′-GTCGACCCACCATGGAGACCCCTGCCTG-3′ (SEQ ID NO: 115) and

hu-tTF-RGD-rev (PacI)

5′-TTAATTAATATTATGGAGAATCACCTCTTCCTCTGAATTCCCCTT TCTCCTGG-3′ (SEQ ID NO: 116). The hu-tTF-RGD-rev primer contains additional restriction endonuclease sites and the sequence of the RGD binding motif.

The PCR product was cloned into vector pCR-Blunt II-TOPO (Invitrogen; SEQ ID NO: 40) via blunt end ligation (Quick Ligation Kit; New England Biolabs) to yield pCRII-tTF-RGD (SEQ ID NO: 94). The tTF-RGD cDNA molecule (SEQ ID NO: 92) was confirmed by sequencing.

The vaccinia synthetic early promoter, i.e., P_{SE}, and flanking TK gene regions of pCR-TK-SE-tTF-RGD are derived from an intermediate plasmid, TK-SE-CSF-2 (SEQ ID NO: 110), which contains the cDNA for GM-CSF under the control of the vaccinia synthetic early promoter flanked by the TK gene regions. pCR-SE1 (SEQ ID NO: 36; see (h) above for description of pCR-SE1), containing the vaccinia synthetic early promoter, i.e., P_{SE}, was used as the source of the vaccinia synthetic early promoter in generating vector TK-SE-CSF-2. The cDNA encoding GM-CSF protein (mouse granulocyte-macrophage colony-stimulating factor) was PCR amplified from pPICZA-mGM-CSF (SEQ ID NO: 72) with the primers GM-CSF5 5′-CTAGTCGACATGTGGCTGCAGAATTTACTTTTCCTGGGCATTGTGGTCT ACAGCCTCTCAGCACCCACCCGCTCACCCATC-3′ (SEQ ID NO: 70), containing the signal peptide sequence, and

GM-CSF3

5′-GGGTCATTTTTGGACTGGTTTTT-3′ (SEQ ID NO: 71), containing a stop codon. The PCR amplification product was cloned into vector pCR-Blunt II-TOPO (SEQ ID NO: 40; Invitrogen, Carlsbad, Calif.). The resulting vector pCRII-CSF9 (SEQ ID NO: 108), which contained the correct insert, was digested with SalI and EcoRI (blunt-ended after digestion), and the released GM-CSF cDNA was cloned into vector pCR-SE1 (SEQ ID NO: 36) precut with SalI and SmaI, resulting in SE-CSF-2 (SEQ ID NO: 109). Thus, SE-CSF-2 contains the vaccinia synthetic early promoter (P_{SE}) operably linked to DNA encoding GM-CSF. The GM-CSF expression cassette containing GM-CSF cDNA under the control of the P_{SE} was excised from SE-CSF-2 by SacI digestion and cloned into the same-cut vector pCR-TKLR-gpt2 (SEQ ID NO: 17) to generate the construct TK-SE-CSF-2 (SEQ ID NO: 110). This cloning step places the (P_{SE})GM-CSF expression cassette between the left and right TK gene flanking sequences in pCR-TKLR-gpt2 and eliminates the non-coding DNA that is located between these flanking sequences in pCR-TKLR-gpt2.

To generate vector pCR-TK-SE-tTF-RGD, the tTF-RGD fragment was released by SalI and PacI restriction enzyme digest of pCRII-tTF-RGD (SEQ ID NO: 94) and inserted into TK-SE-CSF-2 (SEQ ID NO: 110), precut with SalI and Pad, to generate plasmid pCR-TK-SE-tTF-RGD (SEQ ID NO: 95), thus placing the tTF-RGD cDNA under the control of vaccinia virus synthetic early (P_{SE}) promoter and in between the left and right TK gene flanking sequences. The tTF-RGD cDNA insert was confirmed by sequencing.

o. pCR-TK-SEL-tTF-RGD: for Insertion of an Expression Cassette Encoding the tTF-RGD Fusion Protein Under the Control of the Vaccinia P_{SEL} Promoter into the Vaccinia TK Locus

Vector pCR-TK-SEL-tTF-RGD (SEQ ID NO: 96) was employed to develop strain GLV-1h105 having the following genotype: F14.5L: (P_{SEL})Ruc-GFP; TK: (P_{SEL})tTF-RGD; HA: (P₁₁ₖ)gusA. Strain GLV-1h105 was generated by inserting DNA encoding a tTF-RGD fusion protein (SEQ ID NO: 92) into the TK locus of strain GLV-1h68 thereby deleting the rTrfR-LacZ expression cassette at the TK locus of strain GLV-1h68. Vector pCR-TK-SEL-tTF-RGD contains a DNA fragment encoding the tTF-RGD fusion protein operably linked to the vaccinia synthetic early/late promoter (P_{SEL}), sequences of the TK gene flanking the (P_{SEL})-fusion protein-encoding DNA fragment, the E. coli guanine phosphoribosyltransferase (gpt) gene under the control of the vaccinia virus P7.5k early and late promoter for transient dominant selection of virus that has incorporated the vector and sequences of the pUC plasmid.

The vaccinia synthetic early/late promoter, i.e., P_{SEL}, and flanking TK gene regions of pCR-TK-SEL-tTF-RGD are derived from an intermediate plasmid, TK-SEL-CSF-2 (SEQ ID NO: 112), which contains the cDNA for GM-CSF under the control of the vaccinia synthetic early/late promoter flanked by the TK gene regions. Plasmid pCR-SEL4 (SEQ ID NO: 33; see (f) above for construction of pCR-SEL4), containing the vaccinia synthetic early/late promoter, i.e., P_{SEL}, was used as the source of the vaccinia synthetic early/late in generating vector TK-SEL-CSF-2. DNA encoding GM-CSF was excised from pCRII-CSF9 (SEQ ID NO: 108) with SalI and EcoRI (blunt-ended after digestion), and cloned into vector pCR-SEL4 (SEQ ID NO: 33) precut with SalI and SmaI, resulting in SEL-CSF-2 (SEQ ID NO: 111). Thus, SEL-CSF-2 contains the vaccinia synthetic early/late promoter (P_{SEL}) operably linked to DNA encoding GM-CSF. The GM-CSF expression cassette containing DNA encoding GM-CSF under the control of P_{SEL} was then excised from SEL-CSF-2 by SacI digestion and cloned into the same-cut vector pCR-TKLR-gpt2 (SEQ ID NO: 17) to generate the construct TK-SEL-CSF-2 (SEQ ID NO: 112). This cloning step places the (P_{SEL})GM-CSF expression cassette between the left and right TK gene flanking sequences in pCR-TKLR-gpt2 and eliminates the non-coding DNA that is located between these flanking sequences in pCR-TKLR-gpt2.

To generate vector pCR-TK-SEL-tTF-RGD, the tTF-RGD fragment was released by SalI and PacI restriction enzyme digest of pCRII-tTF-RGD (see (n) above; SEQ ID NO: 94) and inserted into TK-SEL-CSF-2 (SEQ ID NO: 112), precut with SalI and PacI to generate plasmid pCR-TK-SEL-tTF-RGD (SEQ ID NO: 96), thus placing the tTF-RGD cDNA under the control of vaccinia virus synthetic early/late (P_{SEL}) promoter and in between the left and right TK gene flanking sequences. The tTF-RGD cDNA insert was confirmed by sequencing.

p. pCR-TK-SL-tTF-RGD: for Insertion of an Expression Cassette Encoding the tTF-RGD Fusion Protein Under the Control of the Vaccinia P_{SL} Promoter into the Vaccinia TK Locus

Vector pCR-TK-SL-tTF-RGD (SEQ ID NO: 97) was employed to develop strain GLV-1h106 having the following genotype: F14.5L: (P_{SEL})Ruc-GFP; TK: (P_{SL})tTF-RGD; HA: (P₁₁ₖ)gusA. Strain GLV-1h106 was generated by inserting DNA encoding a tTF-RGD fusion protein (SEQ ID NO: 92) into the TK locus of strain GLV-1h68 thereby deleting the rTrfR-LacZ expression cassette at the TK locus of strain GLV-1h68. Vector pCR-TK-SL-tTF-RGD contains a DNA fragment encoding the tTF-RGD fusion protein operably linked to the vaccinia synthetic late promoter (_{SL}), sequences of the TK gene flanking the (P_{SL})-fusion protein-encoding DNA fragment, the E. coli guanine phosphoribosyltransferase (gpt) gene under the control of the vaccinia virus P7.5k early and late promoter for transient dominant selection of virus that has incorporated the vector, and sequences of the pUC plasmid.

The vaccinia synthetic late promoter, i.e., P_{SL}, and flanking TK gene regions of pCR-TK-SL-tTF-RGD are derived from an intermediate plasmid, TK-SL-CSF-2 (SEQ ID NO: 114), which contains the cDNA for GM-CSF under the control of the vaccinia synthetic late promoter flanked by the TK gene regions.

Plasmid pCR-SL3 (SEQ ID NO: 39; see (j) above for description of pCR-SL3), containing the vaccinia synthetic late promoter, i.e., P_{SL}, was used as the source of the vaccinia synthetic late promoter in generating vector TK-SL-CSF-3 (SEQ ID NO: 114). DNA encoding mouse GM-CSF was excised from pCRII-CSF9 (SEQ ID NO: 108) with SalI and EcoRI (blunt-ended after digestion), and cloned into vector pCR-SL3 (SEQ ID NO: 39) precut with SalI and SmaI, resulting in SL-CSF-2 (SEQ ID NO: 113). Thus, SL-CSF-2 contains the vaccinia synthetic late promoter (P_{SL}) operably linked to DNA encoding GM-CSF. The GM-CSF expression cassette containing DNA encoding GM-CSF under the control of the PSL was excised out from SL-CSF-2 by Sac I and cloned into the same-cut vector pCR-TKLR-gpt2 (SEQ ID NO: 17) to generate the construct TK-SL-CSF-3 (SEQ ID NO: 114). This cloning step places the (P_{SL})GM-CSF expression cassette between the left and right TK gene flanking sequences in pCR-TKLR-gpt2 and eliminates the non-coding DNA that is located between these flanking sequences in pCR-TKLR-gpt2.

To generate vector pCR-TK-SL-tTF-RGD, the tTF-RGD fragment was released by SalI and PacI restriction enzyme digest of pCR11-tTF-RGD (see (n) above; SEQ ID NO: 94) and inserted into TK-SL-CSF-3 (SEQ ID NO: 114), precut with SalI and PacI to generate plasmid pCR-TK-SL-tTF-RGD (SEQ ID NO: 97), thus placing the tTF-RGD cDNA under the control of vaccinia virus synthetic late (P_{SL}) promoter and in between the left and right TK gene flanking sequences. The tTF-RGD cDNA insert was confirmed by sequencing.

q. pCR-TK-SE-G6-FLAG: for Insertion of an Expression Cassette Encoding the G6-FLAG Fusion Protein Under the Control of the Vaccinia P_{SE} Promoter into the Vaccinia TK Locus

Vector pCR-TK-SE-G6-FLAG (SEQ ID NO: 100) was employed to develop strain GLV-1h107 having the following genotype: F14.5L: (P_{SEL})Ruc-GFP; TK: (P_{SE}) G6-FLAG; HA: (P₁₁ₖ)gusA. Strain GLV-1h107 was generated by inserting DNA encoding a G6-FLAG fusion protein (SEQ ID NO: 99; G6 is the anti-VEGF scAb) into the TK locus of strain GLV-1h68 thereby deleting the rTrfR-LacZ expression cassette at the TK locus of strain GLV-1h68. Vector pCR-TK-SE-G6-FLAG contains a DNA fragment encoding the G6-FLAG fusion protein operably linked to the vaccinia synthetic early promoter (P_{SE}), sequences of the TK gene flanking the (P_{SE})-fusion protein-encoding DNA fragment, the E. coli guanine phosphoribosyltransferase (gpt) gene under the control of the vaccinia virus P7.5k early and late promoter for transient dominant selection of virus that has incorporated the vector, and sequences of the pUC plasmid.

cDNA encoding G6-FLAG was obtained from vector pGA4-G6 (GeneArt; SEQ ID NO: 98). The vector contains DNA encoding an artificially synthesized single chain antibody (scAb) directed against VEGF (scFv anti-VEGF). The gene encodes the kappa light chain leader sequence for the secretion of the protein, the sequence of the V_{H} domain of the scAb followed by a linker sequence and the sequence of the V_{L} domain of the scAb. The C-terminal end of the gene is fused to DNA encoding a FLAG-tag for ease of protein detection. The 5′ end the G6-FLAG fragment contains a SalI site, and the 3′ end contains a PacI site.

To generate vector pCR-TK-SE-G6-FLAG, the G6-FLAG fragment was released by SalI and PacI restriction enzyme digest of pGA4-G6 (SEQ ID NO: 98) and inserted into TK-SE-CSF-2 (see (n) above; SEQ ID NO: 110), precut with SalI and PacI, to generate plasmid pCR-TK-SE-G6-FLAG (SEQ ID NO: 100), thus placing the G6-FLAG cDNA under the control of vaccinia virus synthetic early (P_{SE}) promoter and in between the left and right TK gene flanking sequences. The G6-FLAG cDNA insert was confirmed by sequencing.

r. pCR-TK-SEL-G6-FLAG: for Insertion of an Expression Cassette Encoding the G6-FLAG Fusion Protein Under the Control of the Vaccinia P_{SEL} Promoter into the Vaccinia TK Locus

Vector pCR-TK-SEL-G6-FLAG (SEQ ID NO: 101) was employed to develop strain GLV-1h108 having the following genotype: F14.5L: (P_{SEL})Ruc-GFP; TK: (P_{SEL})G6-FLAG; HA: (P₁₁ₖ)gusA. Strain GLV-1h108 was generated by inserting DNA encoding a G6-FLAG fusion protein (SEQ ID NO: 99) into the TK locus of strain GLV-1h68 thereby deleting the rTrfR-LacZ expression cassette at the TK locus of strain GLV-1h68. Vector pCR-TK-SEL-G6-FLAG contains a DNA fragment encoding the G6-FLAG fusion protein operably linked to the vaccinia synthetic early/late promoter (P_{SEL}), sequences of the TK gene flanking the (P_{SEL})-fusion protein-encoding DNA fragment, the E. coli guanine phosphoribosyltransferase (gpt) gene under the control of the vaccinia virus P7.5k early and late promoter for transient dominant selection of virus that has incorporated the vector, and sequences of the pUC plasmid.

To generate vector pCR-TK-SEL-G6-FLAG, the G6-FLAG fragment was released by SalI and PacI restriction enzyme digest of pGA4-G6 (see (t) above; SEQ ID NO: 98) and inserted into TK-SEL-CSF-2 (see (o) above; SEQ ID NO: 112), precut with SalI and Pad, to generate plasmid pCR-TK-SEL-G6-FLAG (SEQ ID NO: 101), thus placing the tTF-RGD cDNA under the control of vaccinia virus synthetic early/late (P_{SEL}) promoter and in between the left and right TK gene flanking sequences. The G6-FLAG cDNA insert was confirmed by sequencing.

s. pCR-TK-SL-G6-FLAG: for Insertion of an Expression Cassette Encoding the G6-FLAG Fusion Protein Under the Control of the Vaccinia P_{SL} Promoter into the Vaccinia TK Locus

Vector pCR-TK-SL-G6-FLAG (SEQ ID NO: 102) was employed to develop strain GLV-1h109 having the following genotype: F14.5L: (P_{SEL})Ruc-GFP; TK: (P_{SL}) G6-FLAG; HA: (P₁₁ₖ)gusA. Strain GLV-1h109 was generated by inserting DNA encoding a G6-FLAG fusion protein (SEQ ID NO: 99) into the TK locus of strain GLV-1h68 thereby deleting the rTrfR-LacZ expression cassette at the TK locus of strain GLV-1h68. Vector pCR-TK-SL-G6-FLAG contains a DNA fragment encoding the G6-FLAG fusion protein operably linked to the vaccinia synthetic late promoter (P_{SL}), sequences of the TK gene flanking the (P_{SL})-fusion protein-encoding DNA fragment, the E. coli guanine phosphoribosyltransferase (gpt) gene under the control of the vaccinia virus P7.5k early and late promoter for transient dominant selection of virus that has incorporated the vector, and sequences of the pUC plasmid.

To generate vector pCR-TK-SL-G6-FLAG, the G6-FLAG fragment was released by SalI and Pad restriction enzyme digest of pGA4-G6 (see (t) above; SEQ ID NO: 98) and inserted into TK-SL-CSF-3 (see (p) above; SEQ ID NO: 114), precut with SalI and PacI to generate plasmid pCR-TK-SL-G6-FLAG (SEQ ID NO: 102), thus placing the G6-FLAG cDNA under the control of vaccinia virus synthetic late (P_{SL}) promoter and in between the left and right TK gene flanking sequences. The G6-FLAG cDNA insert was confirmed by sequencing.

t. pF14.5-SEL-RG: for Insertion of an Expression Cassette Encoding the Ruc-GFP Fusion Protein Under the Control of the Vaccinia P_{SEL} Promoter into the Vaccinia F14.5L Locus

pF14.5-SEL-RG (SEQ ID NO: 104) is a targeting vector that can be employed to facilitate insertion of foreign genes in the F14.5L locus of LIVP.

The ruc-gfp fusion cDNA from pcDNA-RG (see, for example, Wang et al., 2002) was amplified by PCR using AccuPrime pfx SuperMix (Invitrogen), using primer that comprise the vaccinia synthetic early/late promoter (P_{SEL}), which places the ruc-gfp under the control of P_{SEL} promoter:

(SEQ ID NO: 117)
5′-ATCAAGCTTAAAAATTGAAATTTTATTTTTTTTTTTTGGAATATA
AATGACTTCGAAAGTTTATGATCCAGAAC-3′
and
(SEQ ID NO: 118)
5′-TCACTTGTACAGCTCGTCCA-3′.

The resulting PCR product was cloned into pCR-Blunt II-TOPO vector (Invitrogen; SEQ ID NO: 40) to yield pCRII-SEL-RG (SEQ ID NO: 105). The vector was sequence confirmed.

To generate vector pF14.5-SEL-RG, the SEL-RG cDNA fragment was released from pCRII-SEL-RG (SEQ ID NO: 105) by Hind III and EcoR V restriction enzyme digest and inserted into pNCVVf14.51T (SEQ ID NO: 11), precut with Hind III and BamH I (blunt ended) to generate plasmid pF14.5-SEL-RG (SEQ ID NO: 104), thus placing the Ruc-GFP fusion cDNA under the control of vaccinia virus synthetic early/late (P_{SEL}) promoter and in between the left and right F14.5L gene flanking sequences.

u. FSE-hNET: for Insertion of an Expression Cassette Encoding hNET Under the Control of the Vaccinia P_{SE} Promoter into the Vaccinia F14.5L Locus

The FSE-hNET vector (SEQ ID NO.: 119) was employed to create vaccinia virus strain GLV-1h99, having the following genotype: F14.5L: (P_{SE})hNET, TK: (P_{SEL})rTrfR-(P_{7.5k})LacZ, HA: (P₁₁ₖ)gusA. FSE-hNET contains the human norepinephrine transporter (hNET) under the control of the vaccinia P_{SE} promoter, flanked by sequences of the F14.5L gene.

To generate the FSE-hNET vector, DNA encoding hNET was PCR amplified from the plasmid pBluescript II KS+-hNET as the template with the following primers:

hNET5 (5′-GTCGACGCCACCATGCTTCTGGCGCGGATGAA-3′, SEQ ID NO: 120) (Sal I restriction site underlined) and hNET3 (5′-GATATCTCAGATGGCCAGCCAGTGTT-3′, SEQ ID NO: 121) (EcoR V site underlined). The PCR product was gel-purified, and cloned into the pCR-Blunt II-TOPO vector (SEQ ID NO: 122) using the Zero Blunt TOPO PCR Cloning Kit (Invitrogen). The resulting construct pCRII-hNET1 confirmed by sequencing. The hNET cDNA was released from pCRII-hNET1 with Sal I and EcoR V enzyme digest, and subcloned into the intermediate vector pCR-SE1 (SEQ ID NO: 123), precut with SalI and SmaI. This step puts hNET cDNA downstream of the sequence for vaccinia virus synthetic early promoter (P_{SE}). The viral hNET expression cassette (SE-hNET) was released from this intermediate construct by BamH I and Hind III enzyme digest, and inserted into the same cut viral transfer vector pNCVVf14.5T (SEQ ID NO: 124). The final construct FSE-hNET1 was confirmed by sequencing and used for insertion of SE-hNET into the F14.5L locus in GLV-1h68.

v. TK-SE-hNET3: for Insertion of an Expression Cassette Encoding hNET Under the Control of the Vaccinia P_{SE} Promoter into the Vaccinia TK Locus

The TK-SE-hNET3 vector (SEQ ID NO.: 125) was employed to create vaccinia virus strain GLV-1h100, having the following genotype: F14.5L: (P_{SEL})Ruc-GFP, TK: (P_{SE})hNET, HA: (P₁₁ₖ)gusA. TK-SE-hNET3 contains the human norepinephrine transporter (hNET) under the control of the vaccinia P_{SE} promoter, flanked by sequences of the TK gene. To generate vector TK-SE-hNET3, hNET cDNA was released from FSE-hNET1 with Sal I and Pac I enzyme digestion, and inserted into same cut vector TK-SE-mIP10 (SEQ ID NO: 126). The resulting construct TK-SE-hNET3 was confirmed by sequencing.

w. TK-SL-hNET3: for Insertion of an Expression Cassette Encoding hNET Under the Control of the Vaccinia P_{SL} Promoter into the Vaccinia TK Locus

The TK-SL-hNET3 vector (SEQ ID NO.: 127) was employed to create vaccinia virus strain GLV-1h101, having the following genotype: F14.5L: (P_{SEL})Ruc-GFP, TK: (P_{SL})hNET, HA: (P₁₁ₖ)gusA. TK-SE-hNET3 contains the human norepinephrine transporter (hNET) under the control of the vaccinia P_{SL} promoter, flanked by sequences of the TK gene. To generate vector TK-SL-hNET3, hNET cDNA was released from FSE-hNET1 with Sal I and Pac I enzyme digestion, and inserted into same cut vector TK-SL-mIP10 (SEQ ID NO: 128). The resulting construct TK-SL-hNET3 was confirmed by sequencing.

x. HA-SE-hNET-1: for Insertion of an Expression Cassette Encoding hNET Under the Control of the Vaccinia P_{SE} Promoter into the Vaccinia HA Locus

The HA-SE-hNET-1 vector (SEQ ID NO: 129) was employed to create vaccinia virus strain GLV-1h139, having the following genotype: F14.5L: (P_{SEL})Ruc-GFP, TK: (P_{SEL})rTrfR-(P_{7.5k})LacZ, HA: (P_{SE})hNET. HA-SE-hNET-1 contains the human norepinephrine transporter (hNET) under the control of the vaccinia P_{SE} promoter, flanked by sequences of the HA gene. To generate vector HA-SE-hNET-1, hNET cDNA was released from TK-SL-hNET-3 (SEQ ID NO.: 127) by Sal I and Pac I enzyme digest, and subcloned into same cut vector HA-SE-RLN-7 (SEQ ID NO.: 130), thereby replacing RLN cDNA with the hNET cDNA. The resulting construct HA-SE-hNET-1 was confirmed by sequencing.

y. HA-SE-IL24-1: for Insertion of an Expression Cassette Encoding IL-24 Under the Control of the Vaccinia P_{SE} Promoter into the Vaccinia HA Locus

The HA-SE-IL24-1 vector (SEQ ID NO.: 131) was employed to create vaccinia virus strains GLV-1h146 and GLV-1h150, having the following genotypes: F14.5L: (P_{SEL})Ruc-GFP, TK: (P_{SE})hNET, HA: (P_{SE})IL-24 and F14.5L: (P_{SEL})Ruc-GFP, TK: (P_{SL})hNET, HA: (P_{SE})IL-24, respectively. HA-SE-IL24-1 contains the human IL-24 gene under the control of the vaccinia P_{SE} promoter, flanked by sequences of the HA gene. To generate vector HA-SE-IL24-1, human IL24 cDNA was PCR amplified using Homo sapiens interleukin 24, transcript variant 1 (cDNA clone MGC:8926) from Origene as the template with the following primers:

mda-5 (5′-GTCGACCACCATGAATTTTCAACAGAGGCTGC-3′, SEQ ID NO.: 132) (Sal I site underlined) and
mda-3 (5′-CCCGGGTTATCAGAGCTTGTAGAATTTCTGCATC-3′, SEQ ID NO.: 133) (Sma I site underlined)). The resulting PCR product was gel purified, and cloned into the pCR-Blunt II-TOPO vector (SEQ ID NO.: 122), using Zero Blunt TOPO PCR Cloning Kit (Invitrogen). The resulting construct pCRII-IL24-3 was sequence confirmed. The IL24 cDNA was released from pCRII-IL24-3 by Sal I and Sma I digest, and subcloned into same cut vector pCR-SE1 (SEQ ID NO.: 123), placing IL24 under the control of vaccinia synthetic promoter (P_{SE}). The resulting construct pCR-SE-IL24-2 was sequence confirmed. The IL24 was then released by Sal I and Pac I enzyme digest, and subcloned into same cut vector HA-SE-RLN-7 (SEQ ID NO.: 130) thereby replacing RLN cDNA with IL-24 cDNA. The resulting constructs HA-SE-IL24-1 was sequence confirmed.

z. HA-SE-hNIS-1: for Insertion of an Expression Cassette Encoding hNIS Under the Control of the Vaccinia P_{SE} Promoter into the Vaccinia HA Locus

The HA-SE-hNIS-1 vector (SEQ ID NO.: 134) was employed to create vaccinia virus strain GLV-1h151, having the following genotype: F14.5L: (P_{SEL})Ruc-GFP, TK: (P_{SEL})rTdR-(P_{7.5k})LacZ, HA: (P_{SE})hNIS. HA-SE-hNIS-1 contains the human sodium iodide symporter (hNIS) under the control of the vaccinia P_{SE} promoter, flanked by sequences of the HA gene. To generate vector HA-SE-hNIS-1, hNIS cDNA was PCR amplified using human cDNA clone TC124097 (SLC5A5) from OriGene as the template with following primers:

hNIS-5
SEQ ID NO.: 135
(5′-GTCGACCACCATGGAGGCCGTGGAGACCGG-3′,)
(Sal I site underlined)
and
hNIS-3
SEQ ID NO.: 135
(5′-TTAATTAATCAGAGGTTTGTCTCCTGCTGGTCTCGA-3′,)
(Pac I site underlined).

The PCR product was gel-purified, and cloned into the pCR-Blunt II-TOPO vector (SEQ ID NO.: 122) using Zero Blunt TOPO PCR Cloning Kit (Invitrogen). The resulting construct pCRII-hNIS-2 confirmed by sequencing. The hNIS cDNA was released from pCRII-hNIS-2 with Sal I and Pac I enzyme digestion, and subcloned into same cut vector HA-SE-RLN-7 (SEQ ID NO.: 130), thereby replacing RLN cDNA. The resulting construct HA-SE-hNIS-1 was confirmed by sequencing.

aa. HA-SEL-hNIS-2: for Insertion of an Expression Cassette Encoding hNIS Under the Control of the Vaccinia P_{SEL} Promoter into the Vaccinia HA Locus

The HA-SEL-hNIS-2 vector (SEQ ID NO.: 137) was employed to create vaccinia virus strain GLV-1h152, having the following genotype: F14.5L: (P_{SEL})Ruc-GFP, TK: (P_{SEL})rTrfR-(P_{7.5k})LacZ, HA: (P_{SEL})hNIS. HA-SEL-hNIS-2 contains the human sodium iodide symporter (hNIS) under the control of the vaccinia P_{SEL} promoter, flanked by sequences of the HA gene. To generate vector HA-SEL-hNIS-2, the hNIS cDNA was released from pCR11-hNIS-2 with Sal I and Pac I enzyme digestion, and subcloned into same cut vector HA-SEL-RLN-2 (SEQ ID NO.: 138), thereby replacing RLN cDNA. The resulting construct HA-SEL-hNIS-2 was confirmed by sequencing.

bb. HA-SL-hNIS-1: for Insertion of an Expression Cassette Encoding hNIS Under the Control of the Vaccinia P_{SL} Promoter into the Vaccinia HA Locus

The HA-SL-hNIS-1 vector (SEQ ID NO.: 139) was employed to create vaccinia virus strain GLV-1h153, having the following genotype: F14.5L: (P_{SEL})Ruc-GFP, TK: (P_{SEL})rTrfR-(P_{7.5k})LacZ, HA: (P_{SL})hNIS. HA-SL-hNIS-1 contains the human sodium iodide symporter (hNIS) under the control of the vaccinia P_{SL} promoter, flanked by sequences of the HA gene. To generate vector HA-SL-hNIS-1, the hNIS cDNA was released from pCRII-hNIS-2 with Sal I and Pac I enzyme digestion, and subcloned into same cut vector HA-SL-RLN-3 (SEQ ID NO.: 140), thereby replacing RLN cDNA. The resulting construct HA-SL-hNIS-1 was confirmed by sequencing.

3. Preparation of Recombinant Vaccinia Viruses

a. GLV-1i69

CV-1 (African green monkey kidney fibroblast) cells (ATCC No. CCL-70), grown in DMEM (Mediatech, Inc., Herndon, Va.) with 10% FBS, were infected with GLV-1h68 at multiplicity of infection (m.o.i.) of 0.1 for 1 hour, then transfected using Lipofectamine 2000 (Invitrogen, Carlsbad, Calif.) with PCR-amplified A34R (SEQ ID NO: 58) coding sequence from VV IHD-J using the following primers: 5′-CATTAATAAATGAAATCGCTTAATAG-3′ (SEQ ID NO: 59) and 5′-GGCGGCGTACGTTAACGAC-3′ (SEQ ID NO: 60). Recombinant virus was selected based on its comet-like plaque morphology as described below.

Two days after transfection, the medium was harvested. To enrich the recombinant extracellular enveloped viruses (EEVs) (i.e. to increase the percentage of recombinant EEV within the infected medium), CV-1 cells were infected with the infected/transfected medium. Two days post infection the infected medium was collected. After the fourth round of the enrichment, the infected medium was diluted and used to infect CV-1 cells. Ten well-isolated plaques were picked and purified for a total of three times. Eight of ten isolates formed comet-like plaques under liquid medium.

b. GLV-1 h and GLV-1j Series

CV-1 cells, grown in DMEM (Mediatech, Inc., Herndon, Va.) with 10% FBS, were infected with the indicated parental viruses (Table 2) at m.o.i. of 0.1 for 1 hr, then transfected using Lipofectamine 2000 or Fugene (Roche, Indianapolis, Ind.) with 2 μg of the corresponding transfer vector (Table 2). Infected/transfected cells were harvested and the recombinant viruses were selected using a transient dominant selection system and plaque purified using methods known in the art (see, e.g., Falkner and Moss, J. Virol., 64, 3108-3111 (1990)). Isolates were plaque purified five times with the first two rounds of plaque isolation conducted in the presence of mycophenolic acid, xanthine and hypoxanthine which permits growth only of recombinant virus that expressing the selectable marker protein, i.e., E. coli guanine phosphoribosyltransferase (gpt), under the control of the vaccinia P_{7.5kE} promoter. As described herein, each of the transfer vectors used in the generation of the GLV-1 h and GLV-1j series of recombinant vaccinia virus contained a (P_{7.5kE})gpt expression cassette. Thus, growth of the virus in the presence of the selection agents enabled identification of virus in which the first crossover event of homologous recombination between the transfer vector and the parental strain genome had occurred. Subsequent growth of the isolates in the absence of selection agents and further plaque purification yielded isolates that had undergone a second crossover event resulting in deletion of the DNA encoding guanine phosphoribosyltransferase from the genome. This was confirmed by the inability of these isolates to grow in the presence of selection agents.

4. Verification of Vaccinia Virus Strain Genotypes

The genotypes of the modified vaccinia virus strains were verified by PCR and restriction enzyme digestion. The nucleotide sequence of the coding sequence from the IHD-J A34R gene (SEQ ID NO: 58) in GLV-1i69 was further verified by sequencing. Lack of expression of the gusA gene in GLV-1h70, GLV-1h73, GLV-1h74, GLV-1h82, GLV-1h83, GLV-1h84, GLV-1h86, GLV-1h90, GLV-1h91 and GLV-1h92 was confirmed by X-GlcA (5-bromo-4-chloro-3-indolyl-β-D-glucuronic acid) staining of the infected cells. Viruses lacking gusA expression are unable to convert the X-GlcA substrate as indicated by lack of development of blue color in the assay as compared to a control strain (e.g. GLV-1h68). Lack of expression of the GFP gene in GLV-1h71, GLV-1h73, GLV-1h74, GLV-1h84, GLV-1h85, GLV-1h96, GLV-1h97 and GLV-1h98 was confirmed by fluorescence microscopy as compared to a control strain (e.g. GLV-1h68). Lack of expression of β-galactosidase in GLV-1h72, GLV-1h74, GLV-1h81, GLV-1h84, GLV-1h85, GLV-1h86, GLV-1h104, GLV-1h105, GLV-1h106, GLV-1h107, GLV-1h108 and GLV-1h109 was confirmed by X-gal (5-bromo-4-chloro-3-indolyl-β-D-galactopyranoside) staining of the infected cells. Viruses lacking lacZ expression are unable to convert the X-gal substrate as indicated by lack of development of blue color in the assay as compared to a control strain (e.g. GLV-1h68). Standard techniques for X-GlcA and X-gal viral staining and fluorescence microscopy were employed and are well-known in the art.

Expression of mRFP in GLV-1h84 was confirmed using a Leica DMI 6000 B fluorescence microscope at 2 days post-infection of CV-1 cells and compared to mock infected cells or non-mRFP expression strains (e.g., GLV-1h73). In one example, the GLV-1h84 infected cells expressed over 2.2×10¹⁰ relative light units compared to no expression in the GLV-1h73 strain. Expression of firefly luciferase in GLV-1h84 was confirmed at two days post-infection of CV-1 cells performed using the Chroma-Glo luciferase assay systems (Promega) and relative light units (RLU) were measured using a Turner TD-20e luminometer.

### B. Vaccinia Virus Purification

Ten T225 flasks of confluent CV-1 cells (seeded at 2×10⁷ cells per flask the day before infection) were infected with each virus at m.o.i. of 0.1. The infected cells were harvested two days post infection and lysed using a glass Dounce homogenizer. The cell lysate was clarified by centrifugation at 1,800 g for 5 min, and then layered on a cushion of 36% sucrose, and centrifuged at 13,000 rpm in a HB-6 rotor, Sorvall RC-5B Refrigerated Superspeed Centrifuge for 2 hours. The virus pellet was resuspended in 1 ml of 1 mM Tris, pH 9.0, loaded on a sterile 24% to 40% continuous sucrose gradient, and centrifuged at 26,000 g for 50 min. The virus band was collected and diluted using 2 volumes of 1 mM Tris, pH 9.0, and then centrifuged at 13,000 rpm in a HB-6 rotor for 60 min. The final virus pellet was resuspended in 1 ml of 1 mM Tris, pH 9.0 and the titer was determined in CV-1 cells (ATCC No. CCL-70).

### Example 2

### Infection of Embryonic Stem Cells with GLV-1h68

Various human and mouse embryonic stem (ES) cell lines and cell line derivatives and were infected with GLV-1h68 to determine whether the virus could infect and replicate in such cells. The virus was used to infect human HUES-1, HUES-7 and H9 stem cells, and embryoid bodies and blood progenitor cells derived from H9 cells, and murine E14 and J1 ES cells.

A. Infection of HUES-1 and HUES-7 Cells

Infection of the human embryonic stem cell lines HUES-1 and HUES-7 (Cowan et al. (2004) N Engl J Med 350(13): 1353-6) was performed on confluent HUES-1 and HUES-7 cells grown in 80% Knockout DMEM (Invitrogen) supplemented with 10% KO-Serum Replacement (INVITROGEN GIBCO CAT#10828-018), 10% Plasmanate (Bayer), 5% fetal calf serum (HYCLONE CAT#SH30070.03), 2 mM Glutamax-I (Invitrogen), 1% non-essential amino acids (Invitrogen), 50 units/mL penicillin and 50 ug/mL streptomycin (Invitrogen), 0.055 mM beta-mercaptoethanol (Gibco), 12 ng/mL recombinant hLIF (Chemicon International), and 5 ng/mL bFGF (Invitrogen) in a 10 cm dish. The cells were infected with 1×10⁵ plaque forming units (PFU) of GLV-1h68 at an estimated multiplicity of infection (MOI) of 0.01. The infected cells and EB were grown at 37° C., 5% CO₂. At 24 and 48 hours post infection, the plaques were imaged using an Olympus 1×71 inverted fluorescence microscope (Olympus Corp., Tokyo, Japan) equipped with a MicroFire True Color Firewire microscope digital charge-coupled device (CCD) camera (Optronics, Goleta, Calif.). Plaques were visualized in HUES-1 and HUES-7 cells at 24 and 48 hours post infection using both phase-contrast imaging and fluorescence imaging (to visualize GLV-1h68-encoded GFP expression), demonstrating that GLV-1h68 infected and replicated in both ES cell lines.

B. Infection of H9 Cells and Derivatives.

GLV-1h68 was used to infect the human embryonic stem cell line H9 (Thomson et al. (1998) Science 282(5391): 1145-7.), embyroid bodies (EB) derived from H9 cells and human blood progenitor cells (BC) derived from the EB. H9 cells and EB derived from the H9 cells 3 days after induction (for EB induction, H9 cells were grown in Stemline II medium supplemented with 50 ng/ml of BMP-4 and VEGF for three days) were infected with GLV-1h68 at a MOI of 0.01. Human blood progenitor cells derived from the EB 3 days after induction (for BC induction EB were grown in Methocult SF H4436 medium supplemented with 50 μg/ml of VEGF, BMP-4, TPO and FLT3-L) were infected with GLV-1h68 at a MOI of 0.1. The infected cells and EB were grown at 37° C., 5% CO₂ before being harvested at 24, 48, and 72 hours post infection and the virus titer determined using a standard plaque assay in CV-1 cells. The plaques were imaged using an Olympus 1×71 inverted fluorescence microscope (Olympus Corp., Tokyo, Japan) equipped with a MicroFire True Color Firewire microscope digital charge-coupled device (CCD) camera (Optronics, Goleta, Calif.).

Plaques were visualized in H9 cells, EB and BC by both phase-contrast imaging and fluorescence imaging (to visualize GLV-1h68-encoded GFP expression), demonstrating that GLV-1h68 infected and replicated in the H9 cells and derivates. Titers of the GLV-1h68 virus also were shown to increase in H9 cells and derivates (Table 3).

TABLE 3
GLV-1h68 titres following infection of H9 cells and derivatives

| | Titre (PFU/10⁶ cells) | | | | | | |
|---|---|---|---|---|---|---|---|
| | | 24 hours post inf. | | 48 hours post inf. | | | |
| | 0 hours | | Std. | | Std. | 72 hours post inf. | |
| | post inf. | Av. | dev. | Av. | dev. | Av. | Std. dev. |
| H9 cells | 1 × 10⁴ | 6.89 × 10³ | 4.28 × 10³ | 4.51 × 10⁵ | 2.99 × 10⁵ | 3.01 × 10⁵ | 5.78 × 10⁴ |
| EB | 1 × 10⁴ | 2.97 × 10³ | 2.15 × 10³ | 7.62 × 10⁴ | 1.24 × 10⁵ | 6.7 × 10⁵ | 1.27 × 10⁵ |
| BC | 1 × 10⁵ | 8.4 × 10⁵ | 7.5 × 10⁵ | 5.3 × 10⁶ | 1.4 × 10⁶ | 1.3 × 10⁷ | 5.6 × 10⁶ |

C. Infection of E14 and J1 Cells.

Mouse embryonic stem cell lines E14 (Wakayama et al., (1999) Proc. Natl. Acad. Sci. 96:14984-14989) and J1 (Hailesellasse Sene et al., (2007) BMC Genomics 8:85), both obtained from University of Wuerzburg, Germany, were maintained in DMEM supplemented with 15% fetal calf serum (FCS) (heat inactivated), 2 mM L-glutamine (Gibco BRL), 1× nonessential amino acids (Gibco BRL), 0.1 mM 2-mercaptoethanol (2-ME), 1000 U/ml recombinant leukemia inhibitory factor (LIF), 50 μg/ml streptomycin, and 50 U/ml penicillin. Routine culture was on a feeder layer of mitomycin-C treated primary embryonic fibroblasts. Cells were cultured in feeder-free conditions for at least 1 week before experiments.

E14 (6.3×10⁶) and J1 (5.1×10⁶) cells were infected with GLV-1h68 virus at MOI of 0.5. After 1 hour infection, the cells were washed twice and replaced with fresh medium. At 0, 20, 30 and 44 hours post infection, the infected ES cultures were imaged using an Olympus 1×71 inverted fluorescence microscope (Olympus Corp., Tokyo, Japan) equipped with a MicroFire True Color Firewire microscope digital charge-coupled device (CCD) camera (Optronics, Goleta, Calif.). Plaques were observed by phase contrast and fluorescence imaging in both cells lines, increasing in number as time progressed, indicating that GLV-1h68 infected and replicated in both E14 and J1 cells.

### Example 3

### Treatment of Teratomas in Rats

The ability of GLV-1h68 to reduce teratoma size is assessed in rats with spinal traumatic injury or transient spinal chord ischemia that have received ES cell transplantation. The ability of GLV-1h68 to reduce teratoma size also is assessed in rats that have received ES cell transplantation but have no spinal injury or ischemia. Clinical signs of teratoma formation typically is seen between 2-3 weeks after cell grafting and is expressed as progressive loss of ambulatory motor function. Histological analysis of spinal cord in such animals shows consistent presence of spinal parenchymal or subdural/epidural teratoma. The effect of administration of GLV-1h68 at various timepoints after ES cell grafting on teratoma size is assessed.

A. Induction of Transient Spinal Chord Ischemia

To induce spinal chord ischemia, adult Sprague Dowley rats are anesthetized using Isoflurane (2-3%) for the entire procedure and body temperature is maintained with a water-heated pad and monitored. A pe-50 catheter is placed in the tail artery for monitoring blood pressure changes. To induce spinal cord ischemia, using aseptic technique, a 1-2 cm cut is made in the lateral thigh and a 2f Fogarty catheter is passed through the left femoral artery to the descending thoracic aorta (approximately 1.5 cm distal to the aortic arch). Baseline data is collected for 15-20 to ensure the animal is stable. The Fogarty balloon catheter is then inflated until arterial pressure drops to approximately 7 mmHg, which is indicative of blood flow occlusion and lower body ischemia. Ischemia is maintained for intervals of 6 to 12 minutes. The balloon is then slowly deflated for reperfusion of spinal cord blood flow, the Fogarty catheter is removed, and the incision is sutured after bupivacaine (0.75%) is infiltrated along the wound edges. During reperfusion (post ischemia), the animal remains lightly anesthetized under Isoflurane anesthesia (approx. 1%), for a period of at least 20 min.

B. Induction of Spinal Traumatic Injury

To induce spinal traumatic injury, adult Sprague Dowley rats, a partial laminectomy of S1-2 vertebra is performed in Isoflurane (2%) anesthetized animals. A Fogarty catheter (2 f) is then placed into Th10 epidural space and inflated with 0.05 cc of saline for 1-5 min. After compression of the spinal cord the catheter is removed and the skin incision is closed with 3-0 silk. As paralysis of the bladder is a common complication after spinal injury and is often complicated with urinary infection, animals receive antibiotics (Gentamicin, 5 mg/kg/day, i.m.) for 7 days after injury, and all animals are observed every 12 hours and Crede's maneuver (manual emptying of the bladder) is performed if necessary.

C. Teratoma Formation

To produce teratomas, ES cells are engrafted into normal rats or rats with spinal traumatic injury or transient spinal chord ischemia. The rats are first anesthetized with Isoflurane (3%) in an anesthetic induction box. Upon loss of responsiveness and spontaneous movement, the rat is removed from the induction box and the back of the animal is shaved between Th10 vertebra and the sacrum. Isoflurane (1-3%) anesthesia is maintained with a mask, which fits onto the muzzle. The surgical field is wiped with alcohol and prepared with Nolvasan solution. A 2-3 cm skin incision is made on midline between L2-L5 vertebra. Paravertebral muscles are removed and the animal is positioned into spinal apparatus (Stoelting spinal unit). To access the spinal cord a partial laminectomy of the L2-L5 vertebral is performed using a dental drill. HUES-7 or HUES-9 ES cells (Cowan et al. (2004) N Engl J Med 350(13): 1353-6) are then delivered into spinal parenchyma using a glass micropipette (100 mm tip diameter). Each animal receives a total of 20 bilateral injections, each injecting 5000 cells in 0.5 μL into central gray matter of L2-L5 segments. After implantation animals are removed from the spinal apparatus, the wound is washed with 2% H₂O₂, infiltrated with 2% Lidocaine solution and the skin is closed in 2 layers using 3-0 silk.

D. Treatment with GLV 1 h-68

Following ES cell transplantation, each rat is administered 5×10⁸ PFU GLV-1h68 in 200 μL PBS at either 2 days, 7 days or 14 days after cell transplantation. A control group of mice receive PBS alone. Animals are perfusion fixed at 1-8 weeks following GLV-1h68- or PBS-treatment, and the GFP expression in the teratomas is verified by fluorescence microscopy. The overall size of the teratomas formed in animals treated with or without GLV-1h68 is assessed and compared.

Since modifications will be apparent to those of skill in this art, it is intended that this invention be limited only by the scope of the appended claims.

1. A method for inhibiting the development of a tumor resulting from cellular therapy in a subject receiving a cellular therapy, comprising:
(a) administering a cellular therapy composition to a subject; and
(b) systemically administering an oncolytic virus to the subject, wherein the oncolytic virus is administered in an amount sufficient to treat or prevent formation of tumors from contaminating tumor cells in the cellular therapy composition.

2. The method of claim 1, where the cellular therapy composition comprises stem cells, bone marrow cells, immune cells, non-immune cells or cells comprising a gene therapy vector.

3-6. (canceled)

7. The method of claim 1, wherein the virus and the cellular therapy composition are administered simultaneously, sequentially or intermittently.

8. The method of claim 1, wherein the cellular therapy composition is pre-treated with the virus prior to administration.

9. The method of claim 1, wherein the cellular therapy composition is administered systemically.

10-11. (canceled)

12. The method of claim 1, wherein the virus is administered in a single administration or multiple administrations.

13. The method of claim 1, wherein the virus is administered after the cellular therapy composition.

14. The method of claim 1, wherein the virus is administered intravenously, or mucosally.

15-23. (canceled)

24. The method of claim 1, wherein the cellular therapy composition treats a disease or disorder selected from among cardiovascular disease, cancer, diabetes, spinal cord injury, neurodegenerative disease, Alzheimer's disease, Parkinson's disease, multiple sclerosis, Amyotrophic lateral sclerosis, Duchenne Muscular Dystrophy, muscle damage or dystrophy, stroke, burns, lung disease, retinal disease, kidney disease, osteoarthritis, and rheumatoid arthritis.

25. The method of claim 1, wherein the cellular therapy composition comprises a cancer stem cells, and the virus infects the cancer stem cells in the cellular therapy composition.

26-35. (canceled)

36. The method of claim 1, wherein the virus is a vaccinia virus.

37. The method of claim 1, wherein the virus is a Lister strain virus.

38. The method of claim 37, wherein the virus is LIVP.

39. The method of claim 37, wherein the virus is selected from among, GLV-1h68, GLV-1i69, GLV-1h70, GLV-1h71, GLV-1h72, GLV-1h73, GLV-1h74, GLV-1h81, GLV-1h82, GLV-1h83, GLV-1h84, GLV-1h85, GLV-1h86, GLV-1j87, GLV-1j88, GLV-1j89, GLV-1h90, GLV-1h91, GLV-1 h92, GLV-1h96, GLV-1h97, GLV-1h98, GLV-1h100, GLV-1h101, GLV-1h139, GLV-1h104, GLV-1h105, GLV-1h106, GLV-1h107, GLV-1h108, GLV-1h109, GLV-1h146, GLV-1h150, GLV-1h151, GLV-1h152 and GLV-1h153.

40. The method of claim 1, wherein the virus is a pox virus.

41. (canceled)

42. The method of claim 1, further comprising detecting the virus in the subject.

43. The method of claim 42, wherein the virus is detected by fluorescence imaging, magnetic resonance imaging (MRI), single-photon emission computed tomography (SPECT), positron emission tomography (PET), scintigraphy, gamma camera, β+ detector, a γ detector or a combination thereof.

44. The method of claim 1, wherein the virus encodes a detectable protein or a protein that induces a detectable signal.

45. The method of claim 44, wherein the detectable protein is selected from among a luciferase or a fluorescent protein.

46. The method of claim 1, wherein the virus encodes a therapeutic gene product.

47. The method of claim 46, wherein the therapeutic gene product is an anti-cancer agent or anti-angiogenic agent.

48. The method of claim 47, wherein the therapeutic gene product is selected from among a cytokine, a chemokine, an immunomodulatory molecule, an antigen, an antibody or fragment thereof, antisense RNA, prodrug converting enzyme, siRNA, angiogenesis inhibitor, a toxin, an antitumor oligopeptide, a mitosis inhibitor protein, an antimitotic oligopeptide, an anti-cancer polypeptide antibiotic, a transporter protein, and tissue factor.

49. The method of claim 1, further comprising administering an anticancer agent.

50. The method of claim 49, wherein the anticancer agent is selected from among a cytokine, a chemokine, a growth factor, a photosensitizing agent, a toxin, an anti-cancer antibiotic, a chemotherapeutic compound, a radionuclide, an angiogenesis inhibitor, a signaling modulator, an anti-metabolite, an anti-cancer vaccine, an anti-cancer oligopeptide, a mitosis inhibitor protein, an antimitotic oligopeptide, an anti-cancer antibody, an anti-cancer antibiotic, an immunotherapeutic agent, hyperthermia or hyperthermia therapy, a bacterium, radiation therapy and a combination of any of the preceding.

51-76. (canceled)
